(19) United States
(12) Patent Application Publication
(10) Pub. No.: US 2026/0216109 A1
(43) Pub. Date: Jul. 30, 2026

(54) TREATMENT OF SKIN DISORDERS WITH SHORT CHAIN FATTY ACIDS

(71) Applicant: Temple University-Of The Commonwealth System of Higher Education, Philadelphia, PA (US)

(72) Inventors: Alla Arzumanyan, Huntington Valley, PA (US); Ira Spector, Jenkintown, PA (US); Mark A. Feitelson, North Wales, PA (US)

(21) Appl. No.: 19/147,069

(22) PCT Filed: Jan. 8, 2024
(86) PCT No.: PCT/US2024/010635

§ 371 Date: Jul. 10, 2025

Related U.S. Application Data
(60) Provisional application No. 63/479,404, filed on Jan. 11, 2023.

(51) Int. Cl. A61K 31/19; A61K 9/00; A61K 31/4035; A61K 31/593; A61K 33/06; A61P 17/06

(52) U.S. Cl. CPC A61K 31/19; A61K 9/0053; A61K 31/4035; A61K 31/593; A61K 33/06; A61P 17/06

(57) ABSTRACT

The present disclosure describes methods of treating a condition comprising administering a pharmaceutical composition comprising at least one short chain fatty acid (SCFA). The disclosed methods can be used to treat a skin disorder or an autoimmune disorder.

### CROSS-REFERENCE

This application claims the benefit of U.S. Provisional Application No. 63/479,404 filed on Jan. 11, 2023, which is incorporated herein by reference in its entirety.

### BACKGROUND

Short chain fatty acids (SCFAs) are saturated aliphatic acids consisting of one polar carboxylic acid moiety and hydrophobic hydrocarbon chain. Among these, acetate (C2), propionate (C3) and butyrate (C4) are the most common and well-studied molecules.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

Disclosed herein is a method for treating or reducing a likelihood of developing a disease or disorder, comprising administering to a subject a therapeutically effective amount of a pharmaceutical composition comprising at least one short chain fatty acid (SCFA), a SCFA precursor, a SCFA biosynthesis precursor, a compound comprising a SFCA moiety, a derivative thereof, and a combination thereof.

Disclosed herein is a method of treating a condition, the method comprising: a) administering a first pharmaceutical composition to a subject in need thereof, wherein the first pharmaceutical composition comprises a therapeutically-effective amount of a compound that is a short chain fatty acid or a pharmaceutically-acceptable salt thereof, and b) administering a second pharmaceutical composition to the subject, wherein the second pharmaceutical composition comprises a therapeutically-effective amount of a phosphodiesterase-4 (PDE4) inhibitor.

Disclosed herein is a method of treating a condition, the method comprising administering to a subject in need thereof a therapeutically-effective amount of a first pharmaceutical composition comprising at least one SCFA and a reduced amount of a second therapy, wherein the reduced amount of the second therapy is therapeutically effective for treating the condition in combination with the therapeutically-effective amount of the first pharmaceutical composition comprising at least one SCFA, and wherein the reduced amount of the second therapy is less than an amount of the second therapy that is therapeutically effective for the condition in absence of the therapeutically-effective amount of the first pharmaceutical composition comprising at least one SCFA.

### BRIEF DESCRIPTION OF THE DRAWINGS

FIG. 1 shows IL-22 levels for the NativeSkin® and InflammaSkin® controls, and following treatment with betamethasone.

FIG. 2 shows Hematoxylin & Eosin (H&E) staining of NativeSkin® samples at Day 0 and Day 7.

FIG. 3 shows H&E staining of InflammaSkin® groups following no treatment or treatment with Betamethasone, Otezla® (Apremilast), or SCFA.

FIG. 4 shows K16 expression of NativeSkin® samples at Day 0 and Day 7.

FIG. 5 shows K16 expression of InflammaSkin® groups following no treatment or treatment with Betamethasone, Otezla® (Apremilast), or SCFA.

FIG. 6 shows IL-17A expression of NativeSkin® samples, and InflammaSkin® groups following no treatment or treatment with Betamethasone, Otezla® (Apremilast), or SCFA comprising butyric acid and propionic acid.

FIG. 7 shows IL-17A expression fold change of InflammaSkin® groups following no treatment or treatment with Betamethasone, Otezla® (Apremilast), or SCFA.

FIG. 8 shows TNF-α expression of NativeSkin® samples, and InflammaSkin® groups following no treatment or treatment with Betamethasone, Otezla® (Apremilast), or SCFA.

FIG. 9 shows TNF-α expression fold change of InflammaSkin® groups following no treatment or treatment with Betamethasone, Otezla® (Apremilast), or SCFA.

FIG. 10 shows cytokine release of IFN-γ, IL-17A, IL-21, IL-22, IL-23, IL-27, Il-31, MIP-3α, and TNF-α from NativeSkin® samples, and InflammaSkin® groups following no treatment or treatment with Betamethasone, Otezla® (Apremilast), or SCFA.

FIG. 11 shows cytokine release of IFN-γ from InflammaSkin® groups following no treatment or treatment with Otezla® (Apremilast) or SCFA.

FIG. 12 shows cytokine release of IL-17A from InflammaSkin® groups following no treatment or treatment with Otezla® (Apremilast) or SCFA.

FIG. 13 shows cytokine release of IL-21 from InflammaSkin® groups following no treatment or treatment with Otezla® (Apremilast) or SCFA.

FIG. 14 shows cytokine release of IL-22 from InflammaSkin® groups following no treatment or treatment with Otezla® (Apremilast) or SCFA.

FIG. 15 shows cytokine release of IL-23 from InflammaSkin® groups following no treatment or treatment with Otezla® (Apremilast) or SCFA.

FIG. 16 shows cytokine release of IL-27 from InflammaSkin® groups following no treatment or treatment with Otezla® (Apremilast) or SCFA.

FIG. 17 shows cytokine release of IL-31 from InflammaSkin® groups following no treatment or treatment with Otezla® (Apremilast) or SCFA.

FIG. 18 shows cytokine release of MIP-3a from InflammaSkin® groups following no treatment or treatment with Otezla® (Apremilast) or SCFA.

FIG. 19 shows cytokine release of TNF-α from InflammaSkin® groups following no treatment or treatment with Otezla® (Apremilast) or SCFA.

FIG. 20 shows ELISA analysis of TNF-α from NativeSkin® samples, and InflammaSkin® groups following no treatment or treatment with Betamethasone, Otezla® (Apremilast), or SCFA.

FIG. 21 shows disease severity score over time in an IMQ psoriasis mouse imiquimod (IMQ) following no treatment or treatment with 25 mg/kg BID Otezla® (Apremilast) or fixed dose BID SCFA.

FIG. 22 shows disease severity score in imiquimod (IMQ)-induced psoriasis model mice following no treatment or treatment with 25 mg/kg BID Otezla® (Apremilast) at day 6 following disease induction on day 0.

FIG. 23 shows disease severity score in imiquimod (IMQ)-induced psoriasis model mice following no treatment or treatment with fixed dose BID SCFA at day 6 following disease induction on day 0.

FIG. 24 shows back skin thickness over time in an imiquimod (IMQ)-induced psoriasis model mice following no treatment or treatment with 25 mg/kg BID Otezla® (Apremilast) or fixed dose BID SCFA.

FIG. 25 shows back skin thickness in imiquimod (IMQ)-induced psoriasis model mice following no treatment or treatment with 25 mg/kg BID Otezla® (Apremilast) at day 6 following disease induction on day 0.

FIG. 26 shows back skin thickness in imiquimod (IMQ)-induced psoriasis model mice following no treatment or treatment with fixed dose BID SCFA at day 6 following disease induction on day 0.

FIG. 27 shows ear skin thickness over time in imiquimod (IMQ)-induced psoriasis model mice following no treatment or treatment with 10 mg/kg etanercept, 25 mg/kg BID Otezla® (Apremilast) or fixed dose BID SCFA.

FIG. 28 shows ear skin thickness in imiquimod (IMQ)-induced psoriasis model mice following no treatment or treatment with 25 mg/kg BID Otezla® (Apremilast) at day 6 following disease induction on day 0.

FIG. 29 shows ear skin thickness in imiquimod (IMQ)-induced psoriasis model mice following no treatment or treatment with fixed dose BID SCFA at day 6 following disease induction on day 0.

FIG. 30 shows ear skin thickness in imiquimod (IMQ)-induced psoriasis model mice following no treatment or treatment with 10 mg/kg etanercept, 25 mg/kg BID Otezla® (Apremilast) or fixed dose BID SCFA at day 6 following disease induction on day 0.

FIG. 31 shows transepidermal water loss (TEWL) over time in imiquimod (IMQ)-induced psoriasis model mice following no treatment or treatment with 25 mg/kg BID Otezla® (Apremilast) or fixed dose BID SCFA.

FIG. 32 shows percentage body weight loss over time in imiquimod (IMQ)-induced psoriasis model mice following no treatment or treatment with 25 mg/kg BID Otezla® (Apremilast) or fixed dose BID SCFA.

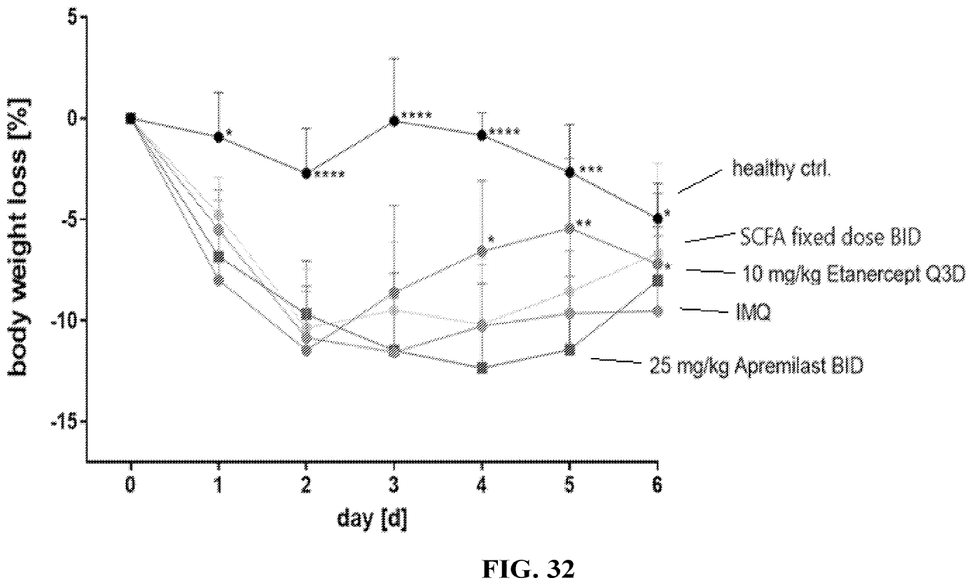

FIG. 33 shows disease severity score over time in imiquimod (IMQ)-induced psoriasis model mice following no treatment or treatment with 7 mg/kg BID Otezla® (Apremilast) or lower dose BID SCFA.

FIG. 34 shows disease severity score in imiquimod (IMQ)-induced psoriasis model mice following no treatment or treatment with 2.5, 5, or 7 mg/kg BID Otezla® (Apremilast), or fixed dose BID SCFA at day 6 following disease induction on day 0.

FIG. 35 shows TNF-α levels in imiquimod (IMQ)-induced psoriasis model mice following no treatment or treatment with 2.5, 5, or 7 mg/kg BID Otezla® (Apremilast), or fixed dose BID SCFA at day 6 following disease induction on day 0.

FIG. 36 shows IL-23 levels in imiquimod (IMQ)-induced psoriasis model mice following no treatment or treatment with 2.5, 5, or 7 mg/kg BID Otezla® (Apremilast), or fixed dose BID SCFA at day 6 following disease induction on day 0.

FIG. 37 shows IL-17A levels in imiquimod (IMQ)-induced psoriasis model mice following no treatment or treatment with 2.5, 5, or 7 mg/kg BID Otezla® (Apremilast), or fixed dose BID SCFA at day 6 following disease induction on day 0.

FIG. 38 shows ear skin thickness over time in imiquimod (IMQ)-induced psoriasis model mice following no treatment or treatment with 2.5, 5, or 7 mg/kg BID Otezla® (Apremilast) alone, fixed dose BID SCFA alone, or with a combination of 2.5, 5, or 7 mg/kg BID Otezla® (Apremilast) and fixed dose BID SCFA.

FIG. 39 shows ear skin thickness over time in imiquimod (IMQ)-induced psoriasis model mice following no treatment or treatment with 2.5 mg/kg BID Otezla® (Apremilast) alone, fixed dose BID SCFA alone, or with a combination of 2.5 mg/kg BID Otezla® (Apremilast) and fixed dose BID SCFA.

FIG. 40 shows ear skin thickness over time in imiquimod (IMQ)-induced psoriasis model mice following no treatment or treatment with 5 mg/kg BID Otezla® (Apremilast) alone, fixed dose BID SCFA alone, or with a combination of 5 mg/kg BID Otezla® (Apremilast) and fixed dose BID SCFA.

FIG. 41 shows ear skin thickness over time in imiquimod (IMQ)-induced psoriasis model mice following no treatment or treatment with 7 mg/kg BID Otezla® (Apremilast) alone, fixed dose BID SCFA alone, or with a combination of 7 mg/kg BID Otezla® (Apremilast) and fixed dose BID SCFA.

FIG. 42 shows percentage body weight loss over time in imiquimod (IMQ)-induced psoriasis model mice following no treatment or treatment with 2.5, 5, or 7 mg/kg BID Otezla® (Apremilast) alone, fixed dose BID SCFA alone, or with a combination of 2.5, 5, or 7 mg/kg BID Otezla® (Apremilast) and fixed dose BID SCFA.

FIG. 43 shows percentage body weight loss over time in imiquimod (IMQ)-induced psoriasis model mice following no treatment or treatment with 2.5 mg/kg BID Otezla® (Apremilast) alone, fixed dose BID SCFA alone, or with a combination of 2.5 mg/kg BID Otezla® (Apremilast) and fixed dose BID SCFA.

FIG. 44 shows percentage body weight loss over time in imiquimod (IMQ)-induced psoriasis model mice following no treatment or treatment with 5 mg/kg BID Otezla® (Apremilast) alone, fixed dose BID SCFA alone, or with a combination of 5 mg/kg BID Otezla® (Apremilast) and fixed dose BID SCFA.

FIG. 45 shows percentage body weight loss over time in imiquimod (IMQ)-induced psoriasis model mice following no treatment or treatment with 7 mg/kg BID Otezla® (Apremilast) alone, fixed dose BID SCFA alone, or with a combination of 7 mg/kg BID Otezla® (Apremilast) and fixed dose BID SCFA.

FIG. 46 shows back skin scaling over time in imiquimod (IMQ)-induced psoriasis model mice following no treatment or treatment with 25 mg/kg BID Otezla® (Apremilast) alone, fixed dose BID SCFA alone, or with a combination of 25 mg/kg BID Otezla® (Apremilast) and fixed dose BID SCFA.

FIG. 47 shows back skin scaling in imiquimod (IMQ)-induced psoriasis model mice following no treatment or treatment with 25 mg/kg BID Otezla® (Apremilast) alone, fixed dose BID SCFA alone, or with a combination of 25 mg/kg BID Otezla® (Apremilast) and fixed dose BID SCFA at day 6 following disease induction on day 0.

FIG. 48 shows cumulative back skin scaling in imiquimod (IMQ)-induced psoriasis model mice following no treatment or treatment with 25 mg/kg BID Otezla® (Apremilast) alone, fixed dose BID SCFA alone, or with a combination of 25 mg/kg BID Otezla® (Apremilast) and fixed dose BID SCFA.

FIG. 49 shows percentage body weight loss over time in imiquimod (IMQ)-induced psoriasis model mice following no treatment or treatment with 25 mg/kg BID Otezla® (Apremilast) alone, fixed dose BID SCFA alone, or with a combination of 25 mg/kg BID Otezla® (Apremilast) and fixed dose BID SCFA.

FIG. 50 shows plasma TNF-α at 6 hours and 26 hours in an LPS animal model treatment with vehicle or fixed dose BID SCFA.

FIG. 51 shows an example of a schematic for assessment of imiquimod (IMQ)-induced psoriasis model mice.

FIG. 52 shows disease severity score over time in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast).

FIG. 53 shows disease severity score in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast) at day 6 following disease induction on day 0.

FIG. 54 shows cumulative disease severity score in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast) following disease induction on day 0 to day 6.

FIG. 55 shows disease severity score in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast) at day 5 following disease induction on day 0.

FIG. 56 shows disease severity score in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast) at day 4 following disease induction on day 0.

FIG. 57 shows back skin scaling over time in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast).

FIG. 58 shows back skin scaling in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast) at day 6 following disease induction on day 0.

FIG. 59 shows back skin scaling in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast) at day 5 following disease induction on day 0.

FIG. 60 shows ear skin thickness over time in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast).

FIG. 61 shows ear skin thickness in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast) at day 6 following disease induction on day 0.

FIG. 62 shows percentage body weight loss over time in imiquimod (IMQ)-induced psoriasis model mice following no treatment or treatment with 10 mg/kg etanercept, 12 mg/kg Otezla® (Apremilast), 25 mg/kg Otezla® (Apremilast), fixed dose BID SCFA, combination of fixed dose BID SCFA and 12 mg/kg Otezla® (Apremilast), or combination of fixed dose BID SCFA and 25 mg/kg Otezla® (Apremilast).

FIG. 63 shows examples of images of healthy control mice.

FIG. 64 shows examples of images of imiquimod (IMQ)-induced psoriasis model control mice.

FIG. 65 shows examples of images of imiquimod (IMQ)-induced psoriasis model mice following treatment with 10 mg/kg etanercept.

FIG. 66 shows examples of images of imiquimod (IMQ)-induced psoriasis model mice following treatment with 12 mg/kg Otezla® (Apremilast).

FIG. 67 shows examples of images of imiquimod (IMQ)-induced psoriasis model mice following treatment with 25 mg/kg Otezla® (Apremilast).

FIG. 68 shows examples of images of imiquimod (IMQ)-induced psoriasis model mice following treatment with fixed dose BID SCFA.

FIG. 69 shows examples of images of imiquimod (IMQ)-induced psoriasis model mice following treatment with combination of fixed dose BID SCFA and 12 mg/kg Otezla® (Apremilast).

FIG. 70 shows examples of images of imiquimod (IMQ)-induced psoriasis model mice following treatment with combination of fixed dose BID SCFA and 25 mg/kg Otezla® (Apremilast).

### DETAILED DESCRIPTION OF THE INVENTION

### Short Chain Fatty Acid (SCFA)

Short chain fatty acids (SCFAs) are fatty acids with aliphatic tails shorter than aliphatic tails of long chain fatty acids. Short chain fatty acids can be derivatized to provide a salt or ester thereof, for example, pharmaceutically-acceptable salts and esters of fatty acids (e.g., sodium butyrate, arginine butyrate).

In some embodiments, a composition disclosed herein comprises at least one SCFA. In some embodiments, a composition disclosed herein comprises at least one short chain fatty acid (SCFA), SCFA precursor, SCFA biosynthesis precursor, a derivative thereof, a SCFA moiety, or a combination thereof.

In some embodiments, a composition disclosed herein comprises at least one SCFA, or a compound comprising a SCFA moiety. In some embodiments, a composition disclosed herein comprises at least two SCFAs. In some embodiments, a composition disclosed herein comprises at least three SCFAs.

Non-limiting examples of a SCFA or SCFA moiety include: acetic acid, butyric acid (BA), C3-C12 fatty acids, C3-C10 fatty acids, C3-C8 fatty acids, methoxyacetic acid, valproic acid (VPA), propionic acid, 3-methoxypropionic acid, ethoxyacetic acid, formic acid, isobutyric acid, tributyrin, N-acetylbutyrate (and other forms of butyrate, e.g., phenylbutyrate, isobutyrate, pivaloyloxymethyl butyrate, monoacetone glucose 3-butyrate), isovaleric acid, valeric acid, isocaproic acid, caproic acid, lactic acid, succinic acid, pyruvic acid, octanoic acid, dodecanoic acid, (4R)-4-hydroxypentanoic acid, 2-ethylhydracrylic acid, 2-hydroxy-3-methylpentanoate, 2-hydroxy-3-methylpentanoic acid, 2-methylbut-2-enoic acid, 2-oxobutanoic acid, 3-hydroxypentanoic acid, 3-methylbut-2-enoic acid, butenoic acid, methylbutyric acid, dimethylbutyric acid, pentadienoic acid, pentenoic acid, pivalic acid, propynoic acid, and a combination thereof.

Non-limiting examples of a SCFA or a SCFA moiety include: compounds or structures with at least 12 carbon atoms, at least 11 carbon atoms, at least 10 carbon atoms, at least 9 carbon atoms, at least 8 carbon atoms, at least 7 carbon atoms, at least 6 carbon atoms, at least 5 carbon atoms, at least 4 carbon atoms, at least 3 carbon atoms, and at least 2 carbon atoms. In some embodiments, the SCFA or SCFA moiety includes compounds or structures with no greater than 13 carbon atoms, no greater than 12 carbon atoms, no greater than 11 carbon atoms, no greater than 10 carbon atoms, no greater than 9 carbon atoms, no greater than 8 carbon atoms, or no greater than 7 carbon atoms.

In some embodiments, a SCFA or SCFA moiety is not a branched fatty acid. In some embodiments, a SCFA or SCFA moiety is a branched fatty acid.

In some embodiments, short chain fatty acids (SCFAs) modulate a cytokine. In some embodiments, the cytokine is a pro-inflammatory cytokine. Non-limiting examples of cytokines include: TNFα, IFNγ, IL-17A, IL-21, IL-22, IL-23, IL-27, IL-31, IL-10, and MIP-3α.

In some embodiments, the cytokine is TNFα. In some embodiments, the cytokine is IL-17. In some embodiments, the cytokine is IL-17A. In some embodiments, the cytokine is IL-22. In some embodiments, the cytokine is IL-23. In some embodiments, the cytokine is IL-10.

In some embodiments, short chain fatty acids (SCFAs) modulate multiple cellular signaling proteins, including, but not limited to, IL-18, TLR3, IFN-γ, TNFα, TGF-β, MyD88, PI3K/Akt, JAK/STAT, Smad 2/3, Smad 4, IL-10, Notch, hedgehog, Wnt (beta-catenin), matrix metalloproteinases 9 and 10, tissue inhibitor of metalloproteinases, nodal and NF-κB signaling. In some embodiments, the signaling proteins that are modulated by SCFAs modulate biological pathways or processes include, but are not limited to, inflammation, immunity, proliferation, differentiation, apoptosis, oncogenesis, transcription of DNA, cytokine production, cell survival, angiogenesis, fibrogenesis and cellular responses to stimuli such as stress, cytokines, free radicals, heavy metals, and ultraviolet irradiation.

In some embodiments, a method disclosed herein comprises treating or reducing a likelihood of developing medical diseases or disorders characterized by elevated levels or abnormal expression of at least one of IL-18, TLR3, IFN-γ, TNFα, TGF-β, MyD88, PI3K/Akt, JAK/STAT, Smad 2/3, Smad 4 or IL-10 signaling. In some embodiments, a method disclosed herein comprises treating or reducing a likelihood of developing medical diseases or disorders characterized by decreased levels or abnormal expression of NF-κB signaling.

In some embodiments, a composition disclosed herein comprises at least one compound comprising a precursor of a SCFA, or a moiety thereof. Non-limiting examples of the precursor include plant cell-wall polysaccharides, dietary nonstarch polysaccharides (NSP) a salt of lactate, a salt of succinate, a salt of formate, 1,2-propenedol, trypamine, indole, indole-3-acetate, and a combination thereof.

In some embodiments, a composition disclosed herein comprises at least one compound comprising a biosynthesis precursor of a SCFA, or a moiety thereof. Non-limiting examples of a biosynthesis precursor include acetyl-CoA carboxylase inhibitor, an adenosine monophosphate kinase (AMPK) activator, vitamin D, and a combination thereof.

In some embodiments, a composition disclosed herein comprises a salt of a SCFA, or a derivative thereof. Non-limiting examples of a salt of butyric acid include sodium butyrate, magnesium butyrate, and calcium butyrate. In some embodiments, the composition comprises one or more of magnesium butyrate and calcium butyrate.

In some embodiments, a composition disclosed herein comprises butyric acid or a pharmaceutically-acceptable salt thereof. In some embodiments, a composition disclosed herein comprises propionic acid or a pharmaceutically-acceptable salt thereof. In some embodiments, a composition disclosed herein comprises butyric acid or a pharmaceutically-acceptable salt thereof and propionic acid or a pharmaceutically-acceptable salt thereof.

In some embodiments, a composition disclosed herein comprises acetic acid or a pharmaceutically-acceptable salt thereof. In some embodiments, a composition disclosed herein comprises butyric acid or a pharmaceutically-acceptable salt thereof, propionic acid or a pharmaceutically-acceptable salt thereof, and acetic acid or a pharmaceutically-acceptable salt thereof.

In some embodiments, a composition disclosed herein comprises a derivative of a SCFA. In some embodiments, the derivative comprises at least one SCFA moiety linked to at least one additional moiety. In some embodiments, the derivative comprises at least one SCFA moiety linked to at least one polyethylene glycol (PEG) moiety. In some embodiments, the SCFA moiety linked to the PEG moiety hydrolyzes under a low pH condition to yield a SCFA molecule and a PEG molecule.

In some embodiments, a composition disclosed herein a combination of SCFAs, and/or derivatives thereof. In some embodiments, the composition is prepared at amounts of at least 10 mM, at least 20 mM, at least 30 mM, at least 40 mM, at least 50 mM, at least 60 mM, at least 70 mM, at least 80 mM, at least 90 mM, at least 100 mM, or more of each or all the compounds of the composition.

Derivatives of SCFAs, e.g., having substituents on the carbon chain such as OH, SH, NH₂, methyl, ethyl, halogen, and other groups that do not interfere with the compound's therapeutic activity can also be used. In some embodiments, a compound disclosed herein comprises at least one SCFA linked to at least one additional moiety, such as OH, SH, NH₂, methyl, ethyl, halogen, and other groups that do not interfere with the compound's therapeutic activity. In some embodiments, a SCFA is pegylated.

In some embodiments, a composition disclosed herein comprises a precursor of a SCFA alone or in combination with one or more SCFAs. Non-limiting examples of precursors of SCFAs include: a salt of formate, a salt of lactate, a salt of succinate, 1,2-propenedol, trypamine, indole, and indole-3-acetate.

In some embodiments, a composition disclosed herein comprises a precursor of SCFA biosynthesis alone or in combination with one or more SCFA. Non-limiting examples of precursors of SCFA biosynthesis include: a salt of formate, a salt of lactate, a salt of succinate, acetyl-CoA carboxylase inhibitors, adenosine monophosphate kinase (AMPK) activators, and vitamin D.

In some embodiments, a compound comprising at least one SCFA, or a compound comprising a SCFA moiety disclosed herein is combined with one or more compounds, such as one or more additional therapeutic agent, for a particular disease or disorder. In some embodiments, the SCFA is in the same composition as one or more additional therapeutic agents. In some embodiments, the composition comprises at least 1, at least 2, at least 3, at least 4, at least 5, at least 6, at least 7, at least 8, at least 9, at least 10, or more than 10 additional therapeutic agents.

### Psoriasis

Psoriasis is a chronic autoimmune disorder affecting the skin. The clinical appearance of psoriasis results from a dysregulated interaction between immune cells (e.g. dendritic cells [DCs] and T cells) and keratinocytes leading to inflammatory processes which drive the disease. The skin inflammation causes a rapid keratinocyte proliferation ending up in scaling of skin's surface, which shows a common phenotype of dry, raised, red skin lesions (plaques) covered with silvery scales.

Nonlimiting examples of psoriasis include: plaque psoriasis, guttate psoriasis, inverse psoriasis, pustular psoriasis, psoriasis vulgaris, seborrheic psoriasis erythrodermic psoriasis, nail psoriasis, and psoriatic arthritis.

In some embodiments, recruitment of innate and adaptive immune cells, which secrete pro-inflammatory cytokines, contribute to the pathogenesis of psoriasis. Cytokines play a critical role in the onset and progression of disease. Non-limiting examples of cytokines that contribute to the pathogenesis of psoriasis include IL-23 and IL-17.

Furthermore, Tregs are impaired in suppressive function leading to an altered T-helper 17/Treg balance. In some embodiments, cytokine antagonists (e.g. anti-TNFs like for instance Etanercept) are used in the treatment of plaque psoriasis (Ps) and psoriatic arthritis (PsA).

The IL-23/IL-17 immune axis can drive skin inflammation in psoriasis, which results from the interplay between keratinocytes and immune cells, such as Th17 cells. In some embodiments, pharmacological blockade of the IL-23/IL-17 immune axis results in clinical efficacy in psoriasis. In some embodiments, pharmacological modulation of the IL-23/IL-17 immune axis is evaluated by different drug modalities and administration (e.g., topical and systemic).

Human Th17 differentiation requires IL-1B, IL-6 and TGF-β and IL-23 to sustain production of IL-17 and IL-22 from Th17 cells. IL-17 induces expression of other pro-inflammatory mediators in keratinocytes, such as IL-17C, IL-19 and IL-36 that, together with IL-17 and IL-22, contribute to keratinocyte activation and epidermal hyperplasia, associated with expression of Keratin-16 and S100A7. In addition, increased levels of IFN-γ in psoriatic skin by activation of skin-resident Th1 cells contribute to the inflammatory activation of keratinocytes.

An autoimmune disease or disorder can include an immune response against a self-antigen that results in inflammation or destruction of healthy tissue in a subject, for example, a mammal, such as a human. Non-limiting examples of autoimmune diseases include arthritis (e.g., rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, psoriasis vulgaris, inverse psoriasis, erythrodermic psoriasis, seborrheic psoriasis and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis, respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions, allergic reaction, eczema including allergic or atopic eczema, asthma such as asthma bronchiale, bronchial asthma, and auto-immune asthma, conditions involving infiltration of T cells and chronic inflammatory responses, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenia purpura (TTP) and autoimmune or immune-mediated thrombocytopenia such as idiopathic thrombocytopenia purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Celiac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine ophthalmopathy, uveoretinitis, chorioretinitis, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia areata, CREST syndrome (calcinosis, Raynaud's phenomenon, esophageal dysmotility, sclerodactyly, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, fibrosis of any organ or tissue, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Leishmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In some embodiments, a method disclosed herein comprises treating a condition. In some embodiments the condition is a skin disorder. In some embodiments, the condition is psoriasis. In some embodiments, treating comprises causing disappearance of a psoriatic lesion in the subject in need thereof.

In some embodiments, a method disclosed herein comprising a SCFA disclosed herein reduces epidermal/dermal separation. In some embodiments, a method disclosed herein comprising a SCFA disclosed herein reduces a likelihood of degradation of the skin after inflammation.

### Psoriasis Models

Preclinical skin models mimic the inflammatory features of psoriasis.

### InflammaSkin® Psoriasis Model

The InflammaSkin® human psoriasis model is an ex vivo psoriasis skin model with a T-cell driven inflammation featuring the Th1/Th17 phenotype. This model allows for evaluation of the response of real human skin. Non-limiting examples of evaluation using an ex vivo psoriasis skin model include: evaluating response to prophylactic biologics, therapeutic biologics, and small molecule drugs after topical or subcutaneous administration.

Mouse imiquimod (IMQ)-induced psoriasis model induced psoriasis is a model representing most features of human psoriasis including phenotypical and histological characteristics of skin lesions and involvement of IL-23/IL-17 axis.

The mouse imiquimod (IMQ) psoriasis model is induced, for example, by the topical administration of Aldara cream (containing 5% of the TLR7/8 ligand IMQ; Meda Pharma GmbH) to the shaved back skin and the ear of female Balb/c (approx. 20 g) mice, is used to evaluate pathogenic mechanisms involved in psoriasis development, and to analyze possible new therapies for psoriasis.

### Mouse Imiquimod (IMQ) Model

Daily repeated applications of IMQ over 6 consecutive days rapidly induces skin inflammation in mice with pathologic and histologic features to human psoriasis, including the development of skin erythema and scaling, epidermal thickening (acanthosis), altered keratinocyte differentiation, neoangiogenesis, and skin infiltration of immune cells. The involvement of a dysregulated IL-23/IL-17 axis and the overproduction of other inflammatory cytokines, like IL-1, IL-36, and IL-22, which are pathways involved in human psoriasis, are mirrored in the IMQ-induced psoriasis.

### Conditions

The present disclosure describes methods and systems for treating conditions comprising administering a pharmaceutical composition comprising at least one short chain fatty acid (SCFA), a SCFA precursor, a SCFA biosynthesis precursor, a compound comprising a SFCA moiety, a derivative thereof, and a combination thereof. In some embodiments, the condition is a skin disorder. In some embodiments, the condition is psoriasis. In some embodiments, the condition is an autoimmune disorder.

Disclosed herein is a method for the treatment or reducing a likelihood of developing at least one disease or disorder in a subject, comprising administering to the subject at least one composition comprising a SCFA or a compound comprising a SCFA moiety, optionally in combination with at least one additional agent or therapy.

In some embodiments, a composition disclosed herein increases the number of disease-free days, reduce the severity of a disease or disorder, reduce the risk of developing a disease or disorder, reduce the risk of recurrence of a disease or disorder, or a combination thereof in the subject. In some embodiments, a composition disclosed herein increases the number of disease-free days by at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60% or more in the subject as compared to a subject who is not receiving treatment. In some embodiments, a composition disclosed herein reduces the severity of a disease or disorder by at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60% or more in the subject as compared to a subject who is not receiving treatment. In some embodiments, a composition disclosed herein reduces the risk of developing a disease or disorder by at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60% or more in the subject as compared to a subject who is not receiving treatment. In some embodiments, a composition disclosed herein reduces the risk of recurrence of a disease or disorder by at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60% or more in the subject as compared to a subject who is not receiving treatment.

Non-limiting examples of diseases and disorders that are treated, reduced in likelihood, or ameliorated include: inflammatory diseases and various cancer diseases. Non-limiting examples of inflammatory diseases and disorders include: asthma, arthritis, allergic rhinitis, psoriasis, atopic dermatitis, inflammatory bowel diseases, Crohn's disease, an allergic or autoimmune disease or disorder associated with C-section delivery of a neonate, uveitis, and vasculitis.

### Skin Disorders

In some embodiments, a SCFA disclosed herein is effective in the treatment of a skin disorder. In some embodiments, a combination of at least one SCFA with at least one other skin disorder treatment can be effective as a therapeutic approach for the treatment of a skin disorder.

Disclosed herein is a method for treatment, inhibition, prevention, or reduction of a skin disease or disorder by administering a composition comprising a SCFA, as disclosed herein, to a subject in need thereof, optionally in combination with at least one additional agent or therapy. Non-limiting examples of skin diseases and disorders include: psoriasis, plaque psoriasis, guttate psoriasis, inverse psoriasis, pustular psoriasis, psoriasis vulgaris, seborrheic psoriasis, erythrodermic psoriasis, nail psoriasis, psoriatic arthritis, systemic lupus erythematosus (SLE) rash, scleroderma (systemic sclerosis), diabetes related skin conditions, rheumatoid arthritis and associated skin rashes (rheumatoid vasculitis), melanoma, vitiligo, eczema (atopic dermatitis), dyshidrotic eczema, rosacea, hives, impetigo, cellulitis, contact dermatitis, canker sores, acne, Lichen planus, actinic keratosis, ichthyosis vulgaris, dermatomyositis, and pemphigoid.

Disclosed herein is a method for treatment, inhibition, prevention, or reduction of a skin disease or disorder, comprising administering a composition comprising a SCFA, as disclosed herein, to a subject in need thereof.

In some embodiments, a subject suffering from a skin disorder is a human. In some embodiments, a subject suffering from a skin disorder is a non-human animal.

In some embodiments, a composition disclosed herein comprises at least one SCFA and at least one second compound for use as a therapeutic for the treatment of skin disorders. In some embodiments, a SCFA comprises one or more of formic acid, acetic acid, propionic acid, isobutyric acid, butyric acid, tributyrin, N-acetylbutyrate (and other forms of butyrate), isovaleric acid, valeric acid, isocaproic acid, caproic acid, lactic acid, succinic acid, pyruvic acid, octanoic acid, and dodecanoic acid. In some embodiments, a second compound comprises one or more of a PDE4 inhibitor, an anti-inflammatory compound, a disease-modifying antirheumatic drug (DMARD), an immunosuppressant, a biologic agent, and a Cox-2 inhibitor.

In some embodiments, a composition for use in methods of treating skin disorders comprises 900 mg butyrate, 100 mg propionate, 10 mg apremilast, 10 mg magnesium, and 50 IU vitamin D3.

In some embodiments, an example of a daily oral dosage for use in methods of treating skin disorders comprises about 3600 mg of butyric acid or a pharmaceutically-acceptable salt thereof such as sodium butyrate, about 400 mg propionic acid or a pharmaceutically-acceptable salt thereof such as sodium propionate, about 40 mg of a source of magnesium, for example, magnesium chloride, and about 200 IU Vitamin D3. In some embodiments, an example of a dosage for use in methods of treating skin disorders comprises about 900 to about 1800 mg of butyric acid or a pharmaceutically-acceptable salt thereof such as sodium butyrate, about 100 to about 200 mg propionic acid or a pharmaceutically-acceptable salt thereof such as sodium propionate, about 10 to about 20 mg of a source of magnesium, for example, magnesium chloride, and about 50 about 100 IU Vitamin D3 administered 1-4 times daily. In some embodiments, a composition is administered 1-4 times daily for at least 1 week, at least 2 weeks, at least 3 weeks or for more than 3 weeks.

In some embodiments, an example dosage for use in methods of treating skin disorders comprises about 1 to about 2 g of butyric acid or a pharmaceutically-acceptable salt thereof, for example, sodium butyrate, about 100 mg of propionic acid or a pharmaceutically-acceptable salt thereof, for example, sodium propionate, about 10 to about 15 mg Otezla, about 10 to about 20 mg of a source of magnesium, for example, magnesium chloride, and about 80 to about 100 IU Vitamin D3.

In some embodiments, a composition comprising at least one SCFA is an enteric coated, extended release, and sustained release capsule.

In some embodiments, a method of treating skin disorders comprises administration of an oral formulation of a SCFA in combination with a topical ointment. An example of a topical ointment for use in treating skin disorder comprises about 40% clobetazol (0.05%) cream, about 20% calcipotriene (vit D, 0.005%) cream, about 20% vit E (0.5%) cream, and about 20% salicylic acid (10%) cream. Another example of a topical ointment for use in treating skin disorder comprises about 40% clobetazol (0.05%) cream, about 20% calcipotriene (vit D, 0.005%) cream, about 20% vit E (0.5%) cream, and about 20% zinc cream. In some embodiments, zinc is used together with salicylic acid in a topical ointment.

### Dosing and Administration

In practicing the methods or use provided herein, therapeutically-effective amounts of the compounds described herein are administered to a subject having a disease or condition to be treated. A therapeutically-effective amount can vary widely depending on the severity of the disease, the age and relative health of the subject, the potency of the compounds used, and other factors. Subjects can be, for example, humans, elderly adults, adults, adolescents, pre-adolescents, children, toddlers, infants, or neonates. A subject can be a patient.

The particular dosage of a compound required to treat the condition can depend on the severity of the condition, the route of administration, and related factors that can be decided by the attending physician.

A therapeutically-effective amount of a compound of the present disclosure can be expressed as mg of the compound per kg of subject body mass.

In some embodiments, a therapeutically-effective amount is 1-1,000 mg/kg, 1-500 mg/kg, 1-250 mg/kg, 1-100 mg/kg, 1-50 mg/kg, 1-25 mg/kg, or 1-10 mg/kg. In some embodiments, a therapeutically-effective amount is about 5 mg/kg, about 10 mg/kg, about 25 mg/kg, about 50 mg/kg, about 75 mg/kg, about 100 mg/kg, about 150 mg/kg, about 200 mg/kg, about 250 mg/kg, about 300 mg/kg, about 400 mg/kg, about 500 mg/kg, about 600 mg/kg, about 700 mg/kg, about 800 mg/kg, about 900 mg/kg, about 1,000 mg/kg, about 5 mg/kg, about 10 mg/kg, about 25 mg/kg, about 50 mg/kg, about 75 mg/kg, about 100 mg/kg, about 150 mg/kg, about 200 mg/kg, about 250 mg/kg, about 300 mg/kg, about 400 mg/kg, about 500 mg/kg, about 600 mg/kg, about 700 mg/kg, about 800 mg/kg, about 900 mg/kg, or about 1,000 mg/kg.

A compound described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 325 mg, from about 325 mg to about 350 mg, from about 350 mg to about 400 mg, from about 400 mg to about 425 mg, from about 425 mg to about 450 mg, from about 450 mg to about 500 mg, from about 500 mg to about 525 mg, from about 525 mg to about 550 mg, from about 550 mg to about 600 mg, from about 600 mg to about 625 mg, from about 625 mg to about 650 mg, from about 650 mg to about 700 mg, from about 700 mg to about 725 mg, from about 725 mg to about 750 mg, from about 750 mg to about 800 mg, from about 800 mg to about 825 mg, from about 825 mg to about 850 mg, from about 850 mg to about 900 mg, from about 900 mg to about 925 mg, from about 925 mg to about 950 mg, from about 950 mg to about 1000 mg, from about 1000 mg to about 1025 mg, from about 1025 mg to about 1050 mg, from about 1050 mg to about 1100 mg, from about 1100 mg to about 1125 mg, from about 1125 mg to about 1150 mg, from about 1150 mg to about 1200 mg, from about 1200 mg to about 1225 mg, from about 1225 mg to about 1250 mg, from about 1250 mg to about 1300 mg, from about 1300 mg to about 1325 mg, from about 1325 mg to about 1350 mg, from about 1350 mg to about 1400 mg, from about 1400 mg to about 1425 mg, from about 1425 mg to about 1450 mg, from about 1450 mg to about 1500 mg, from about 1500 mg to about 1525 mg, from about 1525 mg to about 1550 mg, from about 1550 mg to about 1600 mg, from about 1600 mg to about 1625 mg, from about 1625 mg to about 1650 mg, from about 1650 mg to about 1700 mg, from about 1700 mg to about 1725 mg, from about 1725 mg to about 1750 mg, from about 1750 mg to about 1800 mg, from about 1800 mg to about 1825 mg, from about 1825 mg to about 1850 mg, from about 1850 mg to about 1900 mg, 1900 mg to about 1925 mg, from about 1925 mg to about 1950 mg, or from about 1950 mg to about 2000 mg.

A compound described herein can be present in a composition in an amount of about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg about 1225 mg, about 1250 mg, about 1275 mg, about 1300 mg about 1325 mg, about 1350 mg, about 1375 mg, about 1400 mg about 1425 mg, about 1450 mg, about 1475 mg, about 1500 mg about 1525 mg, about 1550 mg, about 1575 mg, about 1600 mg about 1625 mg, about 1650 mg, about 1675 mg, about 1700 mg about 1725 mg, about 1750 mg, about 1775 mg, about 1800 mg about 1825 mg, about 1850 mg, about 1875 mg, about 1900 mg about 1925 mg, about 1950 mg, about 1975 mg, or about 2000 mg.

In some embodiments, a therapeutically-effective amount of a propionate is about 45 μg/mL. In some embodiments, the propionate is Ca-propionate. In some embodiments, a therapeutically-effective amount of a Ca-propionate is about 45 μg/mL. In some embodiments, a therapeutically-effective amount of a butyrate is about 1 mM. In some embodiments, the butyrate comprises Ca-butyrate and/or Mg-butyrate.

In some embodiments, a therapeutically-effective amount of a propionate is about 60 mg/kg/day. In some embodiments, the propionate is Ca-propionate. In some embodiments, a therapeutically-effective amount of a Ca-propionate is about 60 mg/kg/day.

In some embodiments, a therapeutically-effective amount of a butyrate is about 1800 mg/kg/day. In some embodiments, the butyrate comprises Ca-butyrate and/or Mg-butyrate.

In some embodiments, a therapeutically-effective amount of a butyrate is about 850 mg/kg/day. In some embodiments, the butyrate comprises Ca-butyrate and/or Mg-butyrate.

In some embodiments, a therapeutically-effective amount can be administered 1-35 times per week, 1-14 times per week, or 1-7 times per week. In some embodiments, a therapeutically-effective amount can be administered 1-10 times per day, 1-5 times per day, 1 time, 2 times, or 3 times per day.

In some embodiments, a number of doses for a therapeutically-effective amount can be 1, 2, 3, 4, 5, 6, 7, 8, 9, 10 or more than 10.

In some embodiments, a compound disclosed herein can be administered in therapeutically-effective amounts by various forms and routes including, for example, by intravenous, intravitreal, subcutaneous, intramuscular, oral, rectal, aerosol, parenteral, ophthalmic, pulmonary, transdermal, vaginal, otic, nasal, intraocular, and topical administration. Non-limiting examples of parenteral or systemic administration includes subcutaneous, intravenous, intraperitoneal, and intramuscular injections.

In some embodiments, a pharmaceutical composition disclosed herein is generally formulated for oral or topical (i.e., skin, ocular and mucosal surfaces) administration, with the most suitable route in any given case depending on the nature and severity of the condition being treated and on the nature of the particular active agent which is being used.

### Topical Formulations

In some embodiments, a pharmaceutical composition, composition, or compound disclosed herein, can be administered topically, and thus be formulated in a form suitable for topical administration, i.e. as a pH balanced cream preparation. An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin at a more consistent rate, than a formulation having a lesser concentration, all other things being equal.

Formulations suitable for topical administration include, but are not limited to, liquid or semi liquid preparations such as liniments, lotions, oil in water or water in oil emulsions such as creams, ointments or pastes, and solutions or suspensions. Further, formulations suitable for topical administration can be in the form of cremes and liquids including, for example, syrups, suspensions or emulsions, inhalants, sprays, mousses, oils, gels, and solids. Topically administrable formulations can, for example, comprise from about 1% to about 10% (w/w) active ingredient. Although the concentration of the active ingredient can be as high as the solubility limit of the active ingredient in the solvent. Formulations for topical administration can further comprise one or more of the additional ingredients described herein.

Enhancers of permeation may be used. These materials increase the rate of penetration of drugs across the skin. Non-limiting examples of enhancers include ethanol, glycerol monolaurate, PGML (polyethylene glycol monolaurate), and dimethylsulfoxide. Other enhancers include oleic acid, oleyl alcohol, ethoxydiglycol, laurocapram, alkanecarboxylic acids, dimethylsulfoxide, polar lipids, and N-methyl-2-pyrrolidone.

In some embodiments, the topically active pharmaceutical composition is combined with other ingredients such as adjuvants, anti-oxidants, chelating agents, surfactants, foaming agents, wetting agents, emulsifying agents, viscosifiers, buffering agents, or preservatives. In some embodiments, a permeation or penetration enhancer is included in the composition and is effective in improving the percutaneous penetration of the active ingredient into and through the stratum corneum with respect to a composition lacking the permeation enhancer. Non-limiting examples of permeation enhancers include oleic acid, oleyl alcohol, ethoxydiglycol, laurocapram, alkanecarboxylic acids, dimethylsulfoxide, polar lipids, or N-methyl-2-pyrrolidone. In some embodiments, the composition further comprises a hydrotropic agent, which functions to increase disorder in the structure of the stratum corneum, and thus allows increased transport across the stratum corneum. Non-limiting examples of hydrotropic agents include isopropyl alcohol, propylene glycol, and sodium xylene sulfonate.

The topically active pharmaceutical composition should be applied in an amount effective to affect desired changes. In some embodiments, an active compound is present in an amount of from about 0.0001% to about 15% by weight volume of the composition, from about 0.0005% to about 5% of the composition, or from about 0.001% to about 1% of the composition.

### Oral Formulations

In some embodiments, a pharmaceutical composition, composition, or compound disclosed herein, is administered orally, and thus be formulated in a form suitable for oral administration, i.e. as a solid or a liquid preparation. Suitable solid oral formulations include tablets, capsules, pills, granules, and pellets. Suitable liquid oral formulations include solutions, suspensions, dispersions, emulsions, and oils. If formulated as a capsule, the compositions of the present invention comprise, in addition to the active compound and the inert carrier or diluent, a hard gelating capsule. In some embodiments, a formulation for oral administration is an enteric coated, time release capsule.

Formulations suitable for oral administration can be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations can be prepared by any suitable method of pharmacy, which includes bringing into association the active compound and a suitable carrier (which can contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet can be prepared by compressing or molding a powder or granules containing the active compound, optionally with one or more accessory ingredients. Compressed tablets can be prepared by compressing, in a suitable machine, the compound in a free-flowing form, such as a powder or granules optionally mixed with a binder, lubricant, inert diluent, and/or surface active/dispersing agent(s). Molded tablets can be made by molding, in a suitable machine, the powdered compound moistened with an inert liquid binder.

The delayed release dosage units can be coated with the delayed release polymer coating using conventional techniques, e.g., using a conventional coating pan, an airless spray technique, fluidized bed coating equipment (with or without a Wurster insert).

An example method for preparing extended release tablets is by compressing a drug-containing blend, e.g., blend of granules, prepared using a direct blend, wet-granulation, or dry-granulation process. Extended release tablets can also be molded rather than compressed, starting with a moist material containing a suitable water-soluble lubricant. However, tablets are manufactured using compression rather than molding. A method for forming extended release drug-containing blend is to mix drug particles directly with one or more excipients such as diluents (or fillers), binders, disintegrants, lubricants, glidants, and colorants. As an alternative to direct blending, a drug-containing blend can be prepared by using wet-granulation or dry-granulation processes. Beads containing the active agent can also be prepared by any one of a number of techniques, starting from a fluid dispersion. For example, a method for preparing drug-containing beads involves dispersing or dissolving the active agent in a coating suspension or solution containing pharmaceutical excipients such as polyvinylpyrrolidone, methylcellulose, talc, metallic stearates, silicone dioxide, or plasticizers. The admixture is used to coat a bead core such as a sugar sphere having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

Delayed release formulations are created by coating a solid dosage form with a film of a polymer which is insoluble in the acid environment of the stomach, and soluble in the neutral environment of small intestines. The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Examples of coating materials include bioerodible, gradually hydrolyzable, gradually water-soluble, and/or enzymatically degradable polymers, and can be enteric polymers. Enteric polymers become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Non-limiting examples of coating materials for effecting delayed release include cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, e.g., formed from acrylic acid, methacrylic acid, methyl acrylate, ethyl acrylate, methyl methacrylate and/or ethyl methacrylate, and other methacrylic resins that are commercially available under the tradename Eudragit®, including Eudragit® L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials can be used. Multi-layer coatings using different polymers can be applied.

The coating composition can include conventional additives, such as plasticizers, pigments, colorants, stabilizing agents, glidants, etc. A plasticizer reduces the fragility of the coating, and can be represent about 10 wt. % to 50 wt. % relative to the dry weight of the polymer. Non-limiting examples of plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides. A stabilizing agent is used to stabilize particles in the dispersion. Typical stabilizing agents are nonionic emulsifiers such as sorbitan esters, polysorbates and polyvinylpyrrolidone. Glidants can reduce sticking effects during film formation and drying, and can represent approximately 25 wt. % to 100 wt. % of the polymer weight in the coating solution. One glidant is talc. Other glidants such as magnesium stearate and glycerol monostearates can be used. Pigments such as titanium dioxide can be used. Small quantities of an anti-foaming agent, such as a silicone (e.g., simethicone), can be added to the coating composition.

In addition to a therapeutic or diagnostic agent (or possibly other desired molecules for delivery), the particles can include excipients such as a sugar, such as lactose, a protein, such as albumin, and/or a surfactant.

In some embodiments, a treatment regimen comprises daily oral administration of a short chain fatty acid. In some embodiments, about 600 mg of butyric acid or a pharmaceutically-acceptable salt thereof, such as sodium butyrate, is administered 3 times per day (a total of about 1800 mg/day) for at least one week. In some embodiments, a treatment regimen comprises oral administration of two capsules containing about 600 mg of butyric acid or a pharmaceutically-acceptable salt thereof, such as sodium butyrate 3 times per day (a total of about 3600 mg/day) for at least one week. In some embodiments, a treatment regimen comprises oral administration of two capsules containing about 600 mg of butyric acid or a pharmaceutically-acceptable salt thereof, such as sodium butyrate 3 times per day (a total of about 3600 mg/day) for at least one week followed thereafter by oral administration of capsules containing about 600 mg of butyric acid or a pharmaceutically-acceptable salt thereof, such as sodium butyrate 3 times per day (a total of about 1800 mg/day) for at least one week.

Compounds of the present disclosure, whether administered alone, or in combination with a modulator of oxidative stress can be administered to a subject in need of such administration, for example a human or animal patient.

### Pharmaceutical Compositions

A pharmaceutical composition can be a combination of any compounds described herein with other chemical components, such as pharmaceutically acceptable carriers, stabilizers, binders, diluents, dispersing agents, suspending agents, thickening agents, solubilizing agents, or excipients. Such compositions can be in the form of, for example, granules, powders, tablets, capsules, syrup, suppositories, injections, emulsions, elixirs, suspensions, or solutions. The pharmaceutical composition facilitates administration of the compound to an organism.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

In some embodiments, the pharmaceutical composition provided herein comprises a buffer as an excipient. Non-limiting examples of buffers include potassium phosphate, sodium phosphate, phosphate buffer, citrate buffer, saline sodium citrate buffer (SSC), acetate, saline, physiological saline, phosphate buffer saline (PBS), 4-2-hydroxyethyl-1-piperazineethanesulfonic acid buffer (HEPES), 3-(N-morpholino) propanesulfonic acid buffer (MOPS), and piperazine-N,N′-bis(2-ethanesulfonic acid) buffer (PIPES), citric acid monohydrate, sodium dihydrogen phosphate, potassium dihydrogen phosphate, and any combination thereof.

In some embodiments, the pharmaceutical composition provided herein comprises an alcohol as an excipient. Non-limiting examples of alcohols include ethanol, propylene glycol, glycerol, polyethylene glycol, chlorobutanol, isopropanol, xylitol, sorbitol, maltitol, erythritol, threitol, arabitol, ribitol, mannitol, galactilol, fucitol, lactitol, and any combination thereof.

Pharmaceutical preparations can be formulated with polyethylene glycol (PEG). PEGs with molecular weights ranging from about 300 g/mol to about 10,000,000 g/mol can be used. Non-limiting examples of PEGs include PEG 200, PEG 300, PEG 400, PEG 540, PEG 550, PEG 600, PEG 1000, PEG 1450, PEG 1500, PEG 2000, PEG 3000, PEG 3350, PEG 4000, PEG 4600, PEG 6000, PEG 8000, PEG 10,000, and PEG 20,000.

Further excipients that can be used in a composition described herein include, for example, benzalkonium chloride, benzethonium chloride, benzyl alcohol, butylated hydroxyanisole, butylated hydroxytoluene, chlorobutanol, dehydroacetic acid, ethylenediamine, ethyl vanillin, glycerin, hypophosphorous acid, phenol, phenylethyl alcohol, phenylmercuric nitrate, potassium benzoate, potassium metabisulfite, potassium sorbate, sodium bisulfite, sodium metabisulfite, sorbic acid, thimerasol, acetic acid, aluminum monostearate, boric acid, calcium hydroxide, calcium stearate, calcium sulfate, calcium tetrachloride, cellulose acetate pthalate, microcrystalline celluose, chloroform, citric acid, edetic acid, and ethylcellulose.

In some embodiments, the pharmaceutical composition provided herein comprises an aprotic solvent as an excipient. Non-limiting examples of aprotic solvents include perfluorohexane, α,α,α-trifluorotoluene, pentane, hexane, cyclohexane, methylcyclohexane, decalin, dioxane, carbon tetrachloride, freon-11, benzene, toluene, carbon disulfide, diisopropyl ether, diethyl ether, t-butyl methyl ether, ethyl acetate, 1,2-dimethoxyethane, 2-methoxyethyl ether, tetrahydrofuran, methylene chloride, pyridine, 2-butanone, acetone, N-methylpyrrolidinone, nitromethane, dimethylformamide, acetonitrile, sulfolane, dimethyl sulfoxide, and propylene carbonate.

The amount of the excipient in a pharmaceutical composition described herein can be about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 15%, about 20%, about 25%, about 30%, about 35%, about 40%, about 45%, about 50%, about 60%, about 70%, about 80%, about 90%, about 100%, about 200%, about 300%, about 400%, about 500%, about 600%, about 700%, about 800%, about 900%, or about 1000% by mass of a compound in the pharmaceutical formulation.

The amount of the excipient in a pharmaceutical composition described herein can be about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 15%, about 20%, about 25%, about 30%, about 35%, about 40%, about 45%, about 50%, about 55% about 60%, about 65%, about 70%, about 75%, about 80%, about 85%, about 90%, about 95%, about 99%, or about 100% by mass or by volume of the unit dosage form.

In some embodiments, the addition of an excipient to a pharmaceutical composition described herein can increase or decrease the viscosity of the composition by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, the addition of an excipient to a pharmaceutical composition described herein can increase or decrease the viscosity of the composition by no greater than 5%, no greater than 10%, no greater than 15%, no greater than 20%, no greater than 25%, no greater than 30%, no greater than 35%, no greater than 40%, no greater than 45%, no greater than 50%, no greater than 55%, no greater than 60%, no greater than 65%, no greater than 70%, no greater than 75%, no greater than 80%, no greater than 85%, no greater than 90%, no greater than 95%, or no greater than 99%.

A composition disclosed herein may be used as a complete food product, as a component of a food product, as a dietary supplement or as part of a dietary supplement, as a feed additive and may be either in liquid, semisolid or solid form. A composition disclosed herein additionally may be in the form of a pharmaceutical composition. The compositions, dietary supplements, food products, baby food products, feed additives, and/or pharmaceutical compositions disclosed herein may advantageously be utilized in methods for promoting the health of an individual.

A composition disclosed herein may be in liquid, semisolid or solid form. For example, the compositions can be administered as tablets, gel packs, capsules, gelatin capsules, flavored drinks, as a powder that can be reconstituted into such a drink, cooking oil, salad oil or dressing, sauce, syrup, mayonnaise, or margarine. Furthermore, the food product, dietary supplements, can include, but are not limited to, dairy products, baby food, baby formula, beverages, bars, a powder, a food topping, a drink, a cereal, an ice cream, a candy, a snack mix, a baked food product, and a fried food product. Non-limiting examples of beverages include energy drinks, nutraceutical drinks, smoothies, sports drinks, orange juice, and other fruit drinks. Non-limiting examples of a bar include a meal replacement, a nutritional bar, a snack bar and an energy bar, and an extruded bar. Non-limiting examples of a dairy product include yogurt, yogurt drinks, cheese, and milk.

In some embodiments, a food products or dietary supplements can further comprise herbals, herbal extracts, fungal extracts, enzymes, fiber sources, minerals, and vitamins. In some embodiments, a microalgal oils and microalgal biomass can be used in the compositions of the invention for both therapeutic and non-therapeutic uses. Thus, the compositions, food products, and animal feed additives disclosed herein can be used for therapeutic or non-therapeutic purposes.

In some embodiments, a method disclosed herein comprises treating a condition. In some embodiments the condition is a skin disorder. In some embodiments, the condition is psoriasis. In some embodiments, treating comprises causing disappearance of a psoriatic lesion in the subject in need thereof.

In some embodiments, a method disclosed herein comprising an SCFA disclosed herein reduces epidermal/dermal separation. In some embodiments, a method disclosed herein comprising an SCFA disclosed herein prevents degradation of the skin after inflammation.

In some embodiments, a therapeutically-effective amount can be an amount effective in treating a condition, treating a skin disorder, treating psoriasis, or treating an autoimmune disorder.

In some embodiments, a therapeutically-effective amount can be an amount effective in causing disappearance of a psoriatic lesion in the subject in need thereof. The disappearance of a psoriatic lesion can be by at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 99%.

In some embodiments, a therapeutically-effective amount can be an amount effective in reduces epidermal/dermal separation in the skin. The epidermal/dermal separation can be reduced in size by at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 99%.

In some embodiments, a therapeutically-effective amount can be an amount effective in prevents degradation of the skin after inflammation.

### Pharmaceutically-Acceptable Salts.

The present disclosure provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt.

Metal salts can arise from the addition of an inorganic base to a compound described herein. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound described herein. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, piprazole, imidazole, or pyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a piprazole salt, an imidazole salt, or a pyrazine salt.

Acid addition salts can arise from the addition of an acid to a compound described herein. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

The method disclosed herein can involve administering to a patient or subject compounds of the present disclosure alone, or in combination with a second compound.

### Combinations

In some embodiments, a method disclosed herein further comprises administering a therapeutically-effective amount of a second therapy. In some embodiments, the second therapy exhibits synergy with the compound comprising a short chain fatty acid or a pharmaceutically-acceptable salt thereof. In some embodiments, the second therapy exhibits an additive therapeutic effect to the compound comprising a short chain fatty acid or a pharmaceutically-acceptable salt thereof. In some embodiments, the compound comprising a short chain fatty acid or a pharmaceutically-acceptable salt thereof exhibits an additive therapeutic effect to the second therapy.

In some embodiments, a method disclosed herein comprises administering to a subject in need thereof a therapeutically-effective amount of a compound comprising a short chain fatty acid or a pharmaceutically-acceptable salt thereof and a reduced amount of a second therapy, wherein the reduced amount of the second therapy is therapeutically effective for treating the condition in combination with the therapeutically-effective amount of the compound comprising a short chain fatty acid or a pharmaceutically-acceptable salt thereof, and wherein the reduced amount of the second therapy is less than an amount of the second therapy that is therapeutically effective for the condition in absence of the therapeutically-effective amount of the compound comprising a short chain fatty acid or a pharmaceutically-acceptable salt thereof.

In some embodiments, a combination can be in a single formulation or can be separate and administered in sequence (either a composition comprising at least one SCFA, or a molecule comprising a SCFA moiety, first and then a composition comprising an additional agent, or a composition comprising an additional agent first and then a composition comprising at least one SCFA, or a molecule comprising a SCFA moiety). In some embodiments, the at least one SCFA, or a molecule comprising a SCFA moiety, can be administered to the subject about 30 seconds, 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 0.25 hours, 0.5 hours, 0.75 hours, 1 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, 96 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 1 week, 2 weeks, 3 weeks, 4 weeks, 5 weeks, 6 weeks, 7 weeks, or 8 weeks before the composition comprising at least one additional agent is administered to the subject. In other embodiments, the composition comprising at least one additional agent can be administered to the subject about 30 seconds, 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 60 minutes, 0.25 hours, 0.5 hours, 0.75 hours, 1 hours, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, 96 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 1 week, 2 weeks, 3 weeks, 4 weeks, 5 weeks, 6 weeks, 7 weeks, or 8 weeks before the composition comprising at least one SCFA, or a molecule comprising a SCFA moiety, is administered to the subject.

### PDE4 Inhibitors

In some embodiments, a method disclosed herein comprises administering to a subject in need thereof a composition comprising at least one SCFA, or a biologically-active derivative or precursor thereof, in combination with one or more phosphodiesterase 4 (PDE4) inhibitors. Non-limiting examples of PDE4 inhibitors include Apremilast (Otezla®), roflumilast (Daxas®), Crisaborole (5-(4-Cyanophenoxy)-2,3-dihydro-1-hydroxy-2,1-benzoxaborole, AN-2728, Eucrisa®), Pefcalcitol (M5181), and HFP034 (butyl 2-[2-(2-fluorophenyl) acetamido] benzoate). In some embodiments, a method disclosed herein comprises administering to a subject in need thereof a composition comprising at least one SCFA, or a biologically-active derivative or precursor thereof, and additionally one or more PDE4 inhibitor. In some embodiments, a method disclosed herein comprises administering to a subject in need thereof a composition comprising at least one SCFA, or a biologically-active derivative or precursor thereof, in combination with a composition comprising one or more PDE4 inhibitor.

### Biologic Agents

In some embodiments, a method disclosed herein comprises administering to a subject in need thereof a composition comprising at least one SCFA, or a biologically-active derivative or precursor thereof, in combination with one or more biologic drugs. Nonlimiting biologic drugs include: etanercept (Enbrel®), infliximab (Remicade®), Apremilast (Otezla®), and adalimumab (Humira®).

Additional nonlimiting biologic drugs include: ustekinumab, secukinumab, ixekizumab, guselkumab, and risankizumab.

In some embodiments, a method disclosed herein comprises administering to a subject in need thereof a composition comprising at least one SCFA, or a biologically-active derivative or precursor thereof, and additionally one or more biologic agent. In some embodiments, a method disclosed herein comprises administering to a subject in need thereof a composition comprising at least one SCFA, or a biologically-active derivative or precursor thereof, in combination with a composition comprising one or more biologic agent.

### Magnesium, Vitamin D3, and Vitamin E

In some embodiments, a method disclosed herein comprises administering to a subject in need thereof a composition comprising at least one SCFA, or a biologically-active derivative or precursor thereof, and additionally one or more of magnesium, vitamin D3, and vitamin E (d-α-tocopherol acetate). Magnesium is a co-factor for more than 300 enzymes that regulate diverse biochemical reactions including regulation of blood glucose levels, detoxification, and others. Vitamin D3 deficiency is frequent in patients with immune disorders. Vitamin E has distinctive antioxidant activities.

In some embodiments, a method disclosed herein comprises administering to a subject in need thereof a composition comprising at least one SCFA, or a biologically-active derivative or precursor thereof, and additionally one or more of a source of magnesium, vitamin D3, and vitamin E. In some embodiments, a method disclosed herein comprises administering to a subject in need thereof a composition comprising at least one SCFA, or a biologically-active derivative or precursor thereof, in combination with a composition comprising one or more of a source of magnesium, vitamin D3, and vitamin E.

In some embodiments, a composition disclosed herein comprises a source of magnesium. In some embodiments, a composition disclosed herein comprises an inorganic magnesium salt such as magnesium chloride, magnesium carbonate, or magnesium phosphate. In some embodiments, a composition disclosed herein comprises vitamin D3. In some embodiments, a composition disclosed herein comprises vitamin E.

Any embodiments disclosed herein can be used in conjunction or individually. For example, any pharmaceutically-acceptable excipient, method, technique, solvent, or compound disclosed herein can be used together with any other pharmaceutically-acceptable excipient, method, technique, solvent, or compound disclosed herein to achieve any therapeutic result. Compounds, excipients, and other formulation components can be present at any amount, ratio, or percentage disclosed herein in any such formulation, and any such combination can be used therapeutically for any purpose described herein.

### EXAMPLES

### Example 1: Treatment of Skin Disorders with Short Chain Fatty Acids-Inhibition of Th1/Th17 Inflammation

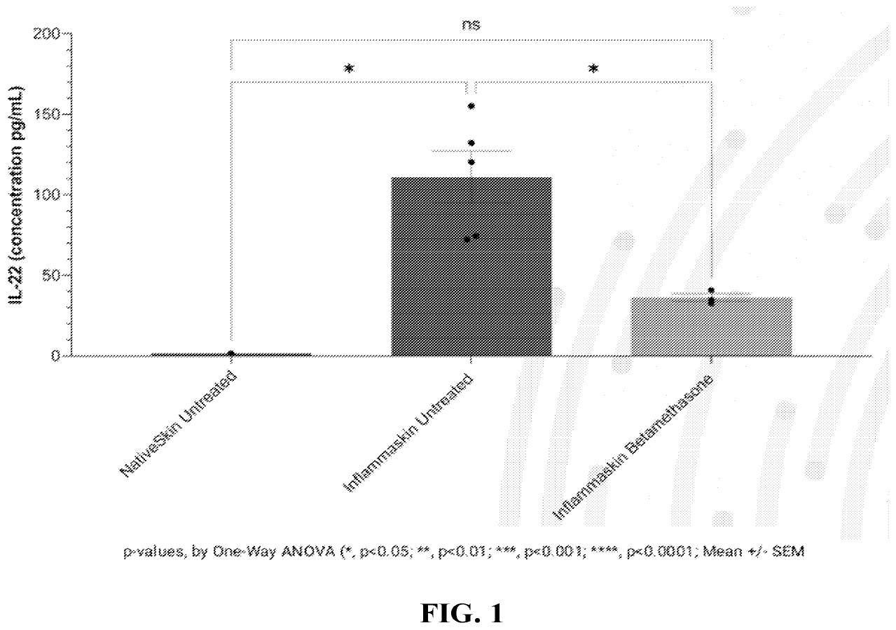

Various assays were designed and tested in an ex vivo human skin psoriasis model (e.g., Genoskin's InflammaSkin® model) as shown and described for FIGS. 1-9. Assays included evaluations of IL-22 cytokine secretion, Hematoxylin & Eosin (H&E) staining (e.g., incidence of pyknosis, vacuolization, and epidermal/dermal detachment), K16 expression in the epidermis, and cytokine expression/secretion of TNF-α and IL-17A.

### PSO-InflammaSkin® Models Culture and Treatment

A total of 16 biopsies of 15 mm diameter were produced from a single donor. Two NativeSkin® models with 12/15 mm silicon rings were produced from the biopsies according to standard procedures in place, and cultured under cell culture conditions (37° C., 5% CO2, max. humidity) for 7 days with 2 mL of standard NativeSkin® medium renewed every day. Fourteen PSO-InflammaSkin® models with 12/15 mm silicon rings were produced by inducing in situ activation and Th17/Th1 polarization of skin resident T cells with a proprietary cocktail, and cultured under cell culture conditions (37° C., 5% CO2, max. humidity) for 7 days with 2 mL of PSO-InflammaSkin® culture medium renewed every day.

From Day 1 to Day 6, models were systemically treated with 1 μM Otezla or Short Chain Fatty Acids (SCFAs), or topically treated with positive control 0.05% Betamethasone

In these experiments, the SCFAs administered were 45 μg/mL of Ca-propionate and 1 mM mixture of salts (Ca-butyrate and Mg-butyrate).

At Day 7, supernatants were collected and stored at −80° C. and skin models of all conditions were carefully unmolded. Skin biopsies were re-punched to eliminate the area under the silicon ring, fixed in 10% buffered formalin and processed for paraffin wax embedding.

### IL-22 Release/ELISA Assay

Immunoassays are used, for example, enzyme linked immunosorbant assays (ELISA). IL-22 release in the supernatant was assessed using the ELISA Human IL-22 kit (Abcam, ref: ab216170). Sample and the standards were analyzed in duplicate. The analysis was carried out on control conditions only to confirm the donor sample's response to T-cell activation and to the positive control, betamethasone. Plates were read on VICTOR Nivo Multimode Microplate Reader. Values are expressed in pg/mL. For each condition, single values, mean and SEM were plotted using GraphPad Prism. Statistical analysis was performed using a One Way ANOVA test.

On Day 7, donor sample's IL-22 levels were >25 μg/mL (~100 μg/mL), indicating that the donor sample was responsive to inflammation induction/T-cell activation. In addition, the donor was also responsive to a reduction in inflammation. FIG. 1 shows a decrease in IL-22 levels, compared to the InflammaSkin® controls, on Day 7 with positive treatment control, betamethasone. Therefore, the results from the donor sample could be used for further analysis.

### Histological Analysis—Hematoxylin & Eosin (H&E) Staining

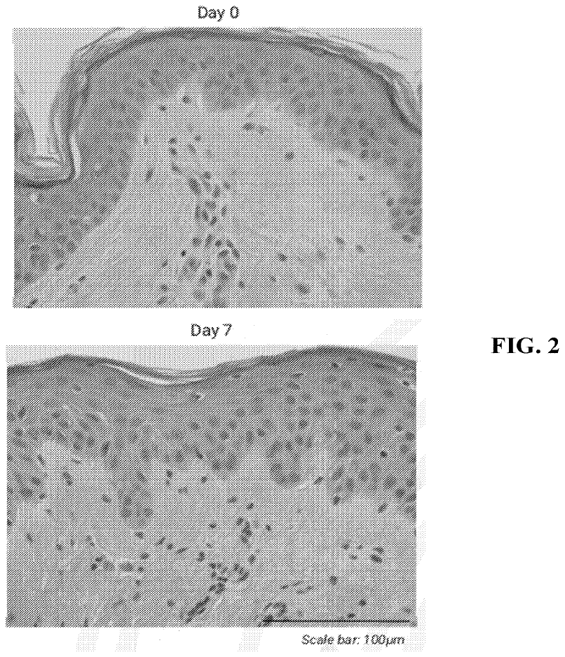

Skin structure and integrity and viability were evaluated using Hematoxylin & Eosin (H&E) staining following treatment with Betamethasone, 10-15 mg Otezla® (Apremilast), or SCFAs comprising butyric acid and propionic acid. H&E analysis features noted included pyknosis, vacuolization, and epidermal/dermal separation. H&E staining was performed on 5 μm thickness paraffin-embedded skin cross-sections. Three representative pictures were acquired at 40× magnification using a Leica DMil microscope. FIG. 2 shows representative images for Day 0 and Day 7 NativeSkin® samples to analyze skin structure integrity and viability. The Day 0 and Day 7 NativeSkin® samples appeared to be healthy with no signs of pyknosis/vacuolization (indicators of cell death) or epidermal/dermal separation. Therefore, the donor sample was determined to remain viable over the course of the culture in the absence of inflammation/T-cell activation.

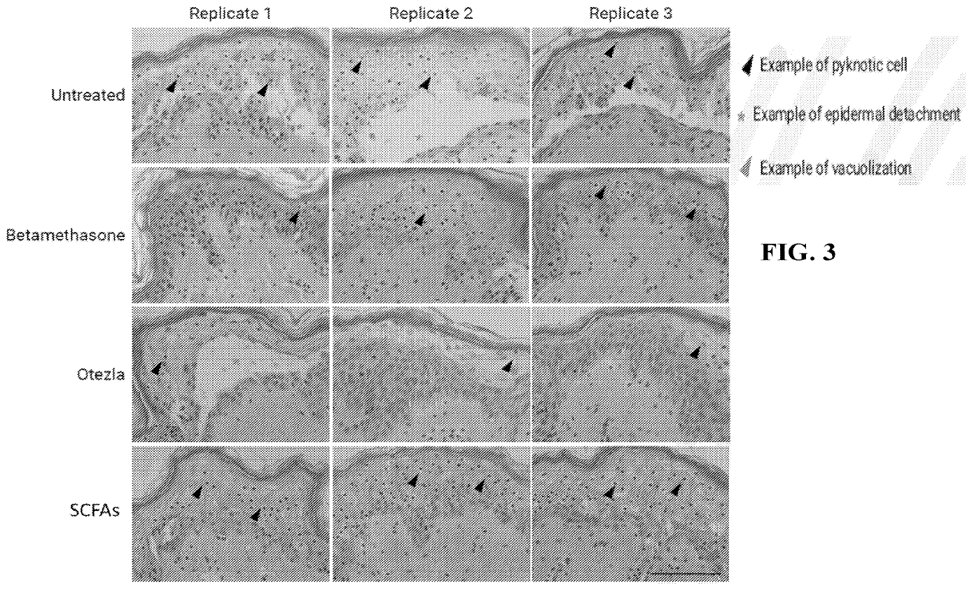

FIG. 3 shows representative images of H&E staining following treatment with Betamethasone, 10-15 mg Otezla® (Apremilast), or SCFAs comprising butyric acid and propionic acid. All 5 replicates of the untreated InflammaSkin® groups showed reduced epidermal cellular viability, as indicated by pyknotic and vacuolized cells, and epidermal/dermal separation.

Betamethasone treatment of the InflammaSkin® models appeared to reduce the incidence of vacuolization and epidermal/dermal separation. Evidence of a large amount of pyknosis, especially in the upper layers of the epidermis, was observed. The largest difference between the betamethasone and the untreated InflammaSkin® models appeared to be the reduction in epidermal/dermal separation. Some variability in the extent of inflammation/damage amongst the three replicates was observed. For example, Replicate 2 appeared to have more similar histology to the untreated InflammaSkin® than to the other 2 replicates.

Otezla systemic treatment of the InflammaSkin® models also appeared to reduce the incidence of pyknosis, vacuolization and epidermal/dermal separation. Variability was observed in the extent of inflammation/damage among the three replicates. Replicate 1 appeared to have retained the most damage to the skin—with epidermal/dermal separation and a large number of pyknotic/vacuolized epidermal cells—appearing to be similar to the untreated InflammaSkin® histological findings. Replicates 2 and 3 displayed some vacuolization and pyknosis in the upper layers of the epidermis, but the basal layers of the epidermis appeared to be healthier. This observation indicates healing of the skin. A larger zone of healthier basal layers of the epidermis was observed than that of the betamethasone treated InflammaSkin® samples was.

Systemic SCFA treatment of the InflammaSkin® models also appeared to increase the health of the skin. Pyknosis/vacuolization of the epidermal layers was observed and closely resembled that of the untreated InflammaSkin® histological findings, much like that of the betamethasone treated InflammaSkin® models. Significantly less epidermal/dermal separation in the SCFA was observed in the treated vs untreated InflammaSkin® models, and suggests that the SCFA has some effect on preventing the degradation of the skin after inflammation induction.

### Histological Analysis-Anti-K16 Immunostaining

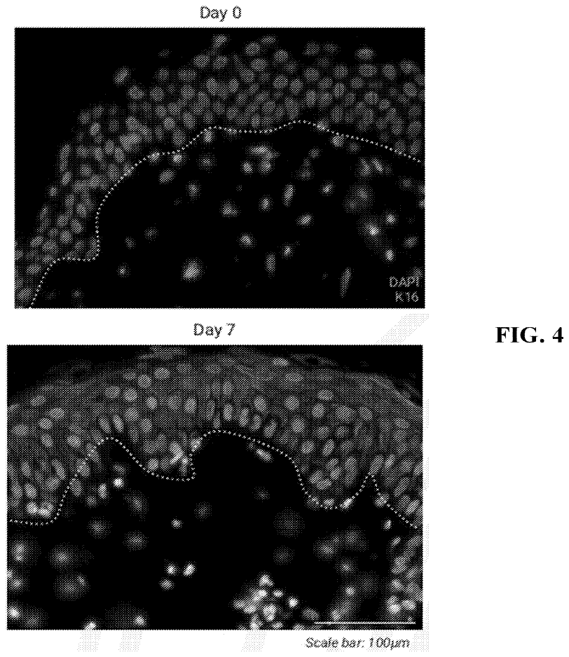

K16, a type 1 keratin, is upregulated in hyperproliferative states, such as in psoriasis. Anti-K16 immunostaining was performed on 5 μm thickness paraffin-embedded skin cross-sections with a primary antibody anti-K16 (Sigma, SAB4501660, Rabbit IgG at 1/100 vol/vol dilution) and a secondary antibody (LifeTech, A21428 Alexa Fluor 647 at 1/500 vol/vol dilution). Representative pictures were acquired at 40× magnification using a Zeiss AxioImager M2 microscope. FIG. 4 shows representative images for anti-K16 immunostaining Day 0 and Day 7 InflammaSkin® models. The Day 7 InflammaSkin® models show K16 was upregulated.

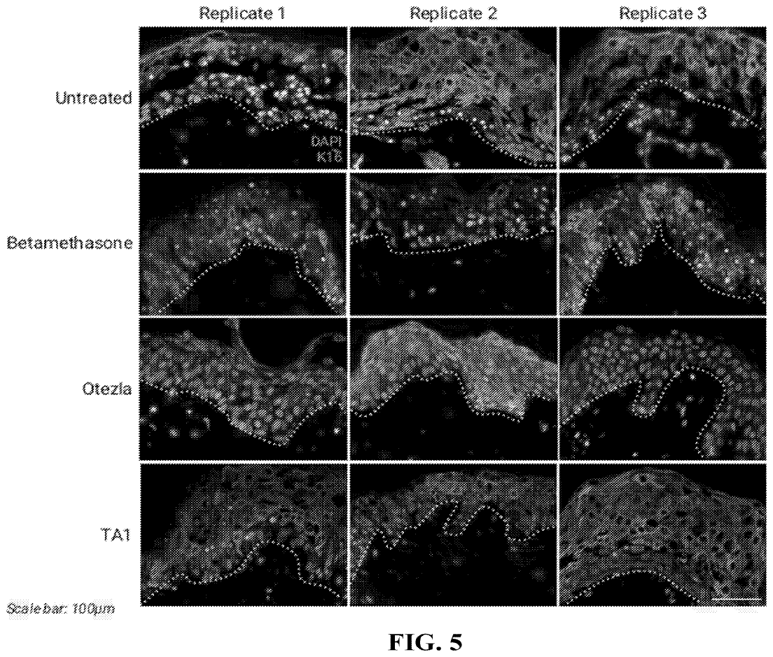

FIG. 5 shows representative images for anti-K16 immunostaining following treatment with Betamethasone, 10-15 mg Otezla® (Apremilast), or SCFAs comprising butyric acid and propionic acid. In Genoskin's InflammaSkin® models, K16 was upregulated in treated samples compared to the uninflamed, NativeSkin® controls where no to very little K16 was expressed. Some variability in the K16 expression was observed across the five replicates. K16 expression (besides Replicate 1) appeared to be expressed almost uniformly from the basal to the corneum epidermal layers.

Betamethasone, Otezla, and SCFA all also decreased the expression of K16 in the epidermis. For Otezla and for SCFAs, less expression of K16 in the basal/suprabasal epidermal layers was observed than in the upper layers of the epidermis (verses expressed throughout the epidermis).

### Cytokine Analysis—Multiplex Assay

Human pro-inflammatory cytokines released in the supernatant were quantified using the V-PLEX Human IL-17A Kit (MesoScale Discovery, reference: K151RFD-1) and V-PLEX Human TNFα Kit (MesoScale Discovery, reference: K151QWD-1). Values are expressed in pg/mL. For each condition, single values, mean and SEM were plotted using GraphPad Prism. Statistical analysis was performed using a One Way ANOVA test.

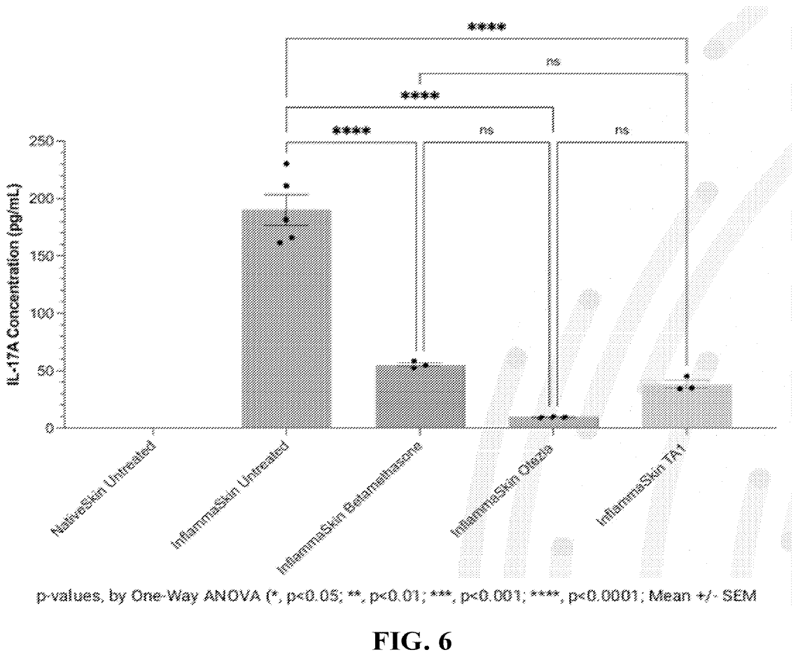

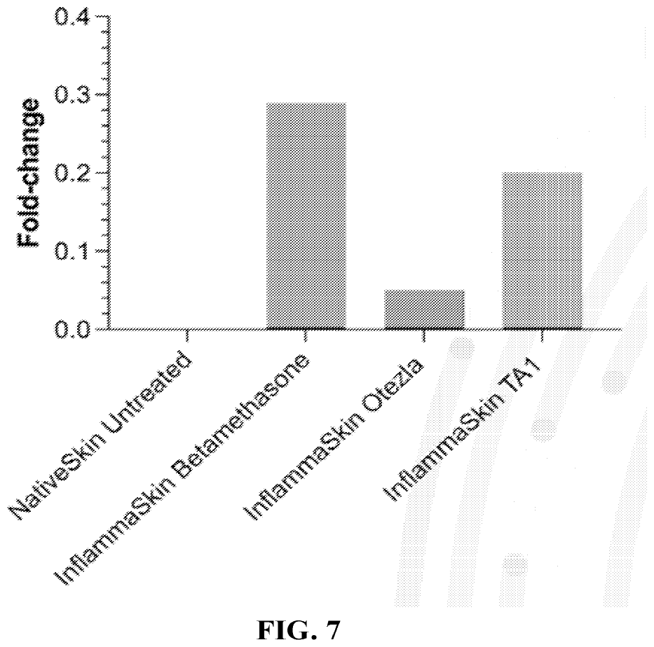

IL-17A is a cytokine upregulated both in psoriasis and in Genoskin's InflammaSkin® models. FIG. 6 shows IL-17A expression following treatment with Betamethasone, 10-15 mg Otezla® (Apremilast), or SCFAs comprising butyric acid and propionic acid. FIG. 7 shows IL-17A fold change from untreated samples following treatment with Betamethasone, 10-15 mg Otezla® (Apremilast), or SCFAs comprising butyric acid and propionic acid. IL-17A was expressed as expected in untreated InflammaSkin® models. While all three treatments, betamethasone, Otezla, and SCFA significantly decreased IL-17A compared to the untreated InflammaSkin® controls, no statistical significance was observed between the three different treatments. IL-17A levels were decreased by about 70% for betamethasone, by about 95% for Otezla, and by about 80% for SCFA. Although not statistically significant, both Otezla and SCFA were more efficacious at reducing this cytokine level in the InflammaSkin® models than the betamethasone treatment.

TNF-α is a cytokine upregulated in psoriasis and slightly in Genoskin's InflammaSkin® models (Jardet et al, 2020 Exp Derm).

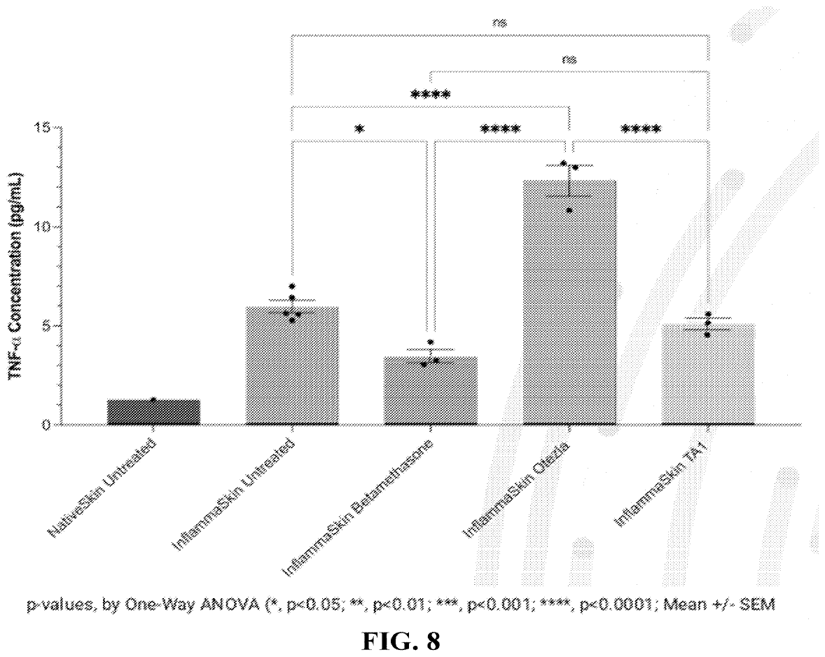

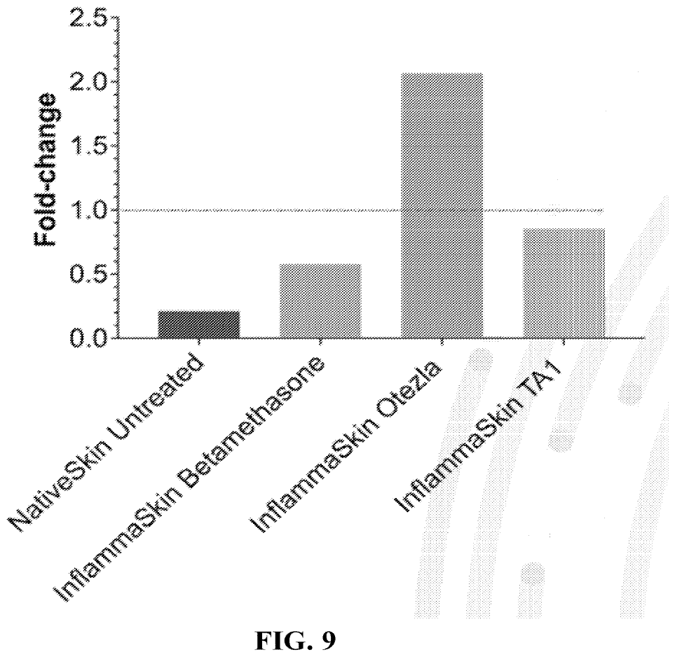

FIG. 8 shows TNF-α expression following treatment with Betamethasone, 10-15 mg Otezla® (Apremilast), or SCFAs comprising butyric acid and propionic acid. FIG. 9 shows TNF-α fold change from untreated samples following treatment with Betamethasone, 10-15 mg Otezla® (Apremilast), or SCFAs comprising butyric acid and propionic acid. TNF-α was expressed as expected in untreated InflammaSkin® models. Betamethasone significantly decreased the levels of TNF-α (by ~40%) in comparison to untreated InflammaSkin® models. SCFAs slightly decrease TNF-α (by ~15%) levels. Rather than decreasing TNF-α levels, as expected (Schafer, 2012, Biochemical Pharmacology), Otezla appears to have significantly increased the release of TNF-α, by almost 2 fold, in the InflammaSkin® models.

### Example 2: Treatment of Skin Disorders with Short Chain Fatty Acids-Assessment of Th17 Cytokine Response

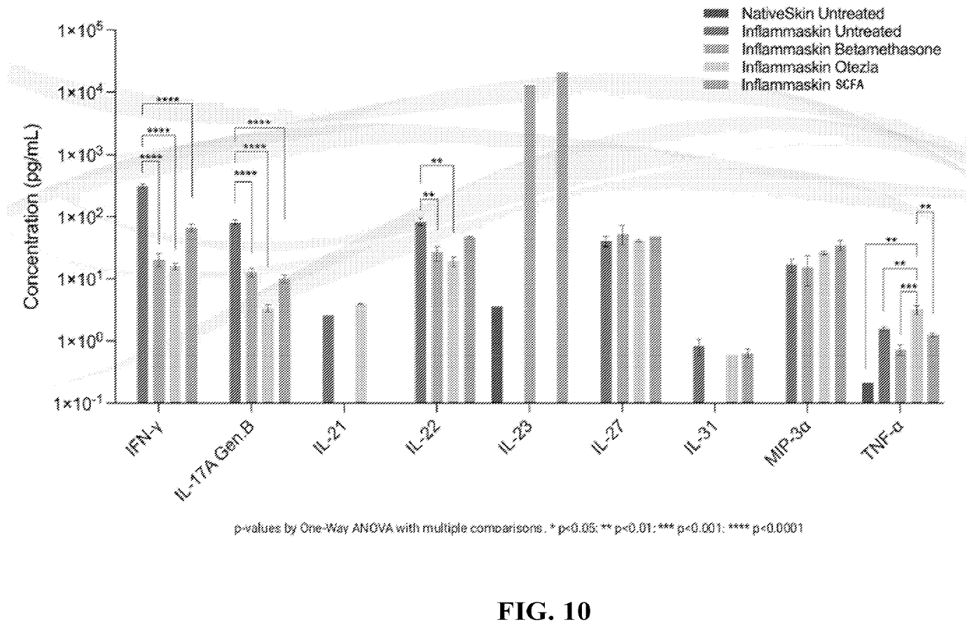

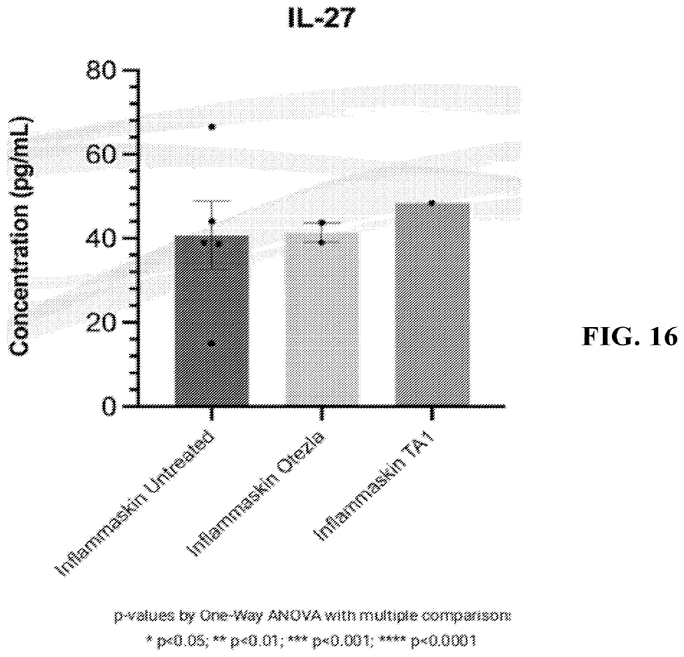

Various assays were designed and tested in an ex vivo human skin psoriasis model (e.g., Genoskin's InflammaSkin® model) as shown and described for FIGS. 10-20. Assays included evaluations of various cytokines (e.g., IL-17A Gen B, IL-21, IL-22, IL-23, IFN-γ, and TNF-α).

PSO-InflammaSkin® models were cultured and treated as described in Example 1. In these experiments, the SCFAs administered were 45 μg/mL of Ca-propionate and 1 mM mixture of salts (Ca-butyrate and Mg-butyrate).

### Cytokine Analysis—Multiplex Assay

Human pro-inflammatory cytokines released in the supernatant were quantified using the V-PLEX Human IL-17A Kit (MesoScale Discovery, reference: K151RFD-1) and V-PLEX Human custom Pro-inflammatory. Plate 1 comprised Proinflammatory Panel 1 (human) kit reference K15049 (panel IFN-γ and TNF-α). Plate 2 comprised TH17 Panel 1 (human) kit reference K15049 (panel IL-17A Gen B, IL-21, IL-22, IL-23, IL-27, IL-31, MIP-3α).

Samples were diluted four times for plate 2 and two times for plate 1. Samples and standards were assessed in duplicate. Plates were read on MesoScale Discovery Quickpleax SQ 120 plate reader and values expressed in pg/mL.

A statistical analysis was performed. Each condition was compared to the untreated InflammaSkin® condition. First, the distribution's normality of the two compared group was assessed. If the two distributions were not normal, a non-parametric Mann-Whitney-Wilcoxon test was performed. If the two distributions were normal, the homogeneity of variances was assessed using a Fisher test on the variances. If the variances were homogeneous, a Student t-test was performed. If variances were not homogeneous, a Welch test was performed

The TH17 panel plus IFN-γ and TNFα V-plex kits from MSD (IL-17A Gen.B, IL-21, IL-22, IL-23, IL-27, IL-31, MIP-3α, TNFα, and IFN-γ) were used to assess cytokine/chemokine release into the culture media. IL-23 was exogenously added to the culture media to maintain inflammation in the InflammaSkin® models over the course of the experiment. Therefore many of the values were above the detection limits and were not plotted.

FIG. 10 shows cytokine expression following treatment with Betamethasone, Otezla® (Apremilast), or SCFAs comprising butyric acid and propionic acid. Betamethasone largely reduced most of the cytokines/chemokines assessed: IFN-γ, IL-17A, IL-22, and TNFα. Only 1 replicate out of three reduced IL-23 levels enough for IL-23 to be within the limit of detection. Betamethasone treatment of InflammaSkin® models increased IL-27 cytokine secretion by about 30%.

FIGS. 10-19 shows cytokine expression following treatment with Otezla® (Apremilast), or SCFAs comprising butyric acid and propionic acid.

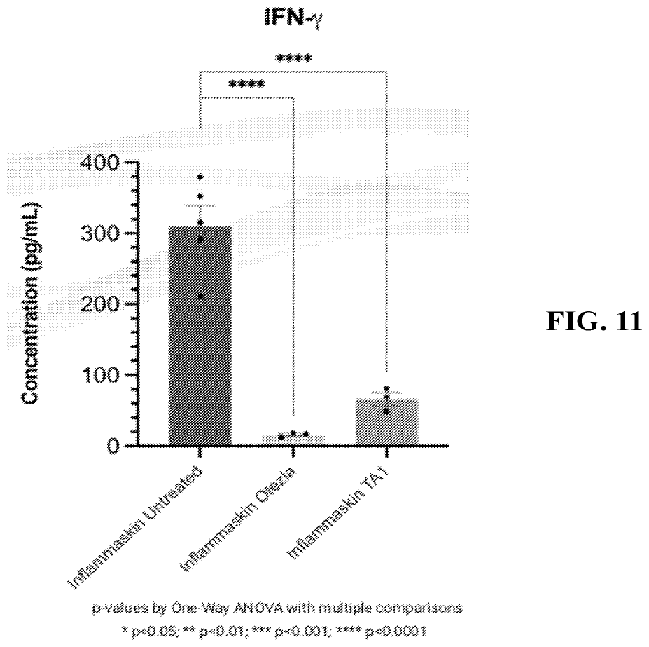

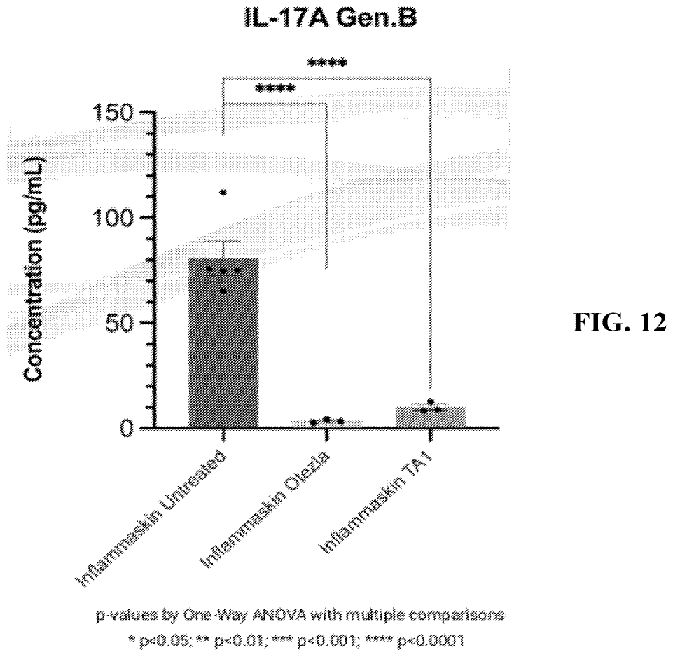

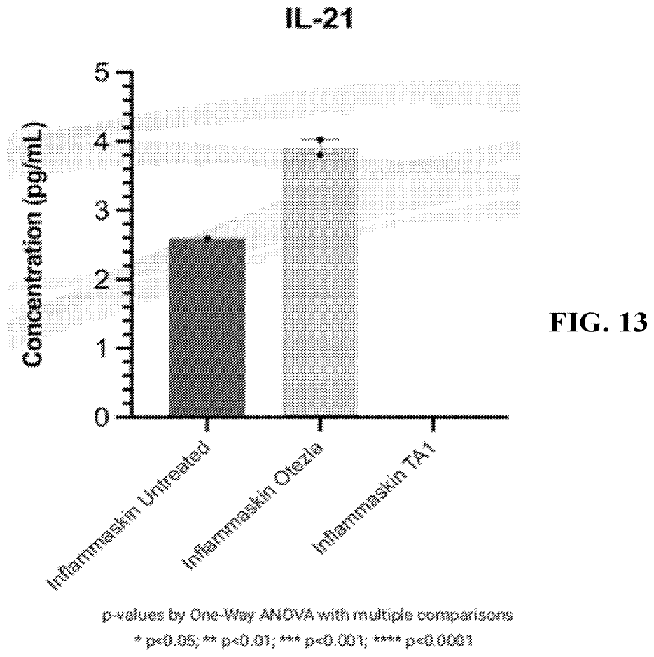

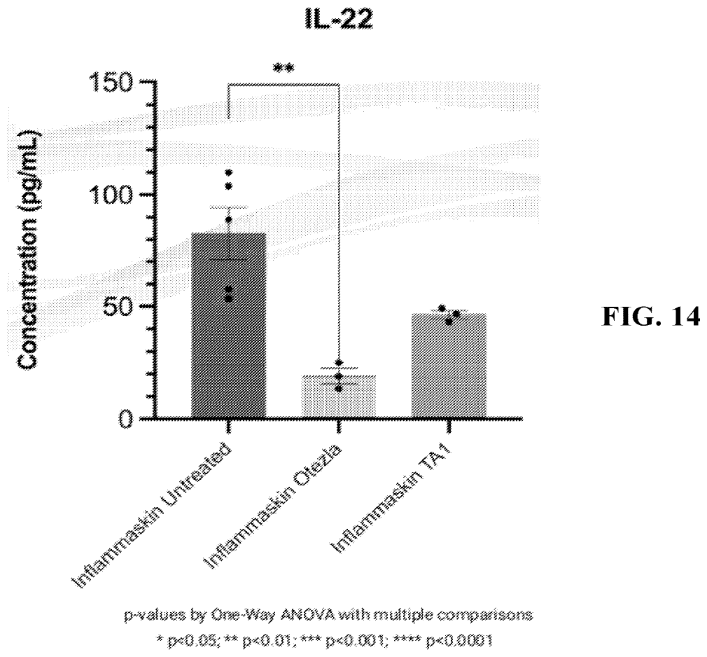

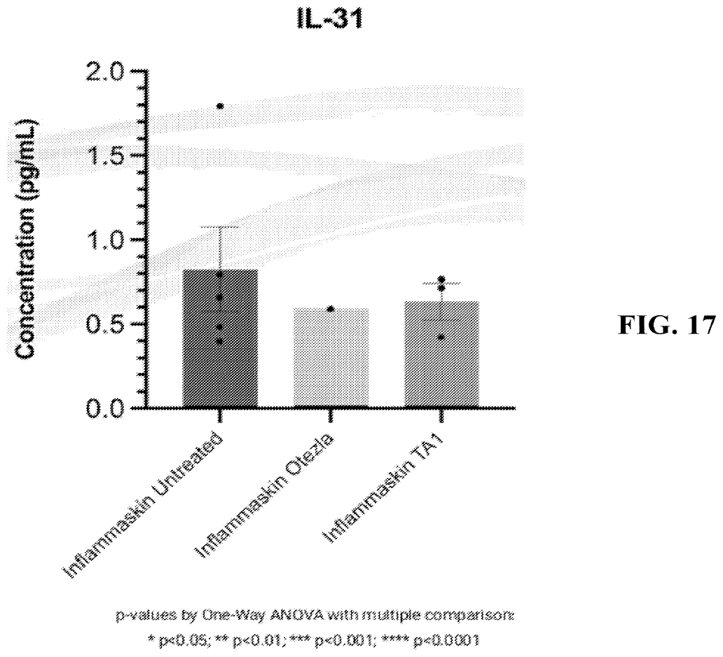

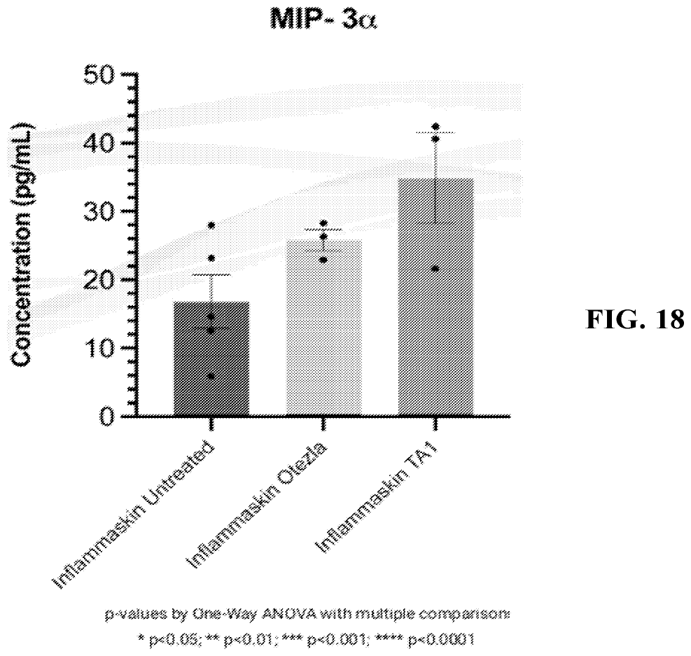

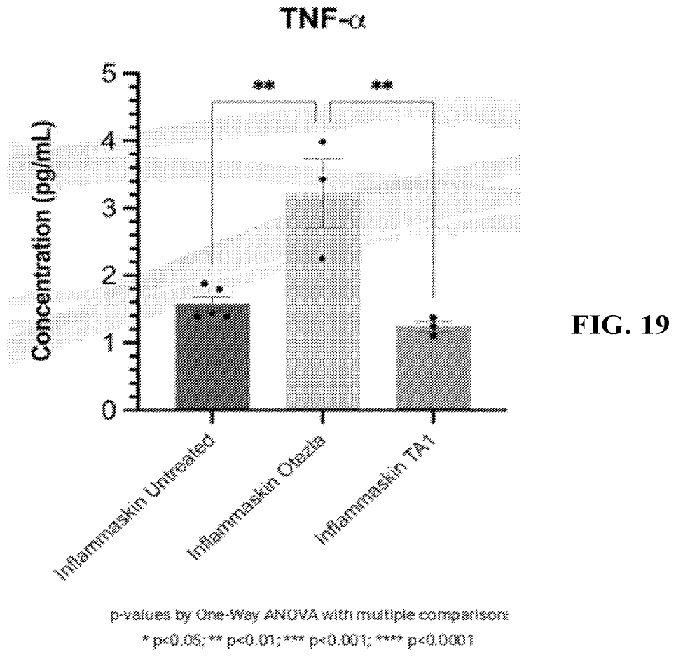

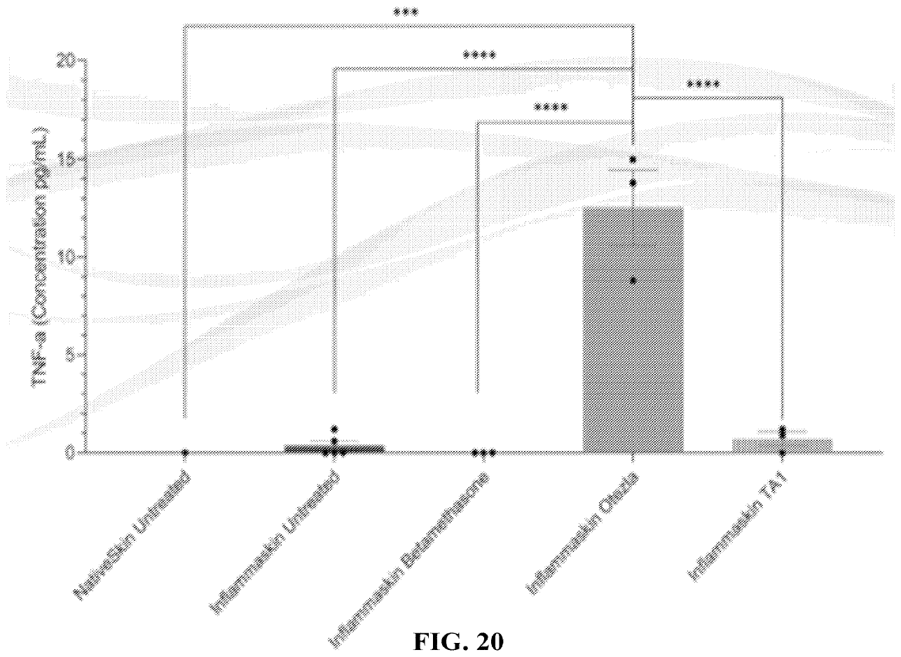

Otezla reduced some key cytokines associated with TH17 inflammation. Otezla significantly reduced IFN-γ (FIG. 10 and FIG. 11), IL-22 (FIG. 10 and FIG. 14), and IL-17A (FIG. 10 and FIG. 12) secretion from InflammaSkin® models. Otezla also reduced IL-31 (FIG. 10 and FIG. 17), but it was not significantly different from untreated InflammaSkin® models. Otezla reduced IL-17A levels more so than betamethasone treatment (FIG. 10). Otezla significantly increased the secretion of TNF-α from InflammaSkin® models (FIG. 10 and FIG. 19). However, TNFα was about 1 μg/mL in untreated InflammaSkin® models, and about 2 μg/mL (by MSD) or 13 μg/mL (by ELISA; FIG. 20). The cytokines such as IFN-γ, IL-22, and IL-17A, which were closer to 100 μg/mL in untreated InflammaSkin® models, were drastically reduced by Otezla. Besides TNFα, Otezla also increased the secretion of MIP-3a (FIG. 10 and FIG. 18), and IL-21 (FIG. 10 and FIG. 13), by about 50% compared to the untreated InflammaSkin® models.

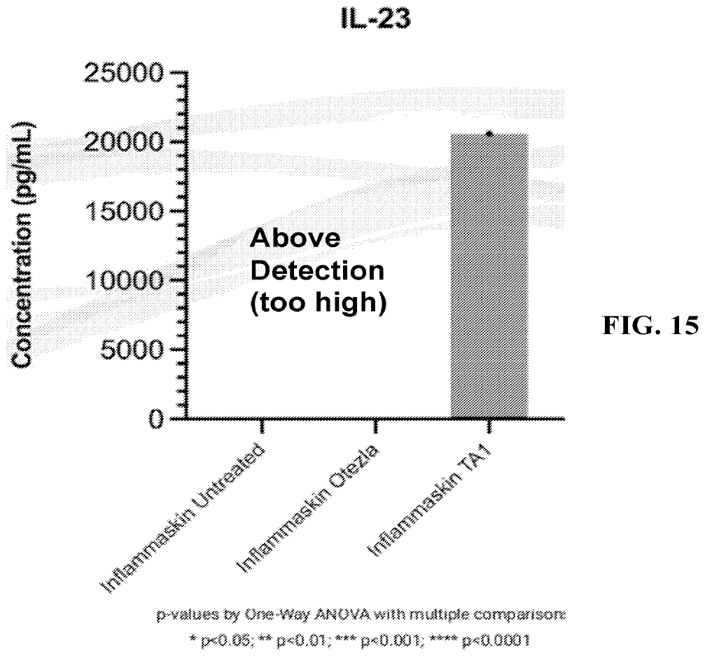

SCFA reduced IFN-γ (FIG. 10 and FIG. 11), IL-17A (FIG. 10 and FIG. 12), IL-22 (FIG. 10 and FIG. 14), IL-31 (FIG. 10 and FIG. 17) and TNFα (FIG. 10 and FIG. 19). SCFA treatment of InflammaSkin® models did not result in as much of a decrease in IFNγ, IL-17A, or IL-22, as Otezla. Only 1 replicate out of three reduced IL-23 levels enough for IL-23 to be within the limit of detection (FIG. 10 and FIG. 15). TAI also did not cause an increase in TNFα like Otezla in the InflammaSkin® models (FIG. 10 and FIG. 19). MIP-3a was increased by about 2-fold upon SCFA treatment of InflammaSkin® models (FIG. 10 and FIG. 18).

### Example 3: Combination Treatment of Skin Disorders Using Short Chain Fatty Acids

Assays are performed to evaluate the efficacy of combination therapy of at least one SCFA in combination with at least one second compound for treating skin disease and disorders, including psoriasis.

The combination of at least one SCFA with at least one second compound provides an effective treatment for one or more skin disorders. Additional compounds that are contemplated for use include a PDE4 inhibitor, an anti-inflammatory compound, a disease-modifying antirheumatic drug (DMARD), an immunosuppressant, a biologic agent, a Cox-2 inhibitor, Apremilast or a combination thereof, and/or another agent. Non-limiting examples of these and other compounds useful in the combination are disclosed herein.

### Combination of SCFA and a PDE4 Inhibitor (e.g., Apremilast)

Combination of SCFA treatment with apremilast: the daily oral dose is about 1 to about 2 g of butyric acid or a pharmaceutically-acceptable salt thereof, for example, sodium butyrate, about 100 mg propionic acid or a pharmaceutically-acceptable salt thereof, for example, sodium propionate, about 10 to about 15 mg apremilast, about 10 to about 20 mg of a source of magnesium, for example, magnesium chloride, about 80 to about 100 IU Vitamin D3, and about 50 to about 100 IU Vitamin E (d-alpha-tocopherol acetate).

For psoriasis, combination with topical ointments is important. The daily oral dose of SCFAs are used accompanied with applying ointment consisting of: ⅖ part clobetazol (0.05%), ⅕ part calcipotriene (vit D, 0.005%), ⅕ part salicylic acid (10%), ⅓ part vit E (0.5%). All these components (separately) are used as topical treatment.

Efficacy of combination therapy are assessed in comparison to the efficacy of monotherapy for each test compound. Assays to determine synergy of SCFAs in combination with a PDE4 inhibitor are also evaluated.

### Example 4: Treatment of Skin Disorders with Short Chain Fatty Acids

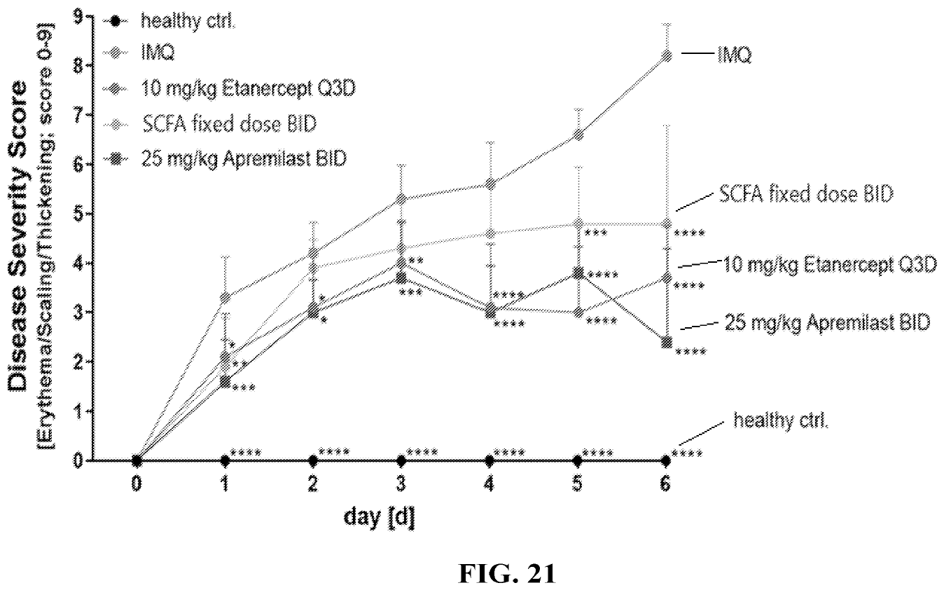

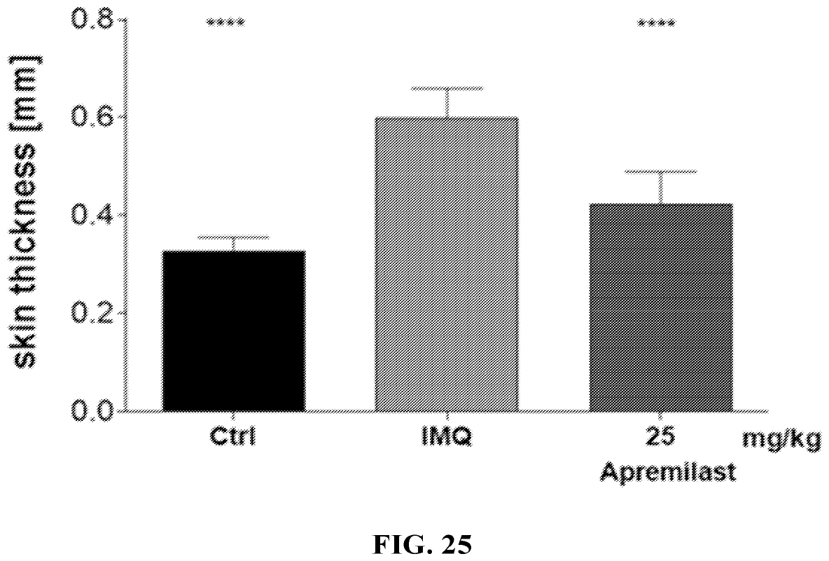

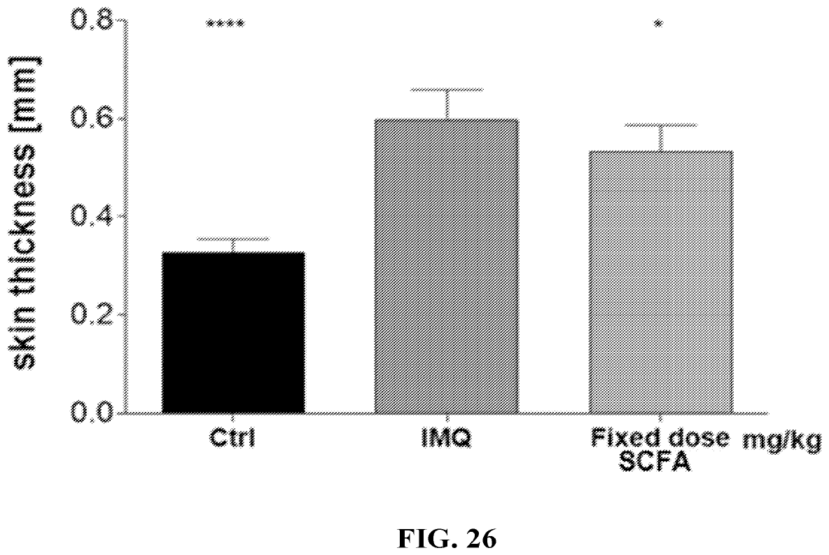

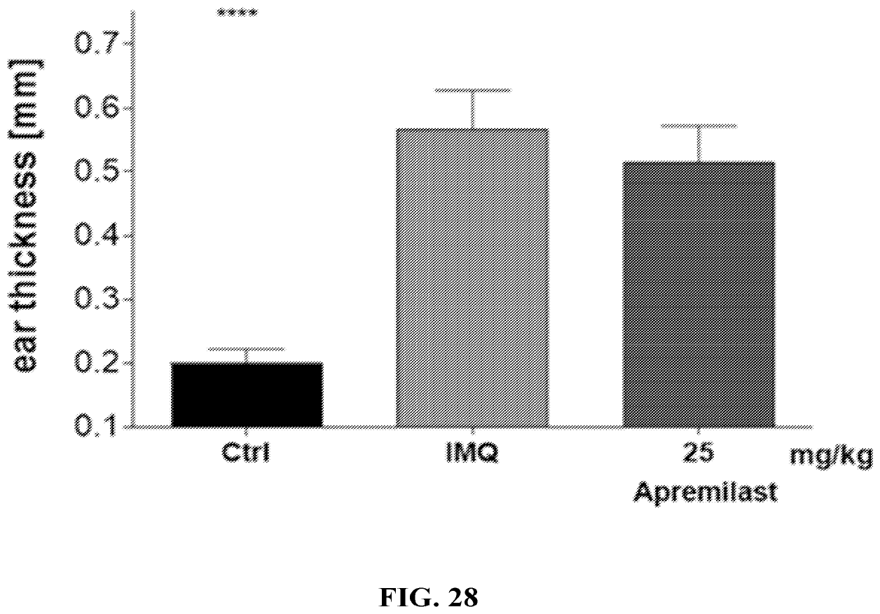

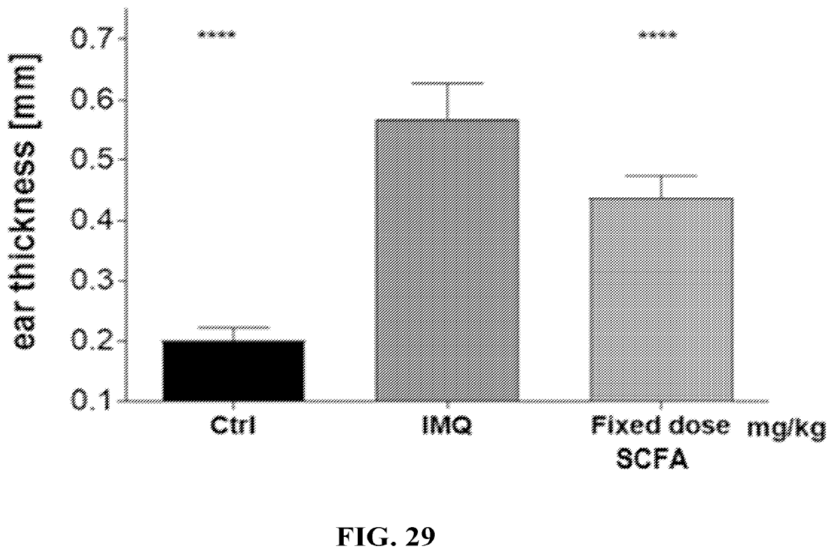

Various assays were designed and tested in the imiquimod (IMQ)-induced psoriasis model in mice as shown and described for FIGS. 21-31. The anti-inflammatory activity of PDE4-inhibitor Apremilast and SCFA were evaluated in a mouse imiquimod (IMQ)-induced psoriasis model.

### Imiquimod (IMQ)-Induced Psoriasis Model

Topical application of IMQ, a TLR7/8 ligand and potent immune activator induced and exacerbated psoriasis in mice. The mouse IMQ-induced psoriasis (IMQ Ps) model was initiated by a repeated topical application of Aldara© cream (containing 5% IMQ) over 6 consecutive days. Female Balb/c mice (9 weeks of age at reception, ordered with 20 g, Charles River, Sulzfeld, Germany; n=10 mice/group, exception healthy controls n=4) received daily a topical application of 62.5 mg of commercially available IMQ cream (5% Aldara©) on the beforehand (day −1) shaved (~2 cm×3 cm), depilated (Pilka© cream) back skin, and 7 mg on the outer right ear skin for 6 consecutive days, translating in a daily dose of approximately 3.5 mg IMQ. Healthy control mice were included in the study and were treated with vehicle cream (Vaseline; Bombastus-Werke AG) only instead of IMQ. Additionally, an IMQ disease control group was also included.

Before starting treatment on the day of disease induction (day 0), mice were randomized based on body weight to the respective group. To evaluate the anti-inflammatory efficacy of the two test compounds in mouse IMQ psoriasis, mice were treated twice daily (BID) by oral gavage either with 25 mg/kg of Apremilast (in 0.5% CMC/0.025% Tween80 in sterile water; m/m/v) or with fixed dose of SCFA (in ddH2O) starting on day 0.

In these experiments, the SCFAs administered were 120 mg/kg/day of Ca-propionate and 2,146 mg/kg/day mixture of salts (Ca-butyrate and Mg-butyrate).

IMQ psoriasis is characterized by rapid induction of skin inflammation with pathologic and histologic resemblance to human psoriasis, including skin erythema, skin scaling and epidermal thickening (acanthosis), accompanied with immune cell infiltration.

### Psoriasis Severity Score

Disease Severity Score was evaluated on a daily base starting with the induction of psoriasis by IMQ application (day 0) until the end (day 6) of the study. The psoriasis severity on the back skin of the mice was evaluated by using a modified scoring system developed by van der Fits et al., based on the clinical PASI (psoriasis area and severity index) and takes skin erythema, scaling and skin thickening into account. Scoring was performed by the same lab technician throughout the experiment. Each parameter was scored independently on a scale from 0 to 3.

TABLE 1
Psoriasis disease severity score

| Score | Erythema | Skin Scaling | Skin Thickening | |
|---|---|---|---|---|
| 0 | none | none | 0-0.07 | mm |
| 1 | slight | slight | 0.08-0.14 | mm |
| 2 | moderate | moderate | 0.15-0.21 | mm |
| 3 | marked | marked | >0.22 | mm |

The added-up score (erythema plus scaling plus thickening), named disease severity score (scale 0-9), served then as marker for the severity of back skin inflammation. In addition, ear thickness of the right ear was determined daily by using an automatized caliper (Bayer AG, Germany).

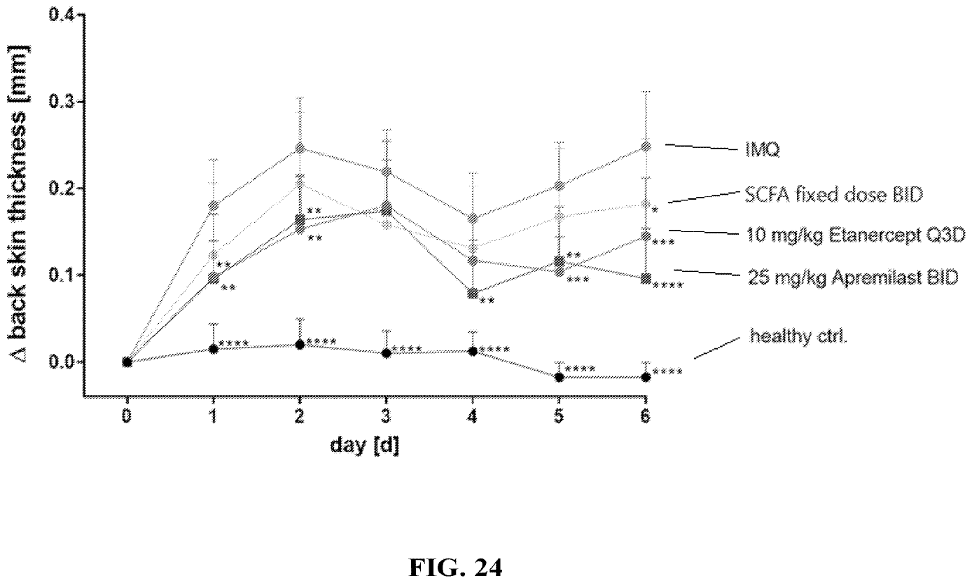

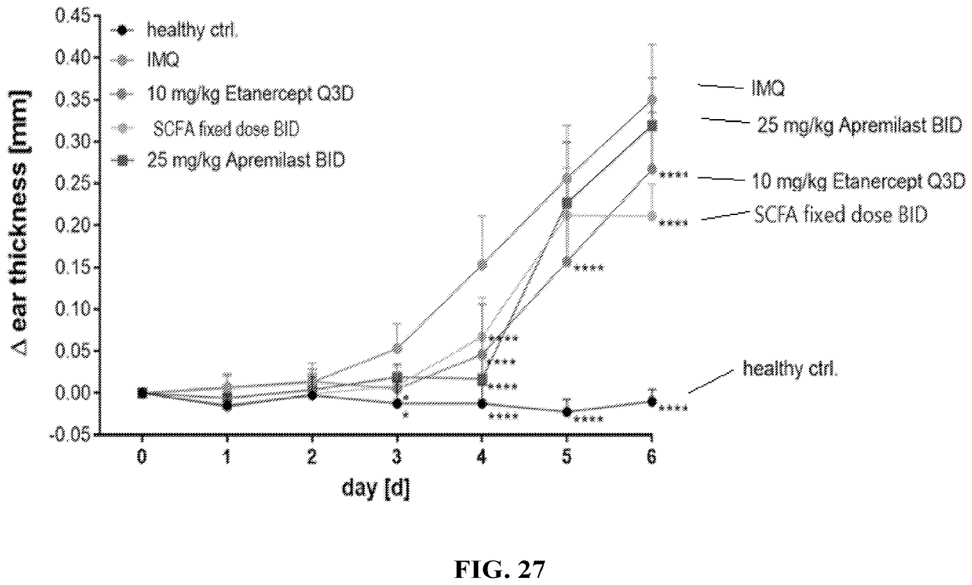

During the study, parameters including the disease severity score (erythema, scaling, thickening) were assessed at psoriatic back skin and the extent of ear thickening of the IMQ-treated right ear were evaluated. All IMQ control mice developed a psoriasis phenotype at the back skin over the course of the study and exhibited thereafter an increased disease severity score (FIGS. 21-23), augmented back (FIGS. 24-26), as well as ear thickness (FIGS. 27-29).

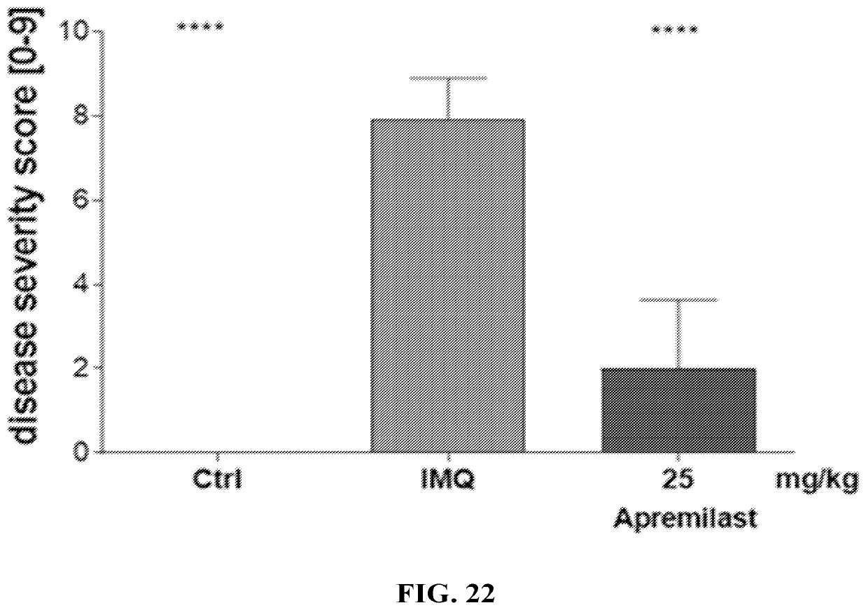

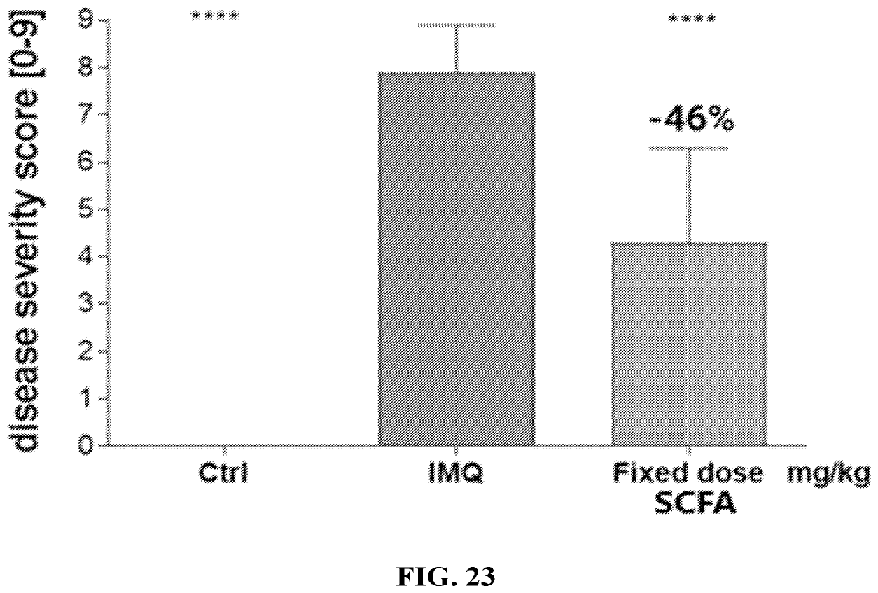

Treatment with 50 mg/kg/day of PDE4 inhibitor Apremilast significantly inhibited the psoriasis severity in IMQ-treated mice throughout the duration of the experiment and showed thereby efficacy comparable to SoC (standard of care) Etanercept, which served as a technical control (FIG. 21 and FIG. 22). Fixed dose of SCFA also significantly reduced the disease severity score at day 5 and day 6 (FIG. 21 and FIG. 23), accompanied by inhibitory effects on ear thickness throughout the study duration (FIGS. 27, 29, and 30).

Both compounds reduced skin erythema, scaling and skin thickening as assessed by the disease severity score (FIGS. 21-23), and separate determination of back/ear skin thickness (FIGS. 24-26 and FIGS. 27-28).

### Transepidermal Water Loss (TEWL)

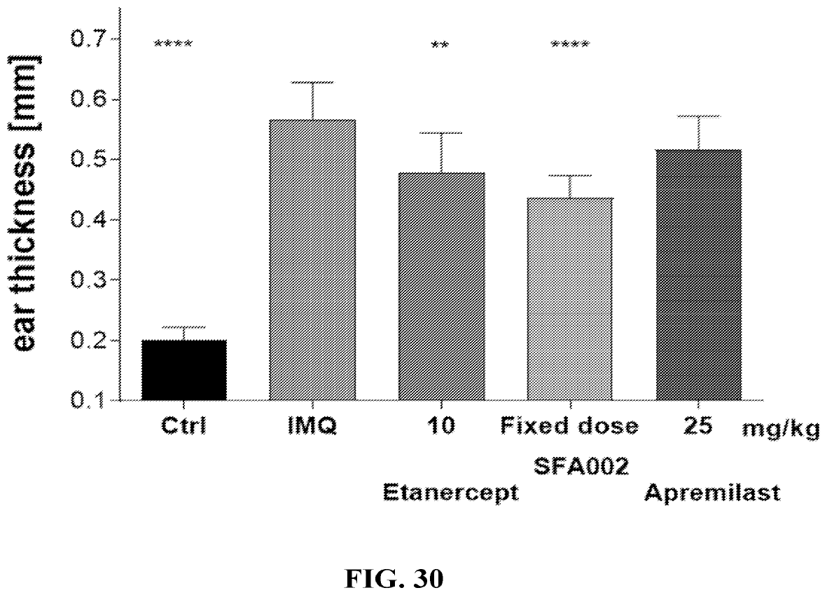

Physiological parameter transepidermal water loss (TEWL; in g/m²/h) was tested and evaluated on back skin at day 5 by a Tewameter (TM300, Courage and Khazaka, Cologne, Germany) during the IMQ psoriasis experiment. TEWL is a common examined parameters for the assessment of a disturbed skin function present in psoriatic skin. TEWL may be used a diagnostic marker in the clinics to accurately assess the disease severity in psoriasis. Physiological parameter TEWL showed an increase in transepidermal water loss on day 5 in psoriatic back skin of IMQ control mice compared to healthy skin of control mice (FIG. 30).

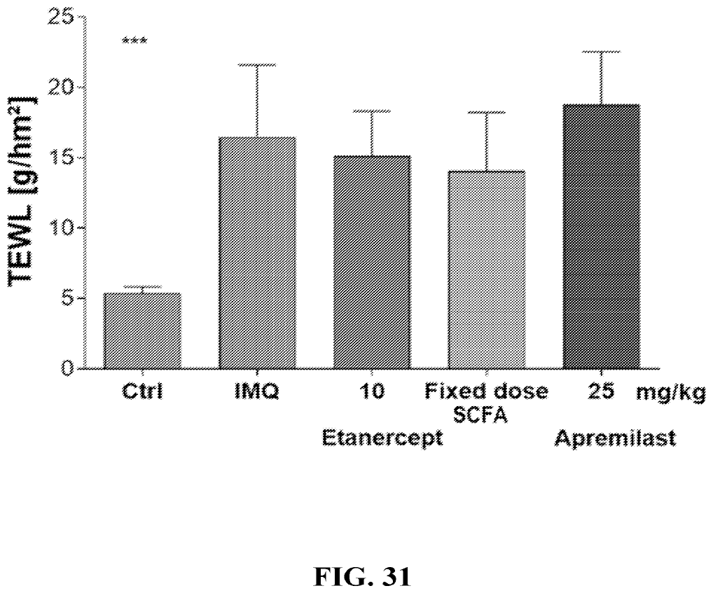

Although, both test compounds and technical control Etanercept did not show significant effects on the newly tested parameter of transepidermal water loss (TEWL), a trend in reduction by SCFA could be observed (FIG. 31).

### Body Weight

Body weight was determined daily in all mice over the whole period of each study. Oral treatment with either Apremilast (25 mg/kg BID) or SFA002 (fixed dose BID) started with the disease induction on day 0. Etanercept (10 mg/kg Q3D s.c.) served as a technical control. Healthy control and IMQ control group received no placebo treatment.

As known, IMQ induces within the first 2 to 3 days a slight body weight loss of approximately 10% (FIG. 30). Both test compounds showed no effect on body weight loss by treatment with Apremilast and SCFA in psoriatic skin from IMQ psoriasis mice (FIG. 31). With regards to observations of animal well-being, SCFA was well tolerated during the study duration, whereas mice treated with 50 mg/kg/day of Apremilast showed reduced activity, ruffled coat and were slightly hunched.

On day 6, following the assessment of the disease severity score and evaluation of ear thickness, animals were sacrificed, blood was drawn from the vena cava and the right ear of each mouse was collected for potential ex vivo analysis of inflammation markers (e.g. cytokines, elastase activity). Ears were frozen in liquid nitrogen and stored at −80° C. Additionally, punch biopsies (ø8 mm, SmithKline Beecham Ltd, UK) from back skin were taken and also frozen in liquid nitrogen and stored at −80° C.

The statistical analysis of the data was conducted by using the GraphPad PRISM software. Here, a one-way analysis of variance (ANOVA) and a pair-wise comparison to the IMQ disease control group (using the Dunnett's test) as a multiple comparison procedure was performed. Statistical significance was delineated by p value (*, p<0.05; **, p<0.01; ***, p<0.005; **** p<0.001).

### Example 5: Treatment of Skin Disorders with Short Chain Fatty Acids

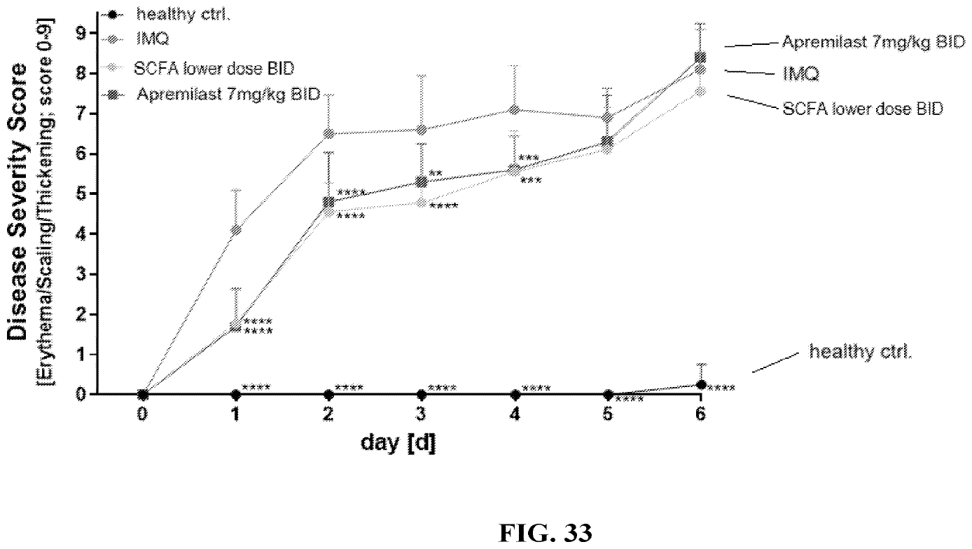

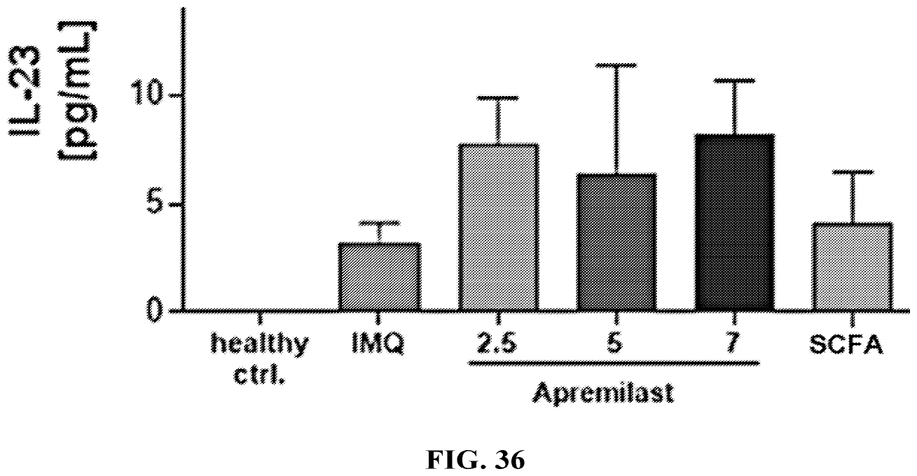

Various assays were designed and tested in the imiquimod (IMQ)-induced psoriasis model in mice as shown and described for FIGS. 33-37. The anti-inflammatory activity of PDE4-inhibitor Apremilast and SCFA were evaluated in a mouse imiquimod (IMQ)-induced psoriasis model.

### Imiquimod (IMQ)-Induced Psoriasis Model

A mouse IMQ-induced psoriasis (IMQ Ps) model was prepared as described in Example 4.

Before starting treatment on the day of disease induction (day 0), mice were randomized based on body weight to the respective group. To evaluate the anti-inflammatory efficacy of the two test compounds in mouse IMQ psoriasis, mice were treated twice daily (BID) by oral gavage either with 2.5 mg/kg, 5 mg/kg, and/or 7 mg/kg of Apremilast (in 0.5% CMC/0.025% Tween80 in sterile water; m/m/v) or with fixed dose or lower dose of SCFA (in ddH2O) starting on day 0.

In these experiments, the fixed dose of SCFAs administered was 120 mg/kg/day of Ca-propionate and 2,146 mg/kg/day mixture of Ca-butyrate and Mg-butyrate, and the lower dose of SCFAs administered was 120 mg/kg/day of Ca-propionate and 1,100 mg/kg/day mixture of salts (Ca-butyrate and Mg-butyrate).

### Psoriasis Severity Score

Disease Severity Score was evaluated on a daily base as described in Example 4.

During the study, parameters including the disease severity score (erythema, scaling, thickening) were assessed at psoriatic back skin

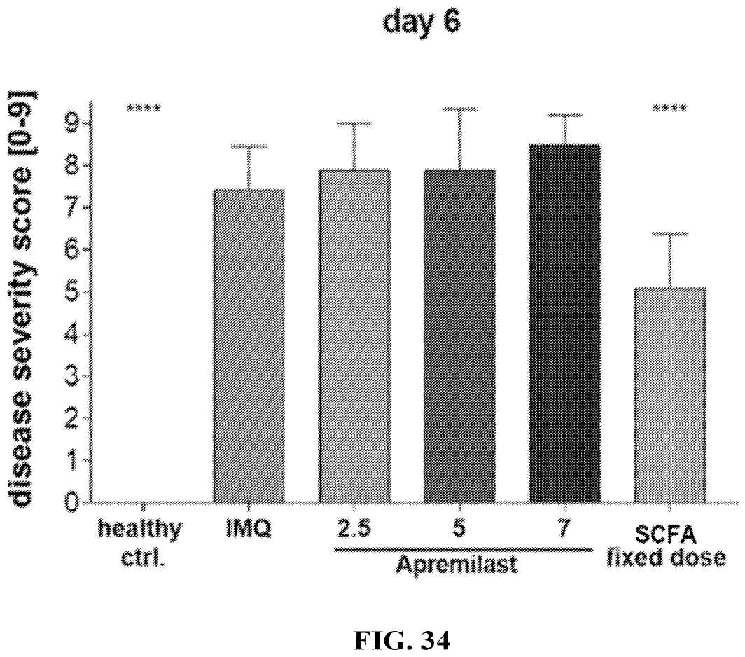

Treatment with 10 mg/kg/day and 14 mg/kg/day of PDE4 inhibitor Apremilast inhibited the psoriasis severity in IMQ-treated mice within the first four days of the study, but anti-inflammatory effects were not maintained until the end of the study at day 6 (FIG. 33 and FIG. 34). Treatment with lower dose of SCFA inhibited the psoriasis severity in IMQ-treated mice within the first four days of the study, but anti-inflammatory effects were not maintained until the end of the study at day 6 (FIG. 33). However, treatment with fixed dose of SCFA significantly reduced the disease severity score throughout the study, including at day 5 and day 6 (FIG. 34).

### Cytokine Analysis—Multiplex Assay

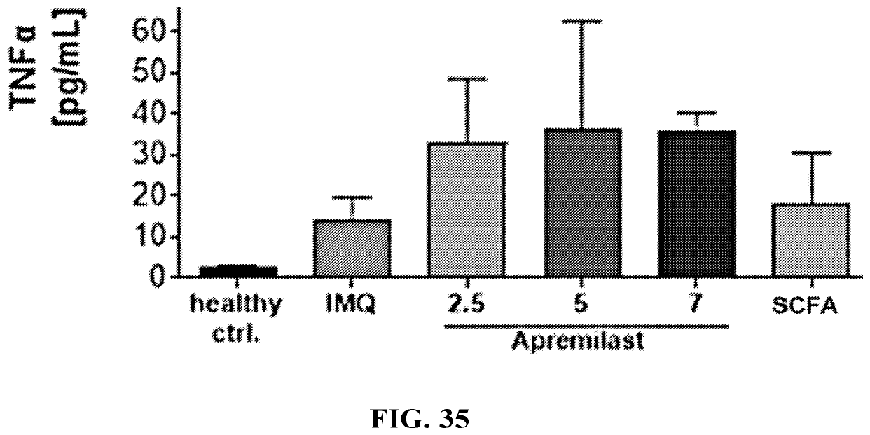

Human pro-inflammatory cytokines (e.g., TNFα, IL-17A and IL-23) of each mouse were measured by multiplex ELISA in back skin biopsies collected on day 6 after IMQ psoriasis study (FIGS. 35-37).

### Example 6: Combination Treatment of Skin Disorders Using Short Chain Fatty Acids

Various assays are designed and tested in the imiquimod (IMQ)-induced psoriasis model in mice.

### Imiquimod (IMQ)-Induced Psoriasis Model

A mouse IMQ-induced psoriasis (IMQ Ps) model is prepared as described in Example 4.

Before starting treatment on the day of disease induction (day 0), mice are randomized based on body weight to the respective group. To evaluate the anti-inflammatory efficacy of the two test compounds in mouse IMQ psoriasis, mice are treated twice daily (BID) by oral gavage either with 2.5 mg/kg, 5 mg/kg, and/or 7 mg/kg of Apremilast (in 0.5% CMC/0.025% Tween80 in sterile water; m/m/v) or with lower dose of SCFA (in ddH2O) starting on day 0. Healthy and IMQ disease controls are orally treated in parallel with vehicle only. All treatments start with first application of IMQ on day 0.

In these experiments, the fixed dose of SCFAs administered were 120 mg/kg/day of Ca-propionate and 2,146 mg/kg/day of Ca-butyrate and Mg-butyrate, and the lower dose of SCFAs administered were 120 mg/kg/day of Ca-propionate and 1,100 mg/kg/day mixture of salts (Ca-butyrate and Mg-butyrate).

### Psoriasis Severity Score

Disease Severity Score is evaluated on a daily base as described in Example 4.

During the study, the disease severity score (comprising erythema, skin scaling and thickening) resembling the psoriasis severity is assessed daily at the psoriatic back skin. Next to this, the extent of ear thickening of the IMQ-treated right ear is also evaluated daily.

The added-up score (erythema plus scaling plus thickening), named disease severity score (scale 0-9), served then as marker for the severity of back skin inflammation. In addition, ear thickness of the right ear was determined daily by using an automatized caliper (Bayer AG, Germany).

During the study, parameters including the disease severity score (erythema, scaling, thickening) are assessed at psoriatic back skin and the extent of ear thickening of the IMQ-treated right ear are evaluated.

### Body Weight

Body weight is determined daily in all mice over the whole period of each study as described in Example 4.

### Cytokine Analysis—Multiplex Assay

Human pro-inflammatory cytokines (e.g., TNFα, IL-17A and IL-23) of each mouse are measured by multiplex ELISA in back skin biopsies collected on day 6 after IMQ psoriasis study.

### Example 7: Combination Treatment of Skin Disorders Using Short Chain Fatty Acids

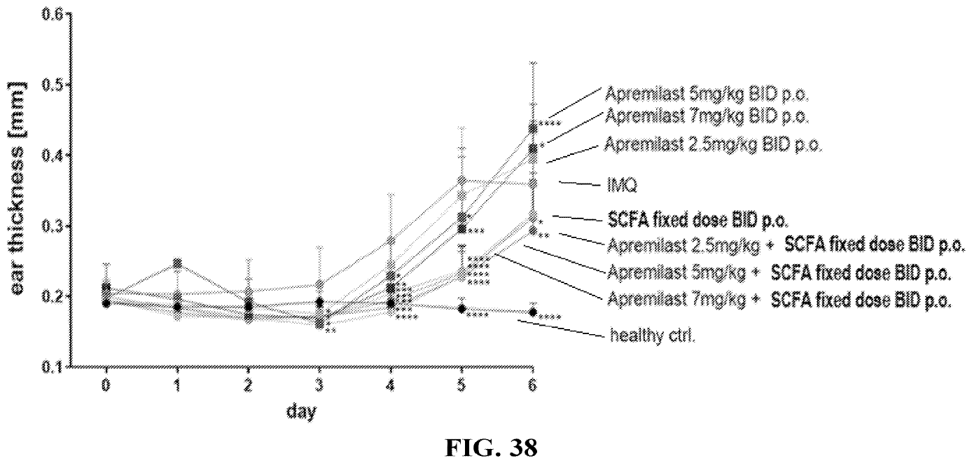

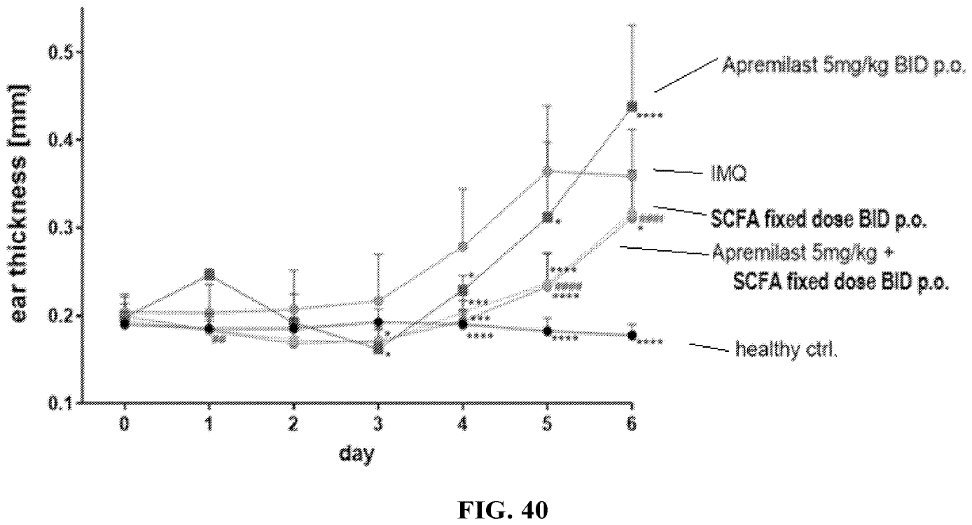

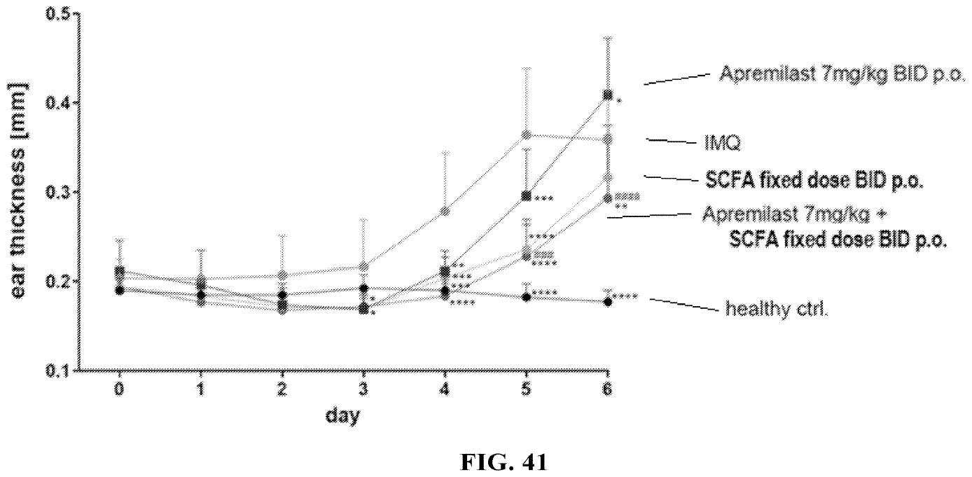

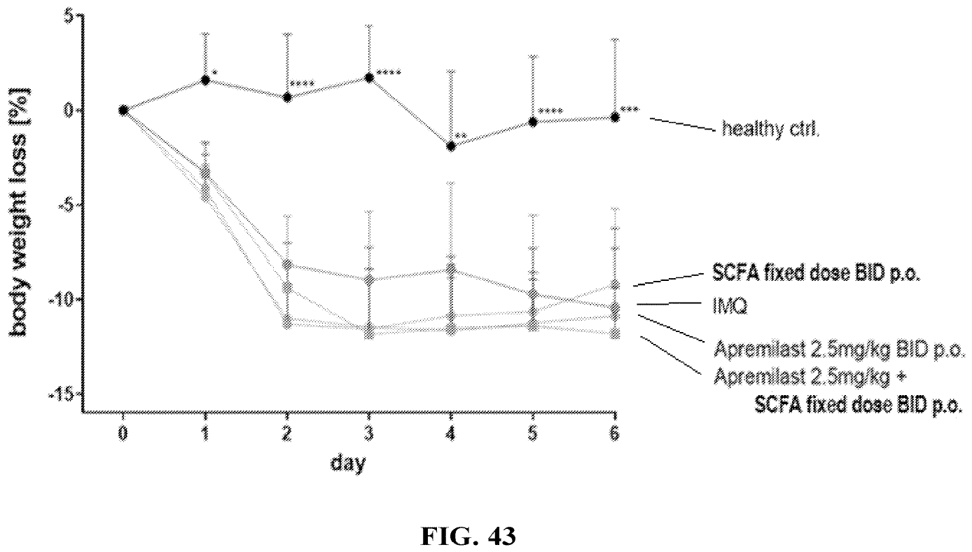

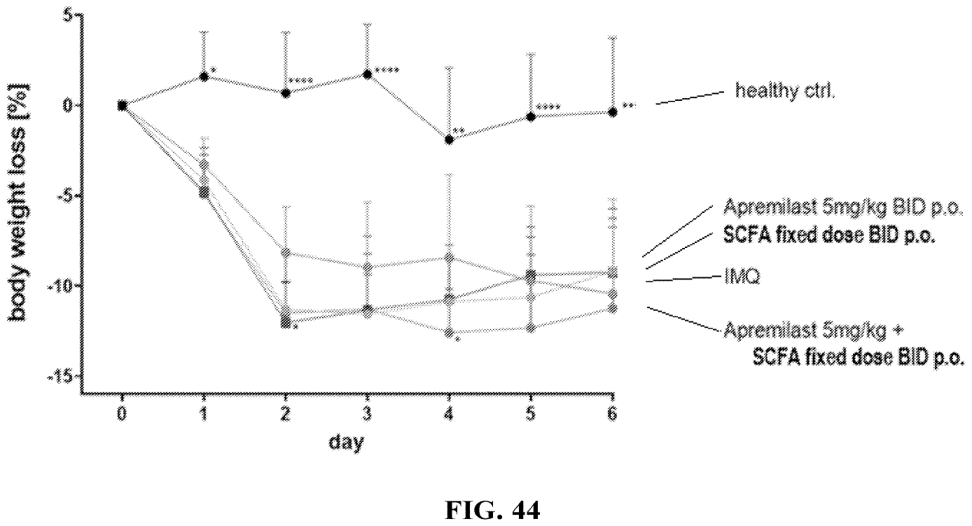

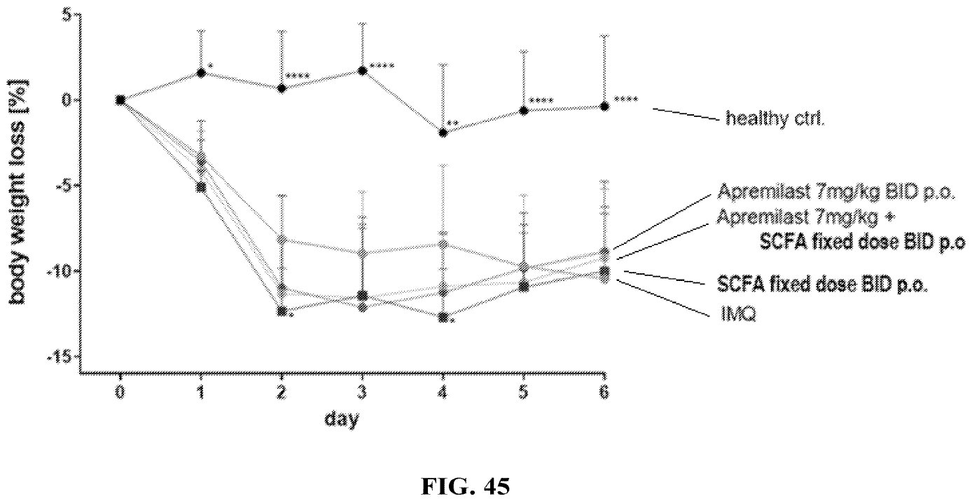

Various assays were designed and tested in the imiquimod (IMQ)-induced psoriasis model in mice as shown and described for FIGS. 38-45. The anti-inflammatory activity of PDE4-inhibitor Apremilast and SCFAs were evaluated in combination vs. monotherapy in the mouse imiquimod (IMQ)-induced psoriasis model.

### Imiquimod (IMQ)-Induced Psoriasis Model

A mouse IMQ-induced psoriasis (IMQ Ps) model was prepared as described in Example 4.

Before starting treatment on the day of disease induction (day 0), mice were randomized based on body weight to the respective group. To evaluate the anti-inflammatory efficacy of the two compounds in mouse IMQ psoriasis, mice were treated twice daily (BID) by oral gavage with 2.5 mg/kg, 5 mg/kg, or 7 mg/kg of Apremilast (in 0.5% CMC/0.025% Tween80 in sterile water; m/m/v) alone, with fixed dose of SCFA (in ddH2O) alone, with 2.5 mg/kg Apremilast in combination with fixed dose of SCFA, 5 mg/kg Apremilast in combination with fixed dose of SCFA, or 7 mg/kg of Apremilast in combination with fixed dose of SCFA. Healthy and IMQ disease controls were orally treated in parallel with vehicle only. All treatments started with first application of IMQ on day 0.

In these experiments, the SCFAs administered were 120 mg/kg/day of Ca-propionate and 1,100 mg/kg/day mixture of salts (Ca-butyrate and Mg-butyrate).

### Ear Thickening

Disease Severity Score was evaluated on a daily base starting with the induction of psoriasis by IMQ application (day 0) until the end (day 6) of the study. The extent of ear thickness of the IMQ-treated right ear was determined daily by using an automatized caliper (Bayer AG, Germany) as described in Example 4.

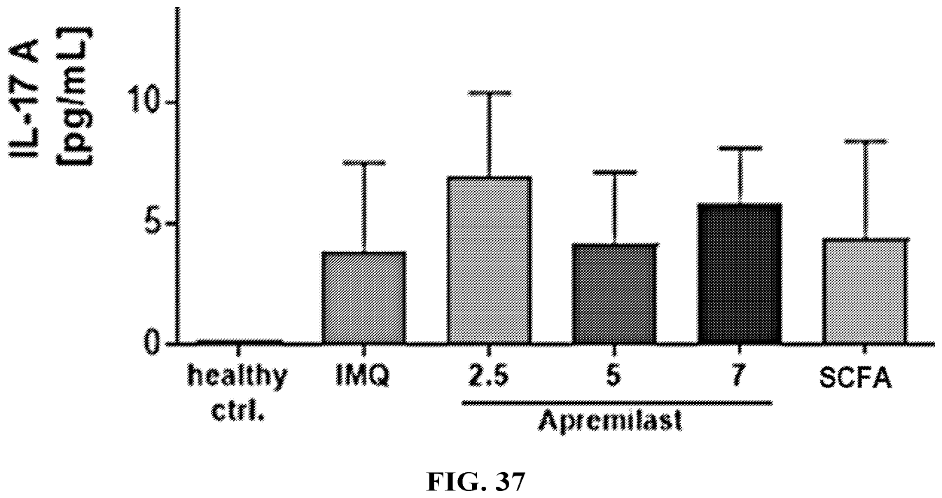

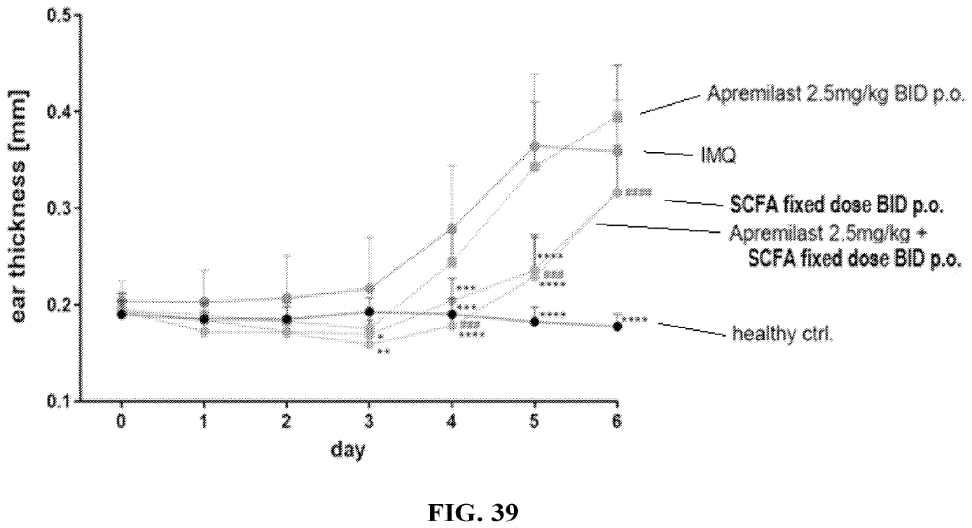

Furthermore, SCFA significantly inhibited the skin thickening of ear skin until day 5 (FIG. 37). Ear thickness was also inhibited by Apremilast's mid and high dose until day 5. Low dose of Apremilast did not show any inhibitory influence on ear thickness (FIG. 38 and FIG. 39).

Combination therapy exhibited no additive or synergistic effect with respect to reduction of ear skin thickening compared to monotherapy of corresponding doses of Apremilast or fixed dose of SCFA (FIGS. 35-41). However, a significant reduction by combination treatment compared to Apremilast monotherapies was observed for all tested doses from day 4 to day 6 with respect to inhibition of ear thickness (FIG. 38 and FIG. 39).

### Body Weight

Body weight was determined daily in all mice over the whole period of each study as described in Example 4.

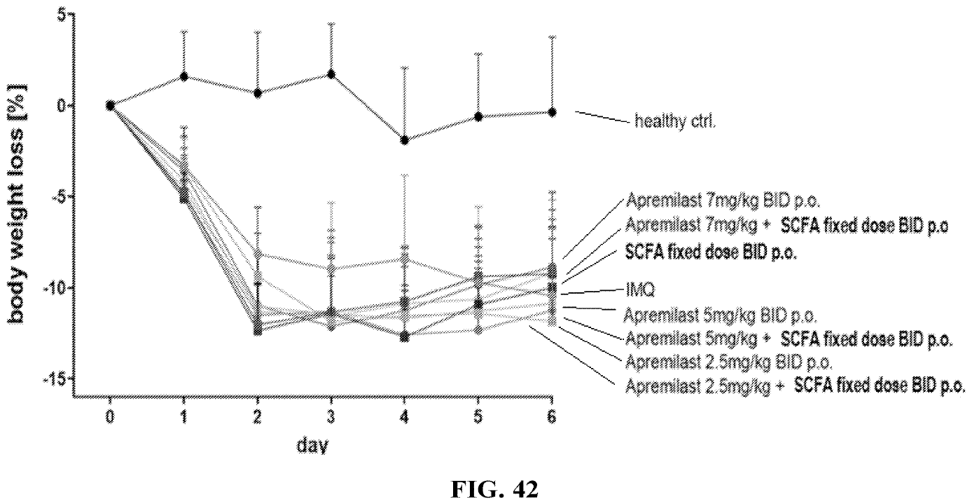

As known, IMQ induces a slight body weight loss within the first two to three days (FIGS. 42-45). Both, Apremilast and SCFA whether in combination or alone showed no positive influence on body weight loss by treatment compared to vehicle-treated IMQ disease controls (FIGS. 42-45).

With regards to observations of animal well-being, SCFA as well as all tested doses of Apremilast or combination treatments were well tolerated during the study duration.

### Example 8: Combination Treatment of Skin Disorders Using Short Chain Fatty Acids

Various assays are designed and tested in the imiquimod (IMQ)-induced psoriasis model in mice. The anti-inflammatory activity of PDE4-inhibitor Apremilast and SCFAs are evaluated in combination vs. monotherapy in the mouse imiquimod (IMQ)-induced psoriasis model.

### Imiquimod (IMQ)-Induced Psoriasis Model

A mouse IMQ-induced psoriasis (IMQ Ps) model is prepared as described in Example 4.

Before starting treatment on the day of disease induction (day 0), mice are randomized based on body weight to the respective group. To evaluate the anti-inflammatory efficacy of the two compounds in mouse IMQ psoriasis, mice are treated twice daily (BID) by oral gavage with 2.5 mg/kg, 5 mg/kg, or 7 mg/kg of Apremilast (in 0.5% CMC/0.025% Tween80 in sterile water; m/m/v) alone, with fixed dose of SCFA (in ddH2O) alone, with 2.5 mg/kg Apremilast in combination with fixed dose of SCFA, 5 mg/kg Apremilast in combination with fixed dose of SCFA, or 7 mg/kg of Apremilast in combination with fixed dose of SCFA. Healthy and IMQ disease controls are orally treated in parallel with vehicle only. All treatments start with first application of IMQ on day 0.

In these experiments, the SCFAs administered were 120 mg/kg/day of Ca-propionate and 1,100 mg/kg/day mixture of salts (Ca-butyrate and Mg-butyrate).

### Psoriasis Severity Score

Disease Severity Score is evaluated on a daily base as described in Example 4.

During the study, the disease severity score (comprising erythema, skin scaling and thickening) resembling the psoriasis severity is assessed daily at the psoriatic back skin. Next to this, the extent of ear thickening of the IMQ-treated right ear is also evaluated daily

All IMQ control mice develop a psoriasis phenotype at the back skin over the course of the study and exhibit thereafter an increased disease severity score and augmented back skin as well as ear thickness. Pro-inflammatory cytokines TNFα, IL-17A and IL-23 are increased in psoriatic back skin from IMQ-treated mice compared to healthy control skin.

Effect of psoriasis severity in IMQ-treated mice following treatment with 5, 10 or

14 mg/kg/day of PDE4 inhibitor Apremilast alone, fixed dose of SCFA alone, and combination of 5, 10 or 14 mg/kg/day of PDE4 inhibitor Apremilast with fixed dose of SCFA are evaluated for anti-inflammatory efficacy.

Further, combination treatment with ascending doses of Apremilast plus fixed dose SCFA are assessed for additive/synergistic effects compared to monotherapy with SCFA or to equal dose of monotherapy with Apremilast.

### Skin Thickening

Effect of psoriasis severity in IMQ-treated mice following treatment with 5, 10 or

14 mg/kg/day of PDE4 inhibitor Apremilast alone, fixed dose of SCFA alone, and combination of 5, 10, or 14 mg/kg/day of PDE4 inhibitor Apremilast with fixed dose of SCFA are evaluated for skin thickening of psoriatic back skin and ear skin.

Further, combination treatment with ascending doses of Apremilast plus fixed dose SCFA are assessed for additive/synergistic effects compared to monotherapy with SCFA or to equal dose of monotherapy with Apremilast for inhibition of ear thickness and decrease in back skin thickness.

### Body Weight

Body weight was determined daily in all mice over the whole period of each study as described in Example 4.

### Cytokine Analysis—Multiplex Assay

Human pro-inflammatory cytokines (e.g., TNFα, IL-17A and IL-23) of each mouse were measured by multiplex ELISA in back skin biopsies collected on day 6 after IMQ psoriasis study.

With regards to observations of animal well-being, SCFA as well as all tested doses of Apremilast or combination treatments are well tolerated during the study duration.

### Example 9: Combination Treatment of Skin Disorders Using Short Chain Fatty Acids

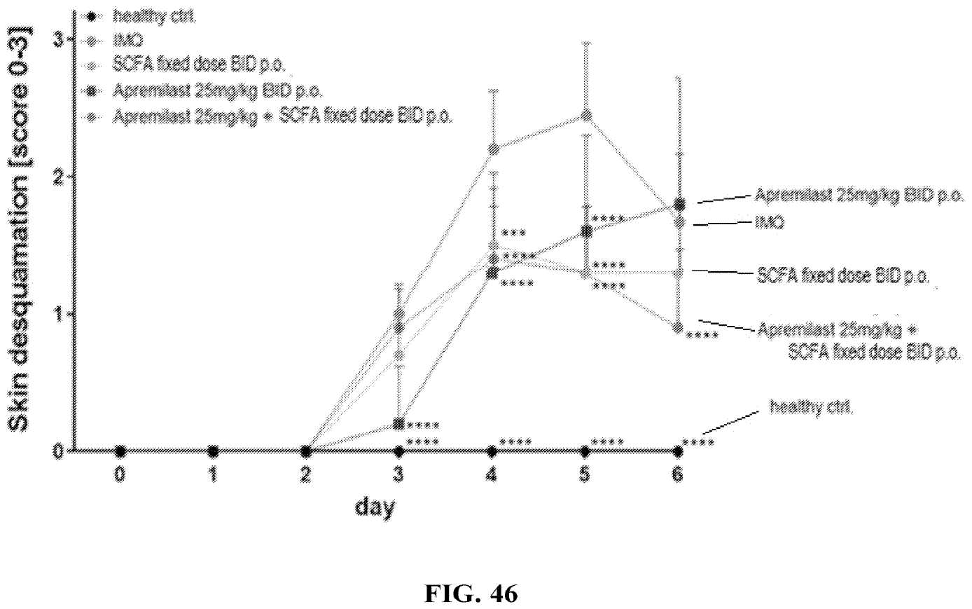

Various assays were designed and tested in the imiquimod (IMQ)-induced psoriasis model in mice as shown and described for FIGS. 46-49. The anti-inflammatory activity of PDE4-inhibitor Apremilast and SCFAs were evaluated in combination vs. monotherapy in the mouse imiquimod (IMQ)-induced psoriasis model.

### Imiquimod (IMQ)-Induced Psoriasis Model

A mouse IMQ-induced psoriasis (IMQ Ps) model was prepared as described in Example 4. IMQ psoriasis was characterized by rapid induction of skin inflammation with pathologic and histologic resemblance to human psoriasis, including skin erythema, skin scaling and epidermal thickening (acanthosis), accompanied with immune cell infiltration.

Before starting treatment on the day of disease induction (day 0), mice were randomized based on body weight to the respective group. To evaluate the anti-inflammatory efficacy of the two compounds in mouse IMQ psoriasis, mice were treated twice daily (BID) by oral gavage with 25 mg/kg of Apremilast (in 0.5% CMC/0.025% Tween80 in sterile water; m/m/v) alone, with fixed dose of SCFA (in ddH2O) alone, or with 25 mg/kg Apremilast in combination with fixed dose of SCFA. Healthy and IMQ disease controls were orally treated in parallel with vehicle only. All treatments started with first application of IMQ on day 0.

In these experiments, the fixed dose of SCFA comprised 120 mg/kg/day of Ca-propionate and 1,100 mg/kg/day mixture of salts (Ca-butyrate and Mg-butyrate).

### Skin Scaling

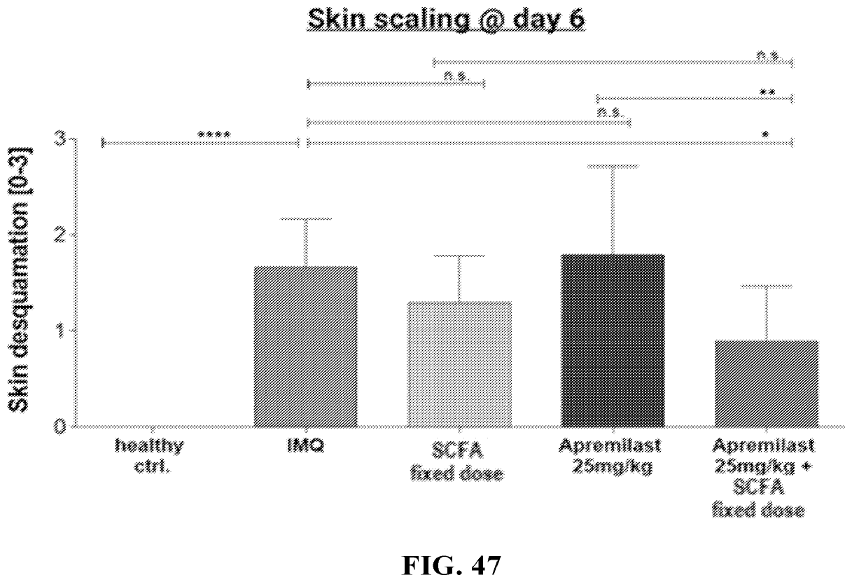

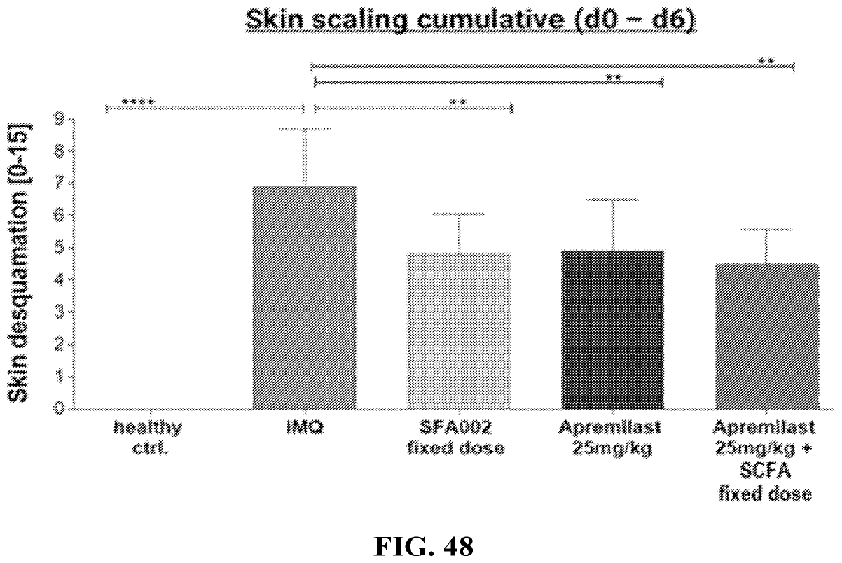

In separate evaluation of skin scaling, one of the disease severity score parameters, revealed that combination treatment significantly (p<0.001) reduced the desquamation of the back skin starting on day 3 until the end of the study (FIG. 46). Monotherapy of SFA002 and Apremilast was also able to inhibit the skin scaling from day 3 to day 5. Cumulative evaluation of skin scaling over the entire course of the study showed significant and similar beneficial effects of all treatments with a p value of p<0.01 for Apremilast, SFA002 and combination therapy (Apremilast plus SFA002) (FIG. 48). However, at the end of the study (d6), only the combination approach Apremilast plus SFA002 continued to show significant reduction of skin scaling (−50%) compared to all IMQ-induced comparator groups including Apremilast alone (FIG. 47).

### Body Weight

Body weight was determined daily in all mice over the whole period of each study as described in Example 4.

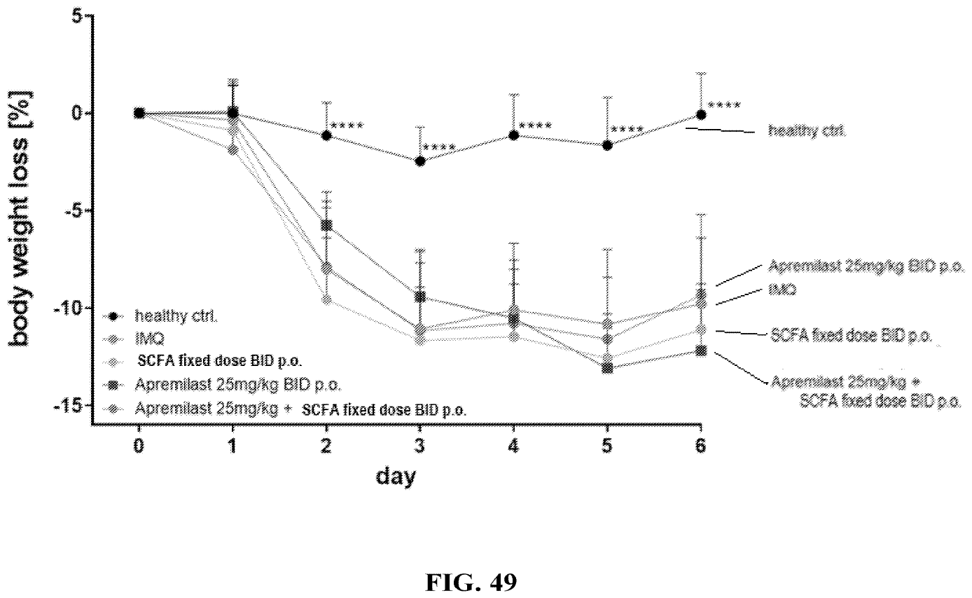

As known, IMQ induces a slight body weight loss within the first two to three days (FIG. 49). Both, Apremilast and SCFA whether in combination or alone showed no positive influence on body weight loss by treatment compared to vehicle-treated IMQ disease controls (FIG. 49).

With regards to observations of animal well-being, SCFA and all tested doses of Apremilast or combination treatments were well tolerated during the study duration.

### Example 10: Combination Treatment of Skin Disorders Using Short Chain Fatty Acids

Various assays are designed and tested in the imiquimod (IMQ)-induced psoriasis model in mice. The anti-inflammatory activity of PDE4-inhibitor Apremilast and SCFAs are evaluated in combination vs. monotherapy in the mouse imiquimod (IMQ)-induced psoriasis model.

### Imiquimod (IMQ)-Induced Psoriasis Model

A mouse IMQ-induced psoriasis (IMQ Ps) model is prepared as described in Example 4.

Before starting treatment on the day of disease induction (day 0), mice are randomized based on body weight to the respective group. To evaluate the anti-inflammatory efficacy of the two compounds in mouse IMQ psoriasis, mice are treated twice daily (BID) by oral gavage with 25 mg/kg of Apremilast (in 0.5% CMC/0.025% Tween80 in sterile water; m/m/v) alone, with fixed dose of SCFA (in ddH2O) alone, or with 25 mg/kg Apremilast in combination with fixed dose of SCFA. Healthy and IMQ disease controls are orally treated in parallel with vehicle only. All treatments start with first application of IMQ on day 0.

In these experiments, the SCFAs administered were 120 mg/kg/day of Ca-propionate and 1,100 mg/kg/day mixture of salts (Ca-butyrate and Mg-butyrate).

### Psoriasis Severity Score

Disease Severity Score is evaluated on a daily base as described in Example 4.

During the study, the disease severity score (comprising erythema, skin scaling and thickening) resembling the psoriasis severity is assessed daily at the psoriatic back skin. Next to this, the extent of ear thickening of the IMQ-treated right ear is also evaluated daily.

All IMQ control mice develop a psoriasis phenotype at the back skin over the course of the study and exhibit thereafter an increased disease severity score and augmented back skin as well as ear thickness. Pro-inflammatory cytokines TNFα, IL-17A and IL-23 are increased in psoriatic back skin from IMQ-treated mice compared to healthy control skin.

Effect of psoriasis severity in IMQ-treated mice following treatment with 50 mg/kg/day of PDE4 inhibitor Apremilast alone, fixed dose of SCFA alone, and combination of 50 mg/kg/day of PDE4 inhibitor Apremilast with fixed dose of SCFA are evaluated for anti-inflammatory efficacy.

Further, combination treatment with ascending doses of Apremilast plus fixed dose SCFA are assessed for additive/synergistic effects compared to monotherapy with SCFA or to equal dose of monotherapy with Apremilast.

### Skin Thickening

Effect of psoriasis severity in IMQ-treated mice following treatment with 50 mg/kg/day of PDE4 inhibitor Apremilast alone, fixed dose of SCFA alone, and combination of 50 mg/kg/day of PDE4 inhibitor Apremilast with fixed dose of SCFA are evaluated for skin thickening of psoriatic back skin and ear skin.

Further, combination treatment with ascending doses of Apremilast plus fixed dose SCFA are assessed for additive/synergistic effects compared to monotherapy with SCFA or to equal dose of monotherapy with Apremilast for inhibition of ear thickness and decrease in back skin thickness.

### Body Weight

Body weight was determined daily in all mice over the whole period of each study as described in Example 4.

### Cytokine Analysis—Multiplex Assay

Human pro-inflammatory cytokines (e.g., TNFα, IL-17A and IL-23) of each mouse are measured by multiplex ELISA in back skin biopsies collected on day 6 after IMQ psoriasis study.

With regards to observations of animal well-being, SCFA and all tested doses of Apremilast or combination treatments are well tolerated during the study duration.

### Example 11: Treatment of Skin Disorders Using Short Chain Fatty Acids

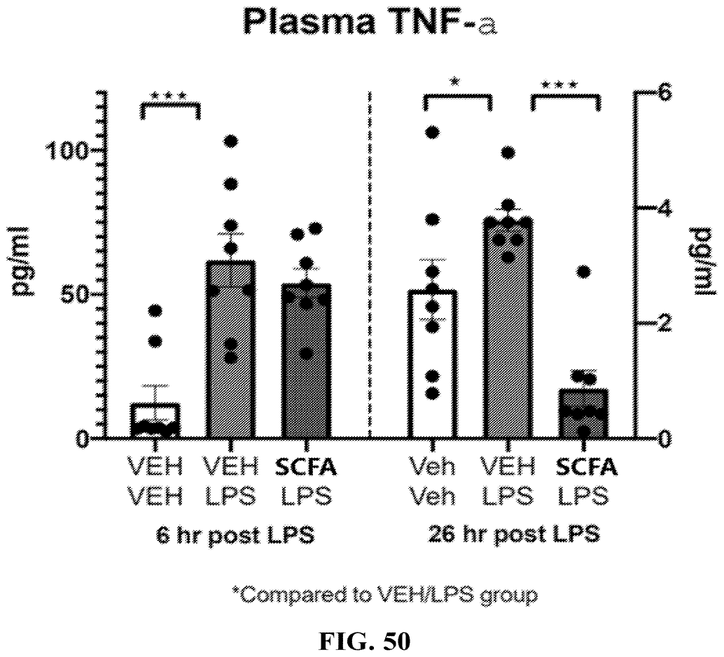

Various assays were designed and tested in an LPS animal model of inflammation as shown and described for FIG. 50. The plasma TNF-α levels were evaluated at 6 hours and 26 hours post-LPS. Administration of SCFA showed a significant and rapid downregulation of TNF-α within 26 hours following treatment (FIG. 50).

### Example 12: Combination Treatment of Skin Disorders Using Short Chain Fatty Acids

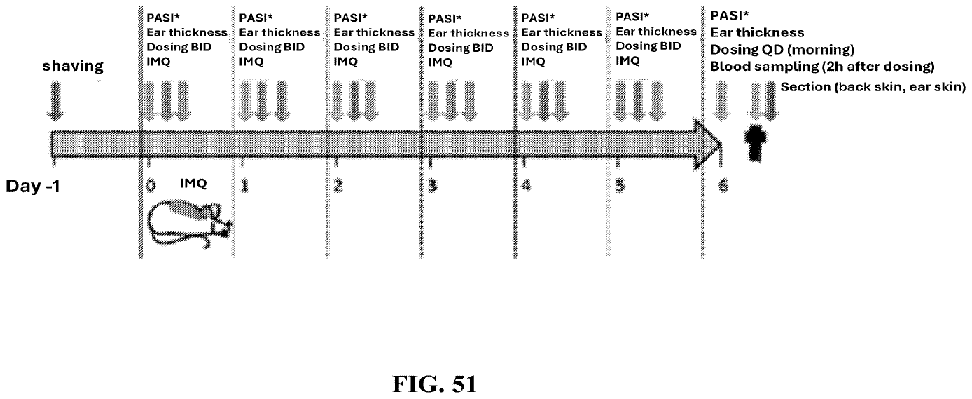

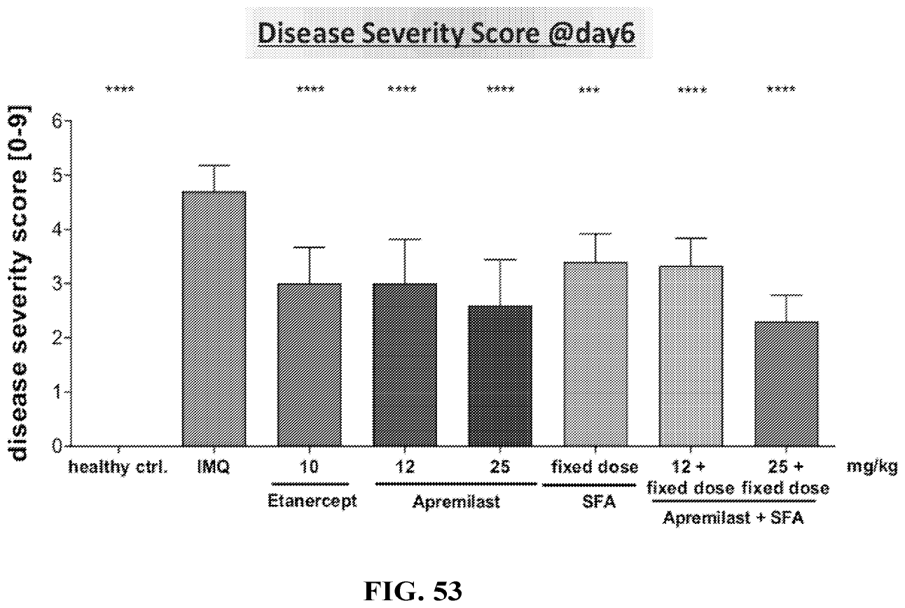

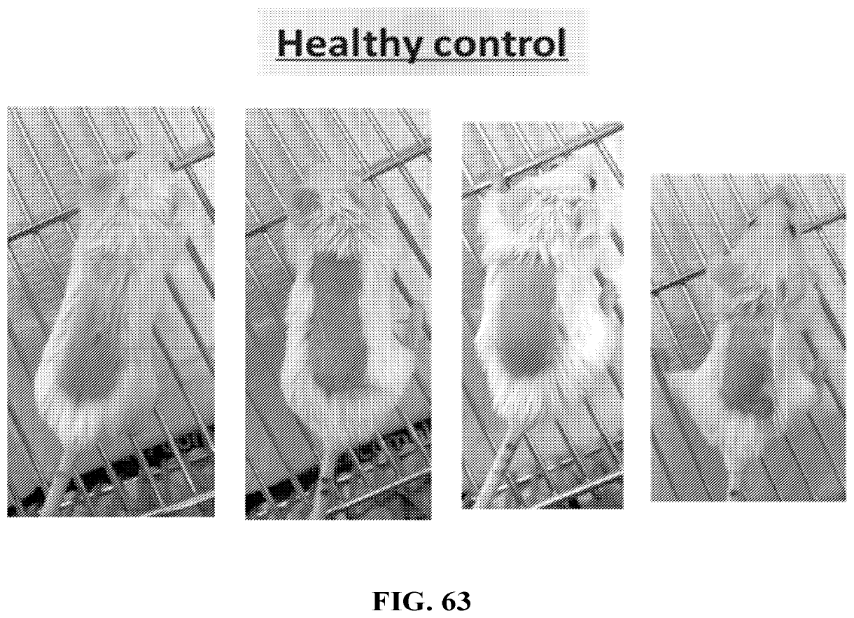

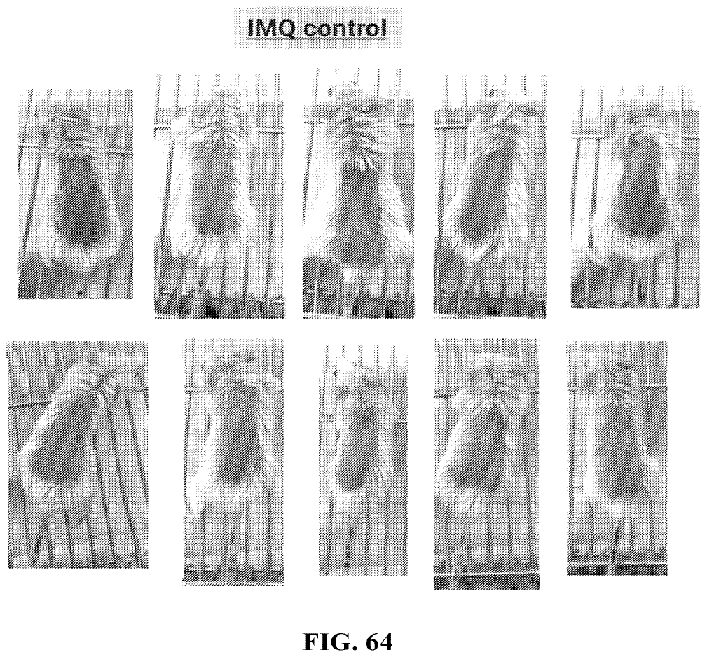

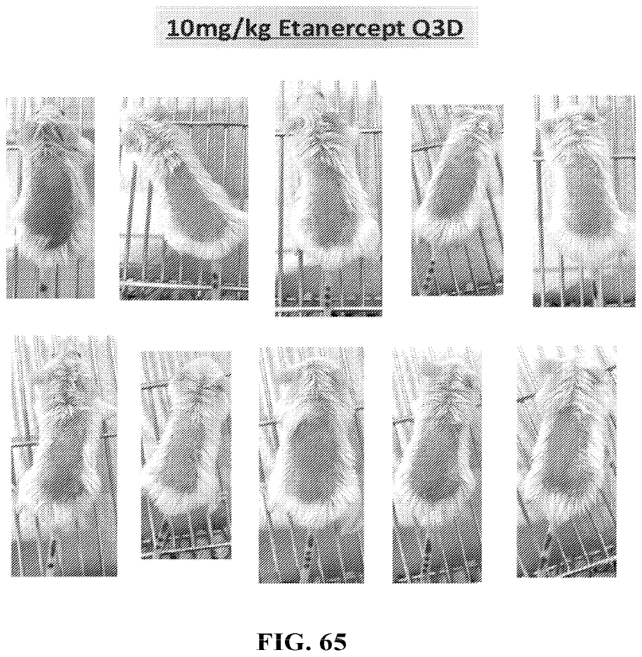

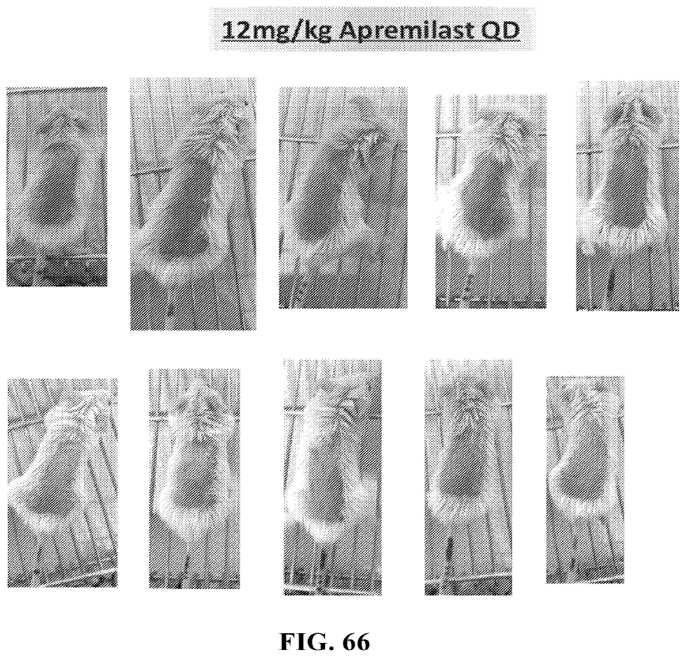

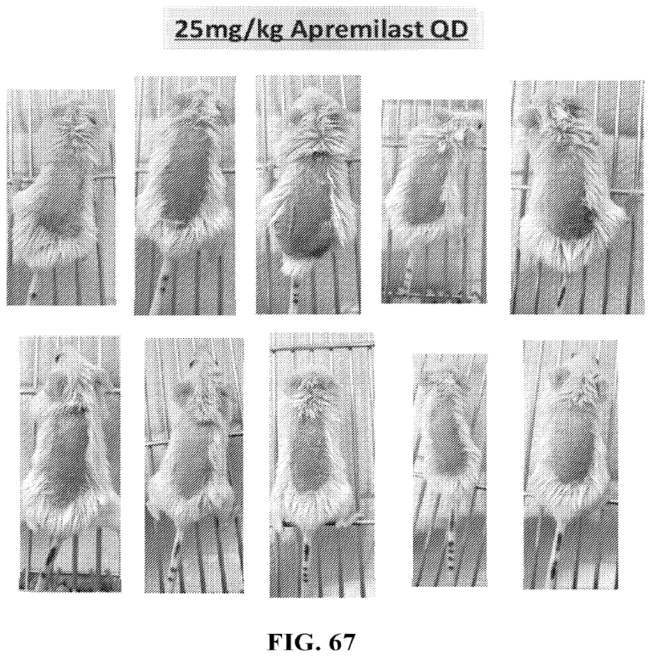

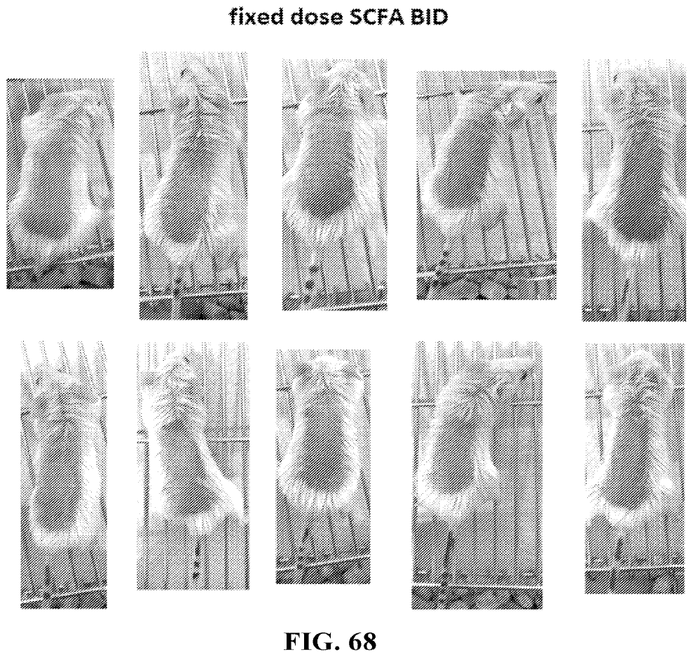

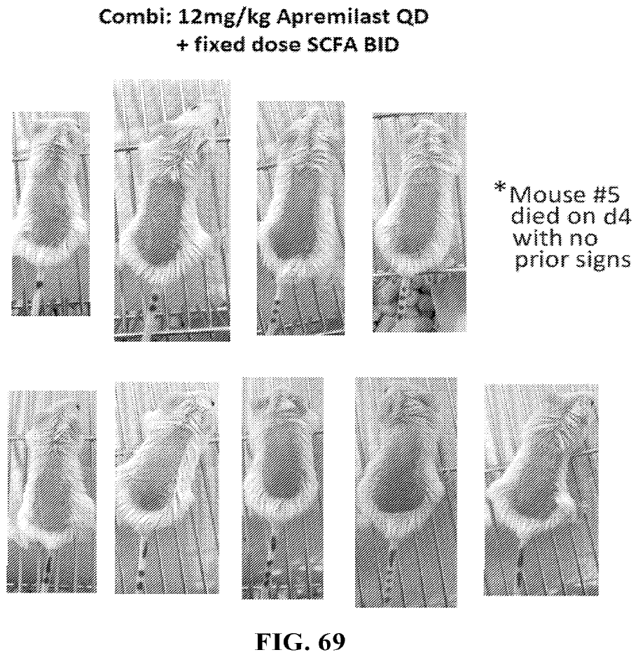

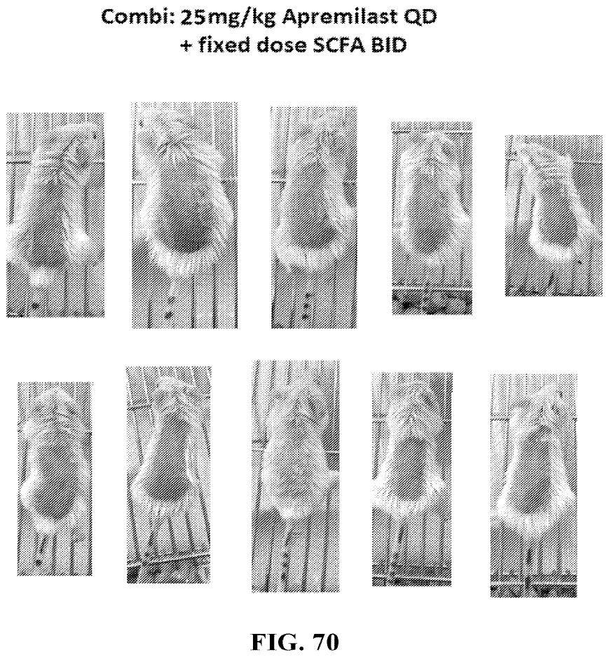

Various assays were designed and tested in an imiquimod (IMQ)-induced psoriasis model in mice as shown and described for FIGS. 51-70. The anti-inflammatory activity of PDE4-inhibitor Apremilast and SCFAs were evaluated in combination vs. monotherapy in the mouse imiquimod (IMQ)-induced psoriasis model.

### Imiquimod (IMQ)-Induced Psoriasis Model

A mouse IMQ-induced psoriasis (IMQ Ps) model was prepared using female Balb/c mice (8 weeks of age at ordering, and approximately 19 g-20 g, Charles River, Sulzfeld, Germany; n=10 mice/group, exception healthy controls n=4), as described in Example 4 (FIG. 51).

Before starting treatment on the day of disease induction (day 0), mice are randomized based on body weight to the respective group. To evaluate the anti-inflammatory efficacy of the two compounds in mouse IMQ psoriasis, mice are treated twice daily (BID) by oral gavage with one of the following: (1) 12 mg/kg of Apremilast (in 0.5% CMC/0.025% Tween80 in sterile water; m/m/v) alone, (2) 25 mg/kg of Apremilast (in 0.5% CMC/0.025% Tween80 in sterile water; m/m/v) alone, (3) fixed dose of SCFA (in ddH2O) alone, (4) 12 mg/kg Apremilast in combination with fixed dose of SCFA, (5) 25 mg/kg Apremilast in combination with fixed dose of SCFA. Healthy and IMQ disease controls are orally treated in parallel with vehicle only. Etanercept (10 mg/kg Q3D s.c.) served as a technical control. All treatments start with a first application of IMQ on day 0.

In these experiments, the SCFAs administered were 60 mg/kg/day of Ca-propionate and 1,100 mg/kg/day mixture of salts (Ca-butyrate and Mg-butyrate).

IMQ psoriasis is characterized by rapid induction of skin inflammation with pathologic and histologic resemblance to human psoriasis, including skin erythema, skin scaling and epidermal thickening (acanthosis), accompanied with immune cell infiltration.

### Psoriasis Severity Score

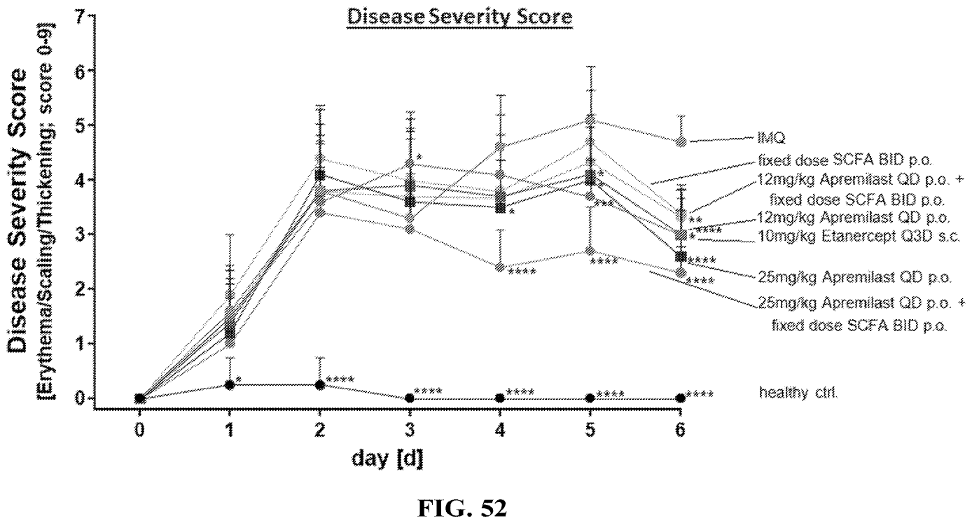

Disease Severity Score was evaluated in IMQ-treated mice on a daily base as described in Example 4. During the study, parameters including the disease severity score (erythema, scaling, thickening) were assessed at psoriatic back skin (FIGS. 52-56).

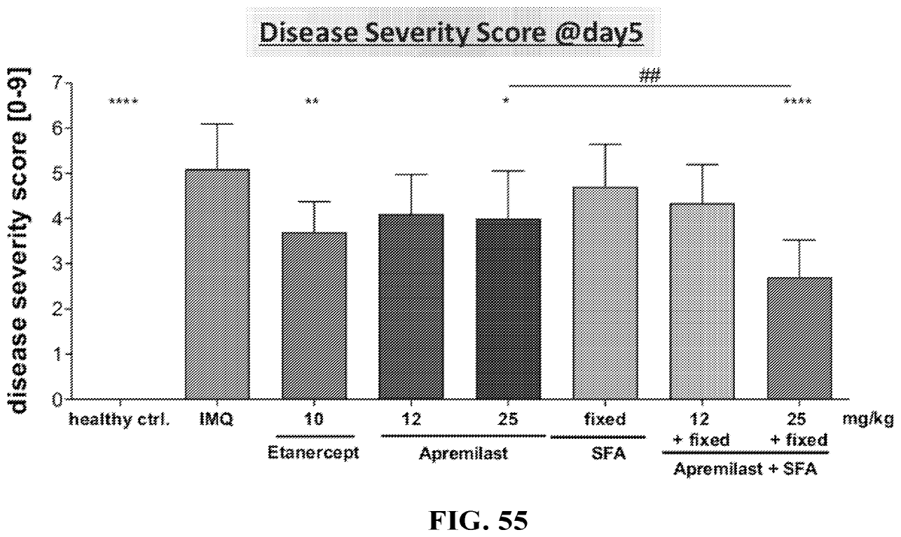

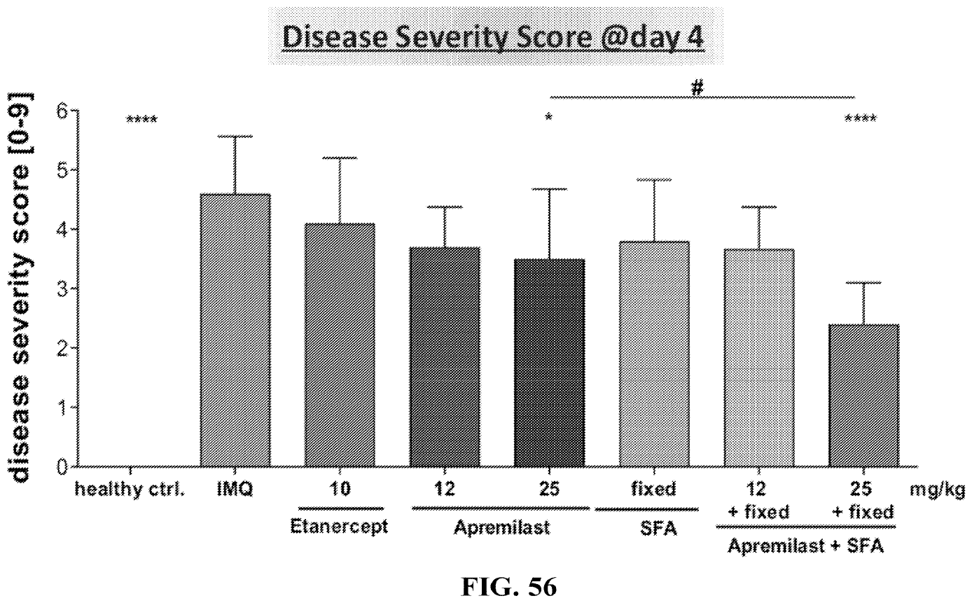

Reduction of psoriasis severity score following treatment with a monotherapy of 12 mg/kg of Apremilast did not reach significance at day 4 and day 5 (FIG. 55 and FIG. 56). However, treatment with monotherapy of 25 mg/kg of Apremilast reduced the psoriasis severity score in IMQ-treated mice at day 4 of the study (p<0.05), and these effects were maintained at the end of the study at day 6 (FIGS. 52-53 and FIGS. 55-56).

Treatment with combination treatment with fixed dose of SCFA and 25 mg/kg of Apremilast significantly reduced the disease severity score throughout the study, including at day 4 (p<0.0001) and day 5 (p<0.0001) (FIGS. 52-53 and FIGS. 55-56). At day 4 and day 5, the combination therapy of fixed dose of SCFA and 25 mg/kg of Apremilast exhibited an additive or synergistic effect with respect to reduction of disease severity score compared to the monotherapy of 25 mg/kg of Apremilast (FIG. 55 and FIG. 56).

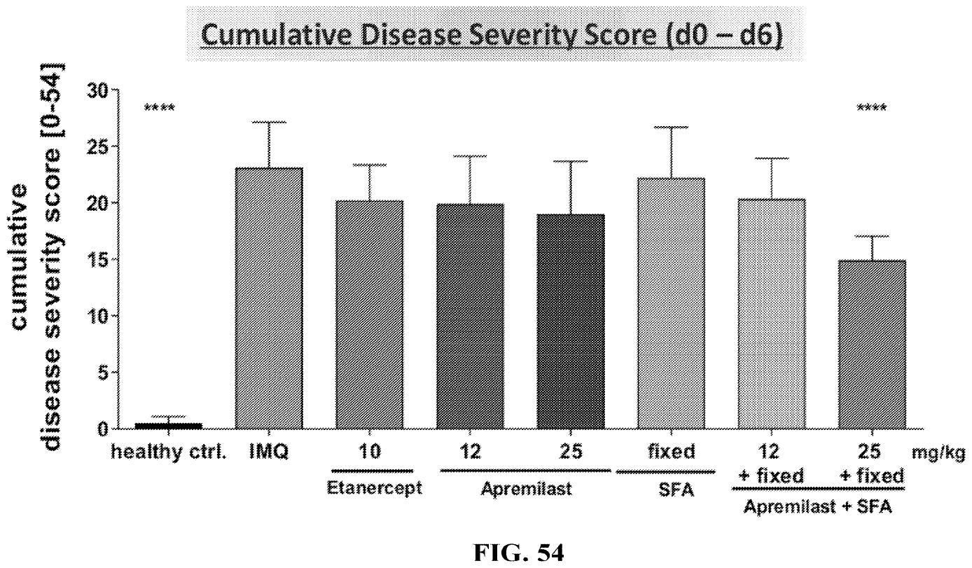

Cumulative evaluation of disease severity score over the entire course of the study showed significant beneficial effects with a p value of p<0.001 for a combination therapy of fixed dose SCFA and 25 mg/kg of Apremilast (FIG. 54). Among all the IMQ-induced comparator groups tested, only the combination treatment of fixed dose of SCFA and 25 mg/kg of Apremilast showed a significant reduction of cumulative disease severity score (FIG. 54).

### Skin Scaling

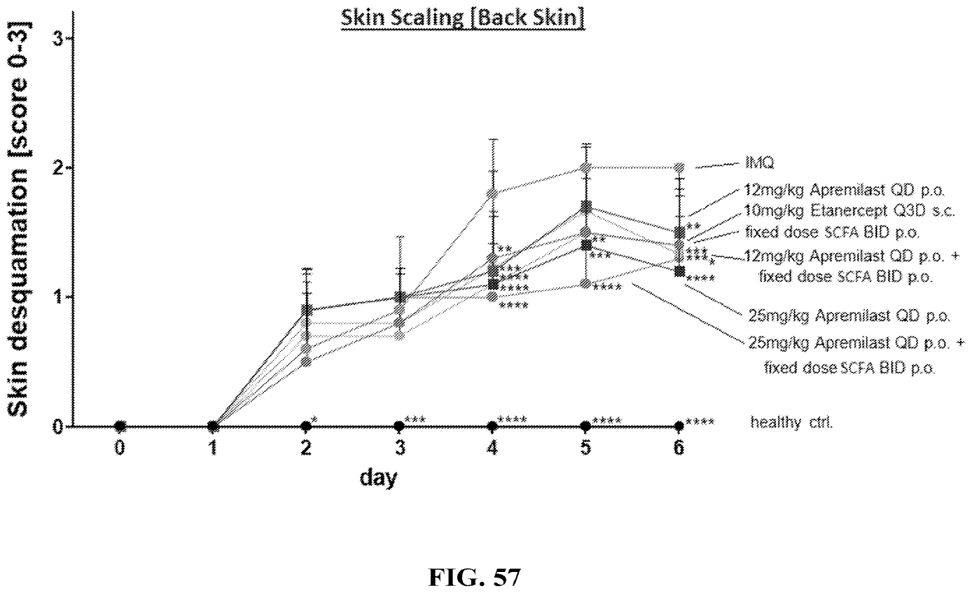

Skin scaling, one of the disease severity score parameters, was evaluated in IMQ-treated mice (FIGS. 57-59).

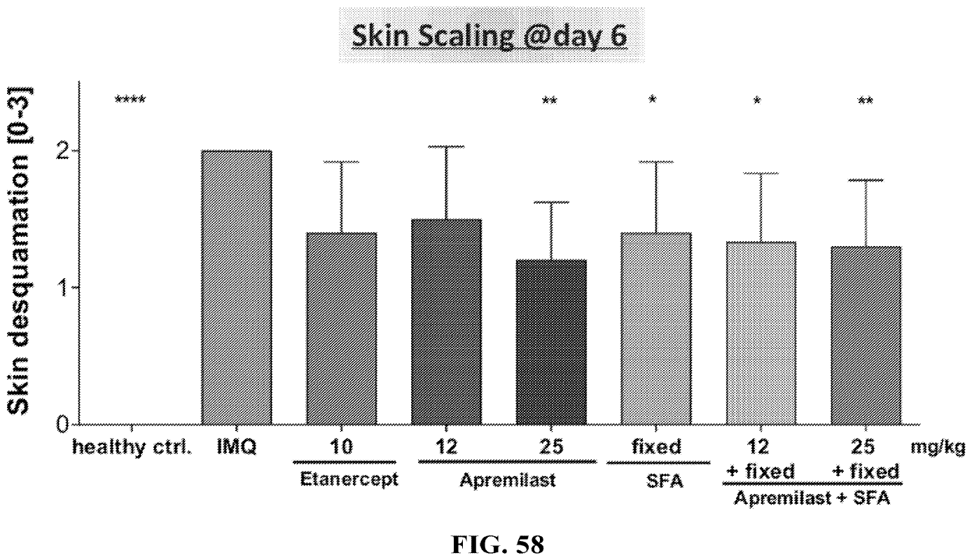

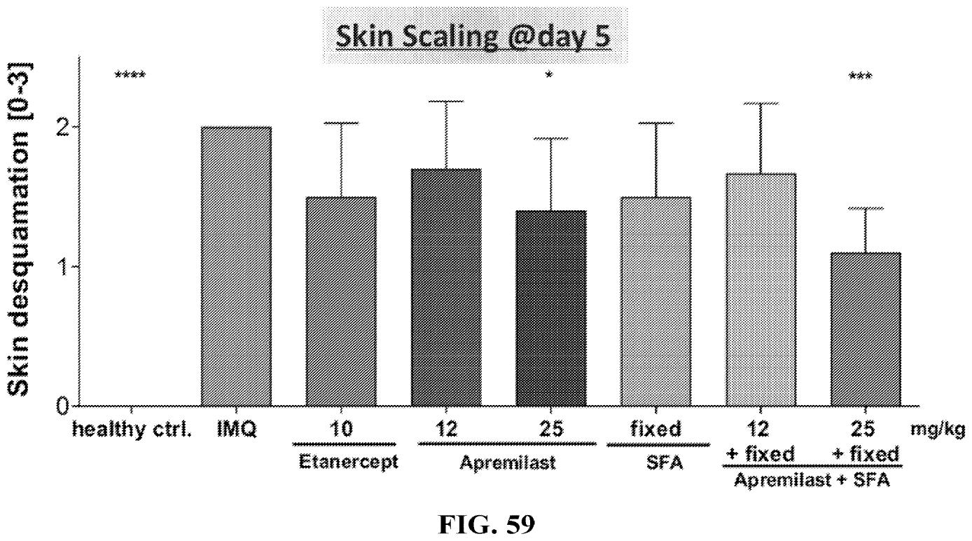

Treatment with a monotherapy of 25 mg/kg of Apremilast significantly inhibited skin scaling on day 5 (p<0.05) and day 6 (p<0.01) (FIG. 58 and FIG. 59). Treatment with the combination treatment of fixed dose of SCFA and 25 mg/kg of Apremilast significantly inhibited skin scaling on day 5 (p<0.005) and day 6 (p<0.01) (FIG. 58 and FIG. 59).

At day 5 of the study, treatment with 25 mg/kg of Apremilast alone significantly (p<0.05) reduced the desquamation of the back skin, and the combination treatment of fixed dose of SCFA and 25 mg/kg of Apremilast further reduced desquamation of the back skin (p<0.005) (FIG. 57 and FIG. 59). At day 5, the combination therapy of fixed dose of SCFA and 25 mg/kg of Apremilast exhibited an additive or synergistic effect with respect to reduction of skin scaling compared to the monotherapy of 25 mg/kg of Apremilast (FIG. 59).

Treatment with a monotherapy of fixed dose of SCFA or a combination therapy of fixed dose of SCFA and 12 mg/kg of Apremilast significantly inhibited skin scaling by day 6 (p<0.05) (FIGS. 57-59). Reduction of skin scaling following treatment with a monotherapy of 12 mg/kg of Apremilast did not reach significance at day 6 (FIG. 58).

### Ear Thickening

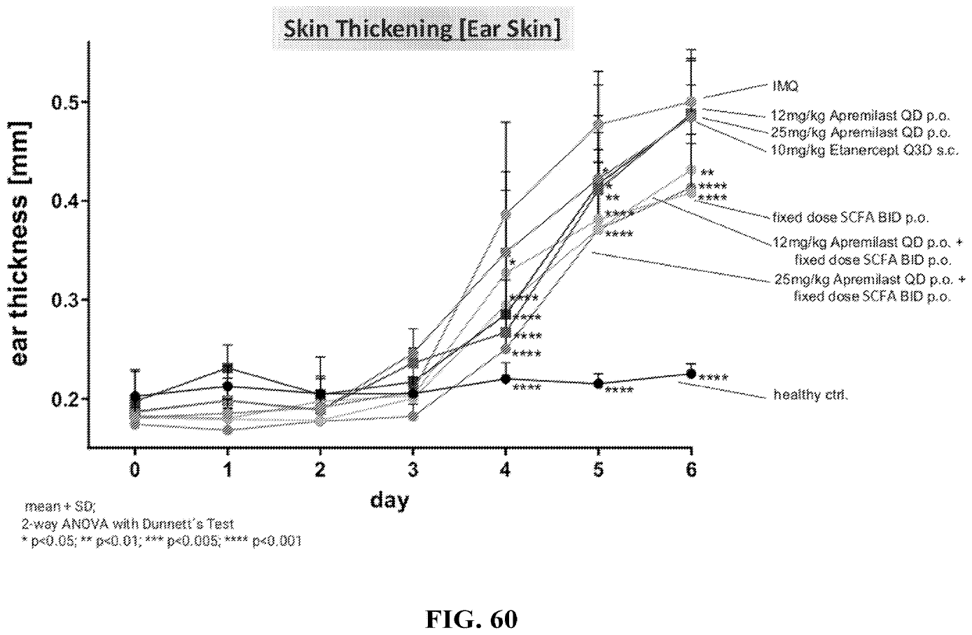

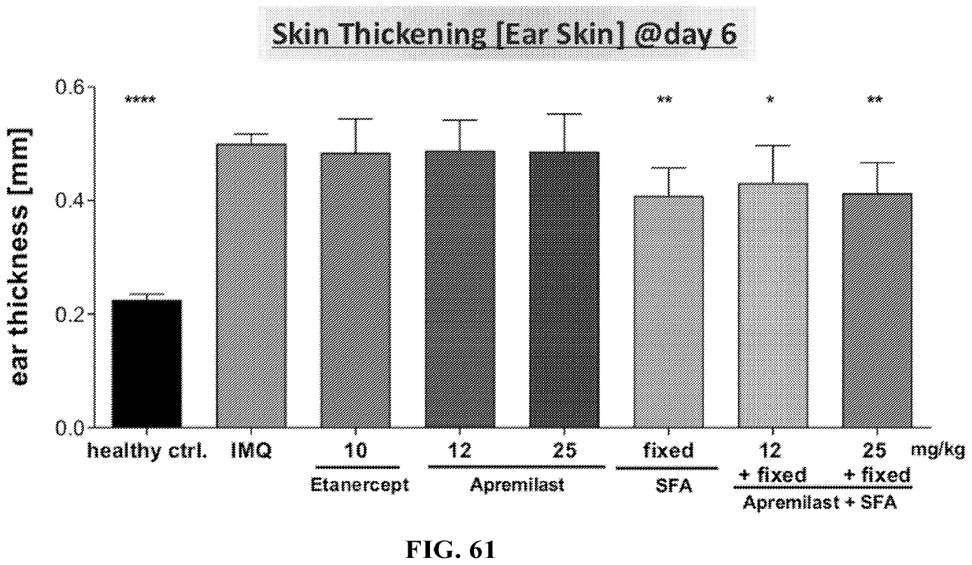

Ear thickening, one of the disease severity score parameters, was evaluated in IMQ-treated mice (FIG. 60 and FIG. 61). The extent of ear thickness of the IMQ-treated right ear was determined daily by using an automatized caliper (Bayer AG, Germany) as described in Example 4.

Treatment with fixed dose SCFA alone and combination therapy of fixed dose of SCFA and 25 mg/kg of Apremilast significantly inhibited the skin thickening of ear skin (p<0.01) at day 6 (FIG. 61). Ear thickness was also inhibited by treatment with a combination therapy of fixed dose of SCFA and 12 mg/kg of Apremilast (p<0.05) at day 6 (FIG. 61). However, treatment with both monotherapy doses of Apremilast (12 mg/kg and 25 mg/kg) did not show any inhibitory influence on ear thickness at day 6 (FIG. 61).

Combination therapies of fixed dose of SCFA and Apremilast exhibited an additive or synergistic effect with respect to reduction of ear skin thickening compared to monotherapy of corresponding doses of Apremilast (FIG. 60 and FIG. 61).

### Body Weight

Body weight was determined daily in all mice over the whole period of each study as described in Example 4.

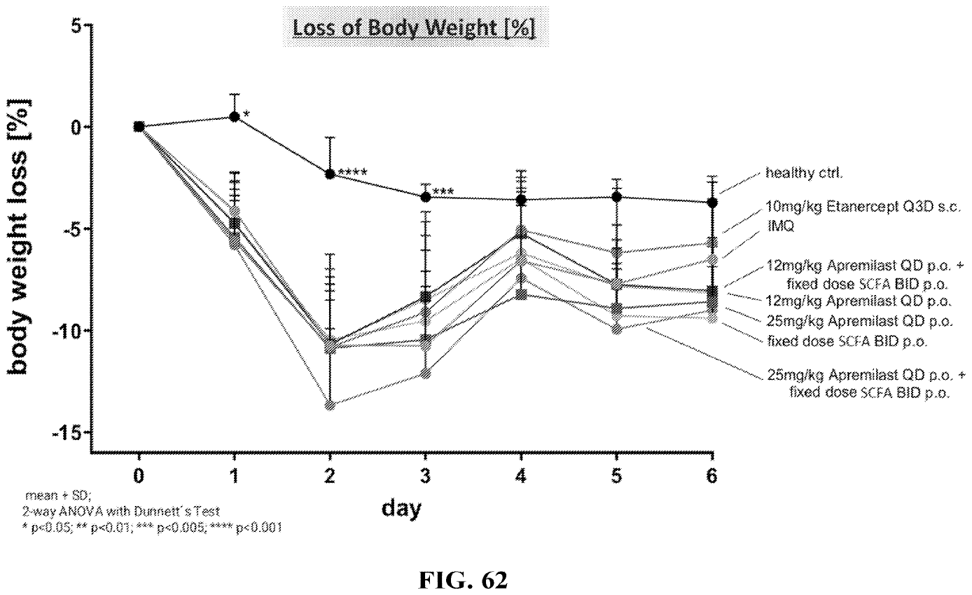

As known, IMQ induces a slight body weight loss within the first two to three days (FIG. 62). Both, Apremilast and SCFA whether in combination or alone showed no positive influence on body weight loss by treatment compared to vehicle-treated IMQ disease controls (FIG. 62).

With regards to observations of animal well-being, SCFA and all tested doses of Apremilast or combination treatments were well tolerated during the study duration.

### EMBODIMENTS

The following non-limiting embodiments provide illustrative examples of the disclosure, but do not limit the scope of the disclosure.

Embodiment 1. A method of treating a condition, the method comprising: a) administering a first pharmaceutical composition to a subject in need thereof, wherein the first pharmaceutical composition comprises a therapeutically-effective amount of a compound that is a short chain fatty acid or a pharmaceutically-acceptable salt thereof; and b) administering a second pharmaceutical composition to the subject, wherein the second pharmaceutical composition comprises a therapeutically-effective amount of a phosphodiesterase-4 (PDE4) inhibitor.

Embodiment 2. The method of embodiment 1, wherein the condition is a skin disorder.

Embodiment 3. The method of embodiment 1 or 2, wherein the condition is psoriasis.

Embodiment 4. The method of any one of embodiments 1-3, wherein the condition is an autoimmune disorder.

Embodiment 5. The method of any one of embodiments 1-4, wherein the short chain fatty acid is butyric acid.

Embodiment 6. The method of any one of embodiments 1-5, wherein the short chain fatty acid is propionic acid.

Embodiment 7. The method of any one of embodiments 1-6, wherein the compound is butyric acid or a pharmaceutically-acceptable salt thereof, and wherein the first pharmaceutical composition further comprises an additional short chain fatty acid or a pharmaceutically-acceptable salt thereof, wherein the additional short chain fatty acid is propionic acid or a pharmaceutically-acceptable salt thereof.

Embodiment 8. The method of any one of embodiments 1-7, wherein the PDE4 inhibitor is Apremilast.

Embodiment 9. The method of any one of embodiments 1-8, wherein the therapeutically-effective amount of the PDE4 inhibitor is from about 10 mg to about 15 mg.

Embodiment 10. The method of any one of embodiments 1-9, wherein the first pharmaceutical composition further comprises vitamin D3 in an amount of from about 50 IU to about 200 IU.

Embodiment 11. The method of any one of embodiments 1-10, wherein the first pharmaceutical composition is formulated for oral administration.

Embodiment 12. A method of treating a condition, the method comprising administering to a subject in need thereof a therapeutically-effective amount of a first pharmaceutical composition comprising at least one SCFA and a reduced amount of a second therapy, wherein the reduced amount of the second therapy is therapeutically effective for treating the condition in combination with the therapeutically-effective amount of the first pharmaceutical composition comprising at least one SCFA, and wherein the reduced amount of the second therapy is less than an amount of the second therapy that is therapeutically effective for the condition in absence of the therapeutically-effective amount of the first pharmaceutical composition comprising at least one SCFA.

Embodiment 13. The method of embodiment 12, wherein the condition is a skin disorder.

Embodiment 14. The method of embodiment 12 or 13, wherein the condition is psoriasis.

Embodiment 15. The method of any one of embodiments 12-14, wherein the condition is an autoimmune disorder.

Embodiment 16. The method of any one of embodiments 12-15, wherein the first pharmaceutical composition comprises butyric acid or a pharmaceutically-acceptable salt thereof.

Embodiment 17. The method of any one of embodiments 12-16, wherein the first pharmaceutical composition comprises propionic acid or a pharmaceutically-acceptable salt thereof

Embodiment 18. The method of any one of embodiments 12-17, wherein the first pharmaceutical composition comprises at least two SCFAs.

Embodiment 19. The method of any one of embodiments 12-18, wherein the first pharmaceutical composition comprises butyric acid or a pharmaceutically-acceptable salt thereof and propionic acid or a pharmaceutically-acceptable salt thereof.

Embodiment 20. The method of any one of embodiments 12-19, wherein the first pharmaceutical composition comprises an inorganic magnesium salt.

Embodiment 21. The method of any one of embodiments 12-20, wherein the first pharmaceutical composition comprises Vitamin D3.

Embodiment 22. The method of any one of embodiments 12-21, wherein the second therapy comprises a PDE4 inhibitor.

Embodiment 23. The method of embodiment 22, wherein the PDE4 inhibitor is Apremilast.

Embodiment 24. The method of any one of embodiments 12-23, wherein the second therapy exhibits synergy with the first pharmaceutical composition.

Embodiment 25. The method of any one of embodiments 12-24, wherein the reduced amount of the second therapy comprises administering a reduced dosage of the second therapy to the subject.

Embodiment 26. The method of any one of embodiments 12-25, wherein the reduced amount of the second therapy comprises administering the second therapy to the subject at a reduced frequency.

Embodiment 27. The method of any one of embodiments 12-26, wherein the reduced amount of the second therapy comprises administering a reduced dosage of the second therapy to the subject at a reduced frequency.

Embodiment 28. The method of any one of embodiments 12-27, wherein the treating comprises causing reduction of a psoriatic lesion in the subject.

Embodiment 29. The method of any one of embodiments 12-28, wherein the treating comprises causing disappearance of a psoriatic lesion in the subject.

What is claimed is:

1. A method of treating a condition, the method comprising:
a. administering a first pharmaceutical composition to a subject in need thereof, wherein the first pharmaceutical composition comprises a therapeutically-effective amount of a compound that is a short chain fatty acid or a pharmaceutically-acceptable salt thereof; and
b. administering a second pharmaceutical composition to the subject, wherein the second pharmaceutical composition comprises a therapeutically-effective amount of a phosphodiesterase-4 (PDE4) inhibitor.

2. The method of claim 1, wherein the condition is a skin disorder.

3. The method of claim 1 or 2, wherein the condition is psoriasis.

4. The method of any one of claims 1-3, wherein the condition is an autoimmune disorder.

5. The method of any one of claims 1-4, wherein the short chain fatty acid is butyric acid.

6. The method of any one of claims 1-5, wherein the short chain fatty acid is propionic acid.

7. The method of any one of claims 1-6, wherein the compound is butyric acid or a pharmaceutically-acceptable salt thereof, and wherein the first pharmaceutical composition further comprises an additional short chain fatty acid or a pharmaceutically-acceptable salt thereof, wherein the additional short chain fatty acid is propionic acid or a pharmaceutically-acceptable salt thereof.

8. The method of any one of claims 1-7, wherein the PDE4 inhibitor is Apremilast.

9. The method of any one of claims 1-8, wherein the therapeutically-effective amount of the PDE4 inhibitor is from about 10 mg to about 15 mg.

10. The method of any one of claims 1-9, wherein the first pharmaceutical composition further comprises vitamin D3 in an amount of from about 50 IU to about 200 IU.

11. The method of any one of claims 1-10, wherein the first pharmaceutical composition is formulated for oral administration.

12. A method of treating a condition, the method comprising administering to a subject in need thereof a therapeutically-effective amount of a first pharmaceutical composition comprising at least one SCFA and a reduced amount of a second therapy, wherein the reduced amount of the second therapy is therapeutically effective for treating the condition in combination with the therapeutically-effective amount of the first pharmaceutical composition comprising at least one SCFA, and wherein the reduced amount of the second therapy is less than an amount of the second therapy that is therapeutically effective for the condition in absence of the therapeutically-effective amount of the first pharmaceutical composition comprising at least one SCFA.

13. The method of claim 12, wherein the condition is a skin disorder.

14. The method of claim 12 or 13, wherein the condition is psoriasis.

15. The method of any one of claims 12-14, wherein the condition is an autoimmune disorder.

16. The method of any one of claims 12-15, wherein the first pharmaceutical composition comprises butyric acid or a pharmaceutically-acceptable salt thereof.

17. The method of any one of claims 12-16, wherein the first pharmaceutical composition comprises propionic acid or a pharmaceutically-acceptable salt thereof.

18. The method of any one of claims 12-17, wherein the first pharmaceutical composition comprises at least two SCFAs.

19. The method of any one of claims 12-18, wherein the first pharmaceutical composition comprises butyric acid or a pharmaceutically-acceptable salt thereof and propionic acid or a pharmaceutically-acceptable salt thereof.

20. The method of any one of claims 12-19, wherein the first pharmaceutical composition comprises an inorganic magnesium salt.

21. The method of any one of claims 12-20, wherein the first pharmaceutical composition comprises Vitamin D3.

22. The method of any one of claims 12-21, wherein the second therapy comprises a PDE4 inhibitor.

23. The method of claim 22, wherein the PDE4 inhibitor is Apremilast.

24. The method of any one of claims 12-23, wherein the second therapy exhibits synergy with the first pharmaceutical composition.

25. The method of any one of claims 12-24, wherein the reduced amount of the second therapy comprises administering a reduced dosage of the second therapy to the subject.

26. The method of any one of claims 12-25, wherein the reduced amount of the second therapy comprises administering the second therapy to the subject at a reduced frequency.

27. The method of any one of claims 12-26, wherein the reduced amount of the second therapy comprises administering a reduced dosage of the second therapy to the subject at a reduced frequency.

28. The method of any one of claims 12-27, wherein the treating comprises causing reduction of a psoriatic lesion in the subject.

29. The method of any one of claims 12-28, wherein the treating comprises causing disappearance of a psoriatic lesion in the subject.
